(19) United States
(12) Patent Application Publication
(10) Pub. No.: US 2024/0392379 A1
(43) Pub. Date: Nov. 28, 2024

(54) METHODS OF TREATING CANCERS HAVING A BIALLELIC LOSS OF FUNCTION OR GENE OVEREXPRESSION MUTATION

(71) Applicant: Repare Therapeutics Inc., Saint-Laurent (CA)

(72) Inventors: Jorge Sergio REIS, New York, NY (US); Maria KOEHLER, Narberth, PA (US); Victoria RIMKUNAS, Reading, MA (US); Dominik GLODZIK, Boston, MA (US); Michael ZINDA, Waltham, MA (US); Robert DABER, Farmingdale, NJ (US); Ian SILVERMAN, Arlington, MA (US)

(73) Assignee: Repare Therapeutics Inc., Saint-Laurent, QC (CA)

(21) Appl. No.: 18/288,308

(22) PCT Filed: Apr. 28, 2022
(86) PCT No.: PCT/CA2022/050655

§ 371 Date: Oct. 25, 2023

Related U.S. Application Data
(60) Provisional application No. 63/278,585, filed on Nov. 12, 2021.
(60) Provisional application No. 63/180,741, filed on Apr. 28, 2021.

(51) Int. Cl. C12Q 1/6886; A61K 31/497; A61K 31/519; A61K 31/5377; A61K 45/06; C12Q 1/6874

(52) U.S. Cl. CPC C12Q 1/6886; A61K 31/497; A61K 31/519; A61K 31/5377; A61K 45/06; C12Q 1/6874; C12Q 2600/156

(57) ABSTRACT

Methods of treating cancers having a biallelic ATM loss of function mutation are disclosed. Also disclosed are methods of inducing cell death in cancer cells having a biallelic ATM loss of function mutation. The methods typically include administering to the subject in need thereof an effective amount of an ATR inhibitor or contacting the cancer cell having a biallelic ATM loss of function mutation with an ATR inhibitor. Also disclosed are methods of treating cancers amplified for CCNE1.

### FIELD OF THE INVENTION

The invention relates to methods of treating cancers having a biallelic ATM loss of function using ATR inhibitors and methods of identifying a mutation as being biallelic or monoallelic, as well as being germline or somatic.

### BACKGROUND

ATR has been identified as an important cancer target since it is essential for dividing cells. ATR deficient mice are embryonic lethal, however, adult mice with conditional ATR knocked out are viable with effects on rapidly proliferating tissues and stem cell populations. Mouse embryonic stem cells lacking ATR will only divide for 1-2 doublings and then die. Interestingly, mice harboring hypomorphic ATR mutations that reduce expression of ATR to 10% of normal levels showed reduced H-rasG12D-induced tumor growth with minimal effects on proliferating normal cells, e.g., the bone marrow or intestinal epithelial cells.

There is a need for new anti-cancer therapeutic methods and, in particular, those targeting patient populations particularly susceptible to an anti-cancer therapy.

### SUMMARY OF THE INVENTION

In general, the invention provides methods of treating cancers (e.g., those having a biallelic ATM loss of function mutation or those having amplified CCNE1), inducing cell death in a cancer cell (e.g., that which has a biallelic ATM loss of function mutation or amplified CCNE1), or identifying a subject as having a biallelic loss of function for a target gene (e.g., ATM) or having an amplified gene (e.g., CCNE1).

In one aspect, the invention provides a method of treating a cancer having a biallelic ATM loss of function mutation in a subject, the method including administering to the subject in need thereof an effective amount of an ATR inhibitor.

In another aspect, the invention provides a method of treating a cancer in a subject, the method including administering to the subject in need thereof an effective amount of an ATR inhibitor, where the cancer has been previously identified as having a biallelic ATM loss of function mutation.

In yet another aspect, the invention provides a method of inducing a cell death in a cancer cell having a biallelic ATM loss of function mutation, the method including contacting the cell with an ATR inhibitor.

In some embodiments, the cell is in a subject.

In some embodiments, the method further includes identifying the cancer as having a biallelic ATM loss of function mutation prior to the administering step or contacting step.

In some embodiments, the identifying step includes the step of:
from read counts (e.g., obtained from next-generation sequencing) for a plurality of single nucleotide variants (SNVs) including homozygous and heterozygous SNVs obtained from sequencing a sample including the cancer cell and from reference read counts, determining an integer total copy number of a locus segment within an ATM gene region in a cancer cell from the subject or in the cancer cell and/or two integer allele-specific copy numbers of the locus segment,
where the cancer is identified as having a biallelic ATM loss of function mutation,
if at least one of the integer total copy number and the integer allele-specific copy numbers is 0, provided that the remaining ATM allele, if present, includes an inactivating mutation, or
if none of the integer allele-specific copy numbers is 0 and ATM alleles are present, each of the ATM alleles independently having an inactivating mutation.

In some embodiments, the determining step includes the steps of:
from the read counts and the reference read counts, determining total copy number log-ratios, allelic copy number log-odds ratios, and target coverage values for the SNVs;
segmenting the total copy number log-ratios and the allelic copy number log-odds ratios;
estimating sample purity and sample ploidy for the cancer cell from the total copy number log-ratios and the target coverage values; and
from the target coverage values, the sample purity, the sample ploidy, the total copy number log-ratios, and the allelic copy number log-odds ratios, generating an integer total copy number of a segment including a plurality of SNVs within an ATM gene region in the cancer cell and two integer allele-specific copy numbers of the segment.

In some embodiments, the method further includes the step of adjusting the ratios for location shift.

In some embodiments, the plurality of SNVs includes consistently covered SNVs (e.g., each of the consistently covered SNVs has the mean coverage of at least 200× reads across panel of normal samples). In some embodiments, each of the consistently covered SNVs has the mean coverage of at least 300× reads across panel of normal samples. In some embodiments, the plurality of SNVs includes frequent SNVs, the frequent SNVs having an allele frequency of 33% to 66% in humans. In some embodiments, the plurality of SNVs includes SNVs proximal to the frequent SNVs (e.g., disposed within 300 contiguous nucleobases downstream from the frequent SNV). In some embodiments, the plurality of SNVs includes SNVs, each of the SNVs having a 5′-flanking sequence of at least 20 contiguous nucleobases including 25-75% GC content, where the 5′-flanking sequence is unique and does not include other SNVs. In some embodiments, the plurality of SNVs includes at least 20 heterozygous SNVs. In some embodiments, the reference read counts are from a panel of normal samples. In some embodiments, the plurality of SNVs includes scaffold SNVs (e.g., scaffold SNVs may be useful to limit the solution space for the integer total copy number and integer allele-specific copy numbers). In some embodiments, the ATM gene region includes ATM and flanking regions up to 10 kilobases each. In some embodiments, the ATM gene region includes ATM and flanking regions up to 5 kilobases each. In some embodiments, the ATM gene region includes ATM and flanking regions up to 2 kilobases each. In some embodiments, the ATM gene region is an ATM exome region. In some embodiments, the ATM gene region is an ATM transcriptome region. In some embodiments, the ATM gene region is an ATM genome region. In some embodiments, the biallelic ATM loss of function mutation includes at least one somatic ATM loss of function mutation. In some embodiments, the biallelic ATM loss of function mutation includes at least one germline ATM loss of function mutation. In some embodiments, the biallelic ATM loss of function mutation includes one somatic ATM loss of function mutation and one germline ATM loss of function mutation.

In some embodiments, the cancer is listed in FIG. 15A. In some embodiments, the cancer is listed in FIG. 15B. In some embodiments, the cancer is lung adenocarcinoma. In some embodiments, the cancer is adrenocortical carcinoma. In some embodiments, the cancer is breast invasive carcinoma. In some embodiments, the cancer is LumB positive breast invasive carcinoma. In some embodiments, the cancer is Her2 positive breast invasive carcinoma. In some embodiments, the cancer is basal-like breast invasive carcinoma. In some embodiments, the cancer is pancreatic adenocarcinoma. In some embodiments, the cancer is bladder urothelial carcinoma. In some embodiments, the cancer is rectum adenocarcinoma. In some embodiments, the cancer is stomach adenocarcinoma. In some embodiments, the cancer is skin cutaneous melanoma. In some embodiments, the cancer is colon adenocarcinoma. In some embodiments, the cancer is prostate adenocarcinoma. In some embodiments, the cancer is glioblastoma multiforme. In some embodiments, the cancer is esophageal carcinoma. In some embodiments, the cancer is uterine corpus endometrial carcinoma. In some embodiments, the cancer is liver hepatocellular carcinoma. In some embodiments, the cancer is uterine corpus endometrial carcinoma. In some embodiments, the cancer is lung squamous cell carcinoma. In some embodiments, the cancer is a sarcoma. In some embodiments, the cancer is ovarian serous cystadenocarcinoma. In some embodiments, the ATR inhibitor is a compound of formula (I):

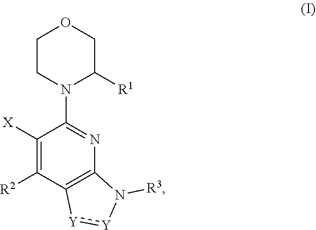

or a pharmaceutically acceptable salt thereof,
where
   [symbol] is a double bond, and each Y is independently N or CR⁴; or [symbol] is a single bond, and each Y is independently NR^{Y}, carbonyl, or C(R^{Y})₂; where each R^{Y} is independently H or optionally substituted C₁₋₆ alkyl;
   R¹ is optionally substituted C₁₋₆ alkyl or H;
   R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
   R³ is optionally substituted C₁₋₉ heteroaryl or optionally substituted C₁₋₉ heteroaryl C₁₋₆ alkyl;
   each R⁴ is independently hydrogen, halogen, optionally substituted C₁₋₆ alkyl, optionally substituted C₂₋₆ alkenyl, or optionally substituted C₂₋₆ alkynyl;
   each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
   each R^{5A} is independently optionally substituted C₁₋₆ alkyl, optionally substituted C₃₋₈ cycloalkyl, or optionally substituted C₆₋₁₀ aryl;
   R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
   each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
   Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
   X is hydrogen or halogen.

In some embodiments, the ATR inhibitor is a compound of formula (II):

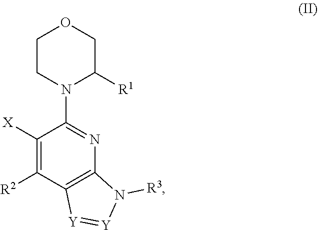

or a pharmaceutically acceptable salt thereof,
where
   each Y is independently N or CR⁴;
   R¹ is optionally substituted C₁₋₆ alkyl or H;
   R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
   R³ is optionally substituted C₁₋₉ heteroaryl or optionally substituted C₁₋₉ heteroaryl C₁₋₆ alkyl;
   each R⁴ is independently hydrogen, halogen, optionally substituted C₁₋₆ alkyl, optionally substituted C₂₋₆ alkenyl, or optionally substituted C₂₋₆ alkynyl;
   each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
   each R^{5A} is independently optionally substituted C₁₋₆ alkyl, optionally substituted C₃₋₈ cycloalkyl, or optionally substituted C₆₋₁₀ aryl;
   R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
   each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
   Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
   X is hydrogen or halogen.

In some embodiments, the ATR inhibitor is selected from the group consisting of compounds 43, 57, 62, 87, 93, 94, 95, 99, 100, 106, 107, 108, 109, 111, 112, 113, 114, 115, 116, 118, 119, 120, 121, 122, 123, 135, 147, 148, and pharmaceutically acceptable salts thereof from Table 1. In some embodiments, the ATR inhibitor is compound 43 or a pharmaceutically acceptable salt thereof from Table 1. In some embodiments, the ATR inhibitor is compound 121 or a pharmaceutically acceptable salt thereof from Table 1. In some embodiments, the ATR inhibitor is compound 122 or a pharmaceutically acceptable salt thereof from Table 1.

In some embodiments, the ATR inhibitor is

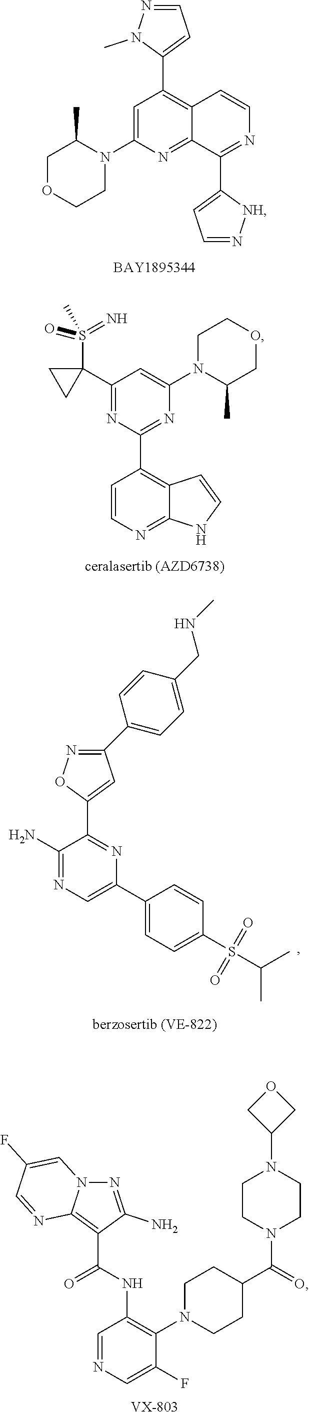

or a pharmaceutically acceptable salt thereof.

In still another aspect, the invention provides a method of identifying a cell from a subject as having a biallelic mutation in a target gene, the method including the step of:
from read counts for a plurality of single nucleotide variants (SNVs) (e.g., consistently covered SNVs) including homozygous and heterozygous SNVs (e.g., homozygous and heterozygous consistently covered SNVs) obtained from sequencing a sample including the cell and from reference read counts, determining an integer total copy number of a locus segment within a target gene region in the cell from the subject and/or two integer allele-specific copy numbers of the locus segment, the target gene region including the mutation, where the reference read counts are from a reference population of normal subjects,
where the cell is identified as having a biallelic mutation for a target gene,
if at least one of the integer total copy number and the integer allele-specific copy numbers is 0, provided that the remaining target gene allele, if present, includes the mutation, or
if none of the integer allele-specific copy numbers is 0 and target gene alleles are present, each of the target gene alleles independently having the mutation.

In some embodiments, the determining step includes the steps of:
from the read counts (e.g., read counts for an alternative allele) and the reference read counts (e.g., read counts for the reference allele), determining total copy number log-ratios, allelic copy number log-odds ratios, and target coverage values for the SNVs;
segmenting the total copy number log-ratios and the allelic copy number log-odds ratios;
estimating sample purity and sample ploidy for the cell from the total copy number log-ratios and the target coverage values; and
from the target coverage values, the sample purity, the sample ploidy, the total copy number log-ratios, and the allelic copy number log-odds ratios, generating an integer total copy number of a segment including a plurality of SNVs within a target gene region in the cell and two integer allele-specific copy numbers of the segment.

In some embodiments, the method further includes the step of adjusting the ratios for location shift.

In yet another aspect, the invention provides a method of identifying a cell from a subject as amplified for a target gene, the method including:
from read counts for a plurality of consistently covered single nucleotide variants (SNVs) including homozygous and heterozygous consistently covered SNVs obtained from sequencing a sample including the cell and from reference read counts, determining a total copy number of a locus segment within a target gene region in the cell from the subject and a sample ploidy, the target gene region including the mutation, where the reference read counts are from a panel of normal samples, where the cell is identified as amplified for a target gene, if the total copy number is at least double the sample ploidy, or if the total copy number is greater than the sample ploidy by at least two.

In some embodiments, the determining step includes:
from the read counts and the reference read counts, determining total copy number log-ratios, and target coverage values for the SNVs;
segmenting the total copy number log-ratios;
estimating sample purity and the sample ploidy for the cell from the total copy number log-ratios and the target coverage values; and
from the target coverage values, the sample purity, the sample ploidy, and the total copy number log-ratios, generating the total copy number of a locus segment within a target gene region.

In some embodiments, the total copy number is a normalized total copy number. In some embodiments, the cell is identified as amplified for a target gene if the total copy number is at least double the sample ploidy. In some embodiments, the cell is identified as amplified for a target gene if the total copy number is at least triple the sample ploidy. In some embodiments, the total copy number is a normalized total copy number. In some embodiments, the cell is identified as amplified for a target gene if the total copy number is greater than the sample ploidy by at least two. In some embodiments, the cell is identified as amplified for a target gene if the total copy number is greater than the sample ploidy by at least four.

In some embodiments, the method further includes adjusting the ratios for location shift.

In some embodiments, the target gene is CCNE1.

In still another aspect, the invention provides a method of treating a cancer in a subject, the method including:
identifying the cancer as amplified for CCNE1 according to the method described herein, and
administering to the subject in need thereof a therapeutically effective amount of a membrane-associated tyrosine and threonine-specific cdc2 inhibitory kinase (Myt1) inhibitor.

In some embodiments, the Myt1 inhibitor is a compound of formula (III):

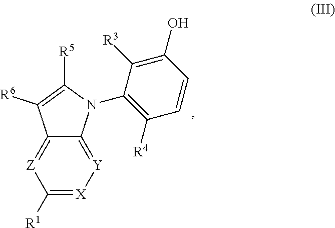

or a pharmaceutically acceptable salt thereof,
where
   each of X, Y, and Z is independently N or CR²;
   R¹ and each R² are independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, cyano, —N(R⁷)₂, —OR⁷, —C(O)N(R⁸)₂, —SO₂N(R⁸)₂, —SO₂R^{7A}, or -Q-R⁷⁸; or R¹ combines with one R² that is vicinal to R¹ to form an optionally substituted C₃₋₆ alkylene;
   each of R³ and R⁴ is independently optionally substituted C₁₋₆ alkyl or halogen;
   R⁵ is H or —N(R⁷)₂;
   R⁶ is —C(O)NH(R⁸), —C(O) R^{7A}, or —SO₂R^{7A};
   each R⁷ is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or —SO₂R^{7A}; or two R⁷ groups, together with the atom to which both are attached, combine to form an optionally substituted C₂₋₉ heterocyclyl;
   each R^{7A} is independently optionally substituted C₁₋₆ alkyl, optionally substituted C₃₋₈ cycloalkyl, or optionally substituted C₆₋₁₀ aryl;
   each R^{7B} is independently hydroxyl, optionally substituted C₁₋₆ alkyl, optionally substituted C₆₋₁₀ aryl, optionally substituted C₂₋₉ heterocyclyl, optionally substituted C₁₋₉ heteroaryl, —N(R⁷)₂, —C(O)N(R⁸)₂, —SO₂N(R⁸)₂, —SO₂R^{7A}, or optionally substituted alkoxy;
   each R⁸ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or two R⁸, together with the atom to which they are attached, combine to form an optionally substituted C₂₋₉ heterocyclyl;
   Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene optionally substituted C₆₋₁₀ arylene, optionally substituted C₂₋₉ heterocyclylene, or optionally substituted C₁₋₉ heteroarylene.

In some embodiments, the compound is enriched for the atropisomer of formula (IIIA):

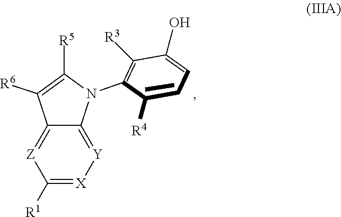

or a pharmaceutically acceptable salt thereof.

In some embodiments, X is CR².

In some embodiments, the compound is of formula (IV):

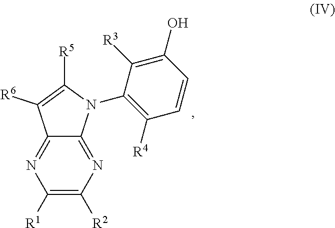

or a pharmaceutically acceptable salt thereof.

In some embodiments, the compound is enriched for the atropisomer of formula (IVA):

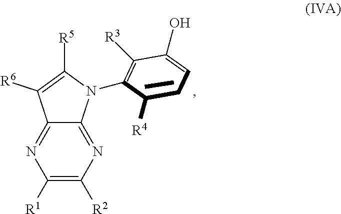

or a pharmaceutically acceptable salt thereof.

In some embodiments, the compound is of formula (V):

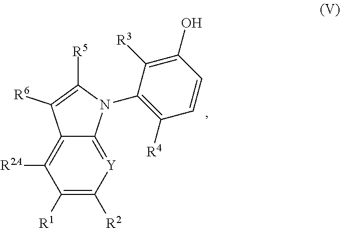

or a pharmaceutically acceptable salt thereof.
where R^{2A} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁷)₂, —OR⁷, —C(O) N(R⁸)₂, —SO₂N(R⁸)₂, —SO₂R^{7A}, or -Q-R^{7B}.

In some embodiments, the compound is enriched for the atropisomer of formula (VA):

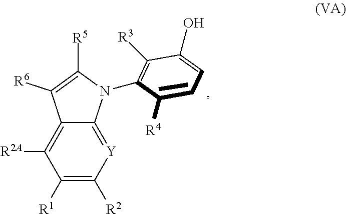

or a pharmaceutically acceptable salt thereof.

In some embodiments, the Myt1 inhibitor is any one of compounds 1-328 and pharmaceutically acceptable salts thereof from Table 2. In some embodiments, the Myt1 inhibitor is compound 181, or a pharmaceutically acceptable salt thereof, from Table 2. In some embodiments, the Myt1 inhibitor is compound 182, or a pharmaceutically acceptable salt thereof, from Table 2.

In some embodiments, the method further includes administering to the subject an effective amount of a WEE1 inhibitor, FEN1 inhibitor, TOP1 inhibitor, RRM1 inhibitor, RRM2 inhibitor, AURKB inhibitor, TOP2A inhibitor, ATR inhibitor, TTK inhibitor, SOD1 inhibitor, SOD2 inhibitor, BUB1 inhibitor, CDC7 inhibitor, SAE1 inhibitor, PLK1 inhibitor, UBA2 inhibitor, DUT inhibitor, HDAC3 inhibitor, CHEK1 inhibitor, AURKA inhibitor, MEN1 inhibitor, DOT1L inhibitor, CREBBP inhibitor, EZH2 inhibitor, PLK4 inhibitor, HASPIN inhibitor, METTL3 inhibitor, nucleoside analog, platinum-based DNA damaging agent, or a combination thereof.

In some embodiments, the method further includes adjusting the ratios for location shift. In some embodiments, the plurality of SNVs includes consistently covered SNVs. In some embodiments, each of the consistently covered SNVs has the mean coverage of at least 200× reads across panel of normal samples. In some embodiments, the plurality of SNVs includes frequent SNVs, the frequent SNVs having an allele frequency of 33% to 66% in humans. In some embodiments, the plurality of SNVs includes SNVs proximal to the frequent SNVs.

In some embodiments, the plurality of SNVs includes SNVs, each of the SNVs having a 5′-flanking sequence of at least 20 contiguous nucleobases including 25-75% GC content, where the 5′-flanking sequence is unique and does not include other SNVs. In some embodiments, the plurality of SNVs includes at least 20 heterozygous SNVs. In some embodiments, the reference read counts are from a panel of normal samples.

In some embodiments, the target gene region includes the target gene and flanking regions up to 10 kilobases each. In some embodiments, the target gene region includes the target gene and flanking regions up to 5 kilobases each. In some embodiments, the target gene region includes the target gene and flanking regions up to 2 kilobases each.

In some embodiments, the target gene region is a target gene exome region. In some embodiments, the target gene region is a target gene transcriptome region. In some embodiments, the target gene region is a target gene genome region.

In a further aspect, the invention provides a method of identifying a target mutation in a cell from a subject as being germline or somatic, the method including the steps of:
from read counts for a plurality of consistently covered single nucleotide variants (SNVs) including homozygous and heterozygous consistently covered SNVs obtained from sequencing a sample including the cell and from reference read counts, determining an observed allele fraction of a locus segment within a target gene region in the cell from the subject, the target gene region including the target mutation;
determining expected allele fractions for a germline target mutation and for a somatic target mutation;
comparing the observed allele fraction to the expected allele fractions to identify the most probable of the germline and somatic mutations; and
identifying the target mutation as germline or somatic as that which is the most probable for the germline and somatic mutations.

In yet further aspect, the invention provides a method of identifying a target mutation in a cell from a subject as being germline or somatic, the method including identifying the target mutation in the normal, matched sample from the subject,
where
if the target mutation present in the cell from the subject is identified in the normal, matched sample, the target mutation is germline, and
if the target mutation present in the cell from the subject is not identified in the normal, matched sample, the target mutation is somatic.

In some embodiments of any of the aspects, the comparing step is performed using Bayesian model comparison. In some embodiments of any of the aspects, each of the consistently covered SNVs has the mean coverage of at least 200× reads across panel of normal samples. In some embodiments of any of the aspects, the plurality of SNVs includes SNVs with an allele frequency of 33% to 66% in humans. In some embodiments, the plurality of SNVs includes SNVs proximal to the frequent SNVs (e.g., disposed within 300 contiguous nucleobases downstream from the frequent SNV). In some embodiments, the plurality of SNVs includes SNVs, each of the SNVs having a 5′-flanking sequence of at least 20 contiguous nucleobases including 25-75% GC content, where the 5′-flanking sequence is unique and does not include other SNVs. In some embodiments of any of the aspects, the plurality of SNVs includes at least 20 heterozygous SNVs. In some embodiments of any of the aspects, the plurality of SNVs includes scaffold SNVs (e.g., scaffold SNVs may be useful to limit the solution space for the integer total copy number and integer allele-specific copy numbers). In some embodiments of any of the aspects, the target gene region includes the target gene and flanking regions up to 10 kilobases each. In some embodiments of any of the aspects, the target gene region includes the target gene and flanking regions up to 5 kilobases each. In some embodiments of any of the aspects, the target gene region includes the target gene and flanking regions up to 2 kilobases each. In some embodiments of any of the aspects, the target gene region is a target exome region. In some embodiments of any of the aspects, the target gene region is a target transcriptome region. In some embodiments of any of the aspects, the target gene region is a target genome region. In some embodiments of any of the aspects, the cell from the subject is a cancer cell from the subject. In some embodiments of any of the aspects, the mutation is a germline mutation.

### Definitions

The term “acyl,” as used herein, represents a group-C(═O)—R, where R is alkyl, alkenyl, alkynyl, cycloalkyl, cycloalkenyl, cycloalkynyl, aryl, heteroaryl, or heterocyclyl. Acyl may be optionally substituted as described herein for each respective R group.

The term “alkanoyl,” as used herein, represents a hydrogen or an alkyl group that is attached to the parent molecular group through a carbonyl group and is exemplified by formyl (i.e., a carboxyaldehyde group), acetyl, propionyl, butyryl, and iso-butyryl. Unsubstituted alkanoyl groups contain from 1 to 7 carbons. The alkanoyl group may be unsubstituted of substituted (e.g., optionally substituted C1-7 alkanoyl) as described herein for alkyl group. The ending “-oyl” may be added to another group defined herein, e.g., aryl, cycloalkyl, and heterocyclyl, to define “aryloyl,” “cycloalkanoyl,” and “(heterocyclyl)oyl.” These groups represent a carbonyl group substituted by aryl, cycloalkyl, or heterocyclyl, respectively. Each of “aryloyl,” “cycloalkanoyl,” and “(heterocyclyl)oyl” may be optionally substituted as defined for “aryl,” “cycloalkyl,” or “heterocyclyl,” respectively.

The term “alkenyl,” as used herein, represents acyclic monovalent straight or branched chain hydrocarbon groups of containing one, two, or three carbon-carbon double bonds. Non-limiting examples of the alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted as defined herein for alkyl.

The term “alkoxy,” as used herein, represents a chemical substituent of formula —OR, where R is a C₁₋₆ alkyl group, unless otherwise specified. In some embodiments, the alkyl group can be further substituted as defined herein. The term “alkoxy” can be combined with other terms defined herein, e.g., aryl, cycloalkyl, or heterocyclyl, to define an “aryl alkoxy,” “cycloalkyl alkoxy,” and “(heterocyclyl)alkoxy” groups. These groups represent an alkoxy that is substituted by aryl, cycloalkyl, or heterocyclyl, respectively. Each of “aryl alkoxy,” “cycloalkyl alkoxy,” and “(heterocyclyl)alkoxy” may optionally substituted as defined herein for each individual portion.

The term “alkoxyalkyl,” as used herein, represents a chemical substituent of formula-L-O—R, where L is C₁₋₆ alkylene, and R is C₁₋₆ alkyl. An optionally substituted alkoxyalkyl is an alkoxyalkyl that is optionally substituted as described herein for alkyl.

The term “alkyl,” as used herein, refers to an acyclic straight or branched chain saturated hydrocarbon group, which, when unsubstituted, has from 1 to 12 carbons, unless otherwise specified. In certain preferred embodiments, unsubstituted alkyl has from 1 to 6 carbons. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: amino; aryl; aryloxy; azido; cycloalkyl; cycloalkoxy; cycloalkenyl; cycloalkynyl; halo; heterocyclyl; (heterocyclyl)oxy; heteroaryl; hydroxy; nitro; thiol; silyl; cyano; alkylsulfonyl; alkylsulfinyl; alkylsulfenyl; ═O; ═S; —SO₂R, where R is amino or cycloalkyl; ═NR′, where R′ is H, alkyl, aryl, or heterocyclyl. Each of the substituents may itself be unsubstituted or, valency permitting, substituted with unsubstituted substituent(s) defined herein for each respective group.

The term “alkylene,” as used herein, refers to a divalent alkyl group. An optionally substituted alkylene is an alkylene that is optionally substituted as described herein for alkyl.

The term “alkylamino,” as used herein, refers to a group having the formula —N(R^{N1})₂ or —NHR^{N1}, in which R^{N1} is alkyl, as defined herein. The alkyl portion of alkylamino can be optionally substituted as defined for alkyl. Each optional substituent on the substituted alkylamino may itself be unsubstituted or, valency permitting, substituted with unsubstituted substituent(s) defined herein for each respective group.

The term “alkylsulfenyl,” as used herein, represents a group of formula —S-(alkyl). Alkylsulfenyl may be optionally substituted as defined for alkyl.

The term “alkylsulfinyl,” as used herein, represents a group of formula —S(O)-(alkyl). Alkylsulfinyl may be optionally substituted as defined for alkyl.

The term “alkylsulfonyl,” as used herein, represents a group of formula —S(O)₂-(alkyl). Alkylsulfonyl may be optionally substituted as defined for alkyl.

The term “alkynyl,” as used herein, represents monovalent straight or branched chain hydrocarbon groups of from two to six carbon atoms containing at least one carbon-carbon triple bond and is exemplified by ethynyl, 1-propynyl, and the like. The alkynyl groups may be unsubstituted or substituted (e.g., optionally substituted alkynyl) as defined for alkyl.

The term “allele fraction,” as used herein, refers to a normalized measure of the allelic intensity ratio of a variant allele, such that an allele fraction of 1 or 0 indicates the complete absence of one of the two alleles. For ploidy of 2, an allele fraction of 0.5 indicates the equal presence of both alleles. For ploidy of 3, an allele fraction of 0.33 or 0.66 indicates the presence of one copy of one allele and two copies of another allele. For ploidy of 4, an allele fraction of 0.25 or 0.75 indicates the presence of one copy of one allele and three copies of another allele, and an allele fraction of 0.5 indicates the equal presence of both alleles. An allele fraction can be measured as a B Allele Frequency.

The term “allelic copy number log-odds ratio,” as used herein, refers to a ratio of parental copy numbers in a cancer cell (E[log OR]=[p1·ϕ+(1−ϕ)]/[p2·ϕ+(1−ϕ]), where E[log OR] is the expected value of log OR, p1 is a parental copy number of the variant allele, p2 is a parental copy number of the allele from the other parent, and ϕ is a cellular fraction that is a function of tumor purity and clonal frequency (for subclonal alterations).

The term “amino,” as used herein, represents —N(R^{N1})₂, where, if amino is unsubstituted, both R^{N1} are H; or, if amino is substituted, each R^{N1} is independently H, —OH, —NO₂, —N(R^{N2})₂, —SO₂OR^{N2}, —SO₂R^{N2}, —SOR^{N2}, —COOR^{N2}, an N-protecting group, alkyl, alkenyl, alkynyl, alkoxy, aryl, arylalkyl, aryloxy, cycloalkyl, cycloalkenyl, heteroalkyl, or heterocyclyl, provided that at least one R^{N1} is not H, and where each R^{N2} is independently H, alkyl, or aryl. Each of the substituents may itself be unsubstituted or substituted with unsubstituted substituent(s) defined herein for each respective group. In some embodiments, amino is unsubstituted amino (i.e., —NH₂) or substituted amino (e.g., NHR^{N1}), where R^{N1} is independently —OH, SO₂OR^{N2}, —SO₂R^{N2}, —SOR^{N2}, —COOR^{N2}, optionally substituted alkyl, or optionally substituted aryl, and each R^{N2} can be optionally substituted alkyl or optionally substituted aryl. In some embodiments, substituted amino may be alkylamino, in which the alkyl groups are optionally substituted as described herein for alkyl. In some embodiments, an amino group is —NHR^{N1}, in which R^{N1} is optionally substituted alkyl.

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; and cyano. Each of the substituents may itself be unsubstituted or substituted with unsubstituted substituent(s) defined herein for each respective group.

The term “aryl alkyl,” as used herein, represents an alkyl group substituted with an aryl group. The aryl and alkyl portions may be optionally substituted as the individual groups as described herein.

The term “arylene,” as used herein, refers to a divalent aryl group. An optionally substituted arylene is an arylene that is optionally substituted as described herein for aryl.

The term “aryloxy,” as used herein, represents a chemical substituent of formula —OR, where R is an aryl group, unless otherwise specified. In optionally substituted aryloxy, the aryl group is optionally substituted as described herein for aryl.

The term “ATM,” as used herein, represents ATM serine/threonine kinase.

The term “ATR inhibitor,” as used herein, represents a compound that upon contacting the enzyme ATR kinase, whether in vitro, in cell culture, or in an animal, reduces the activity of ATR kinase, such that the measured ATR kinase IC₅₀ is 10 μM or less (e.g., 5 μM or less or 1 μM or less). For certain ATR inhibitors, the ATR kinase IC₅₀ may be 100 nM or less (e.g., 10 nM or less, or 1 nM or less) and could be as low as 100 pM or 10 pM. Preferably, the ATR kinase IC₅₀ is 0.1 nM to 1 μM (e.g., 0.1 nM to 750 nM, 0.1 nM to 500 nM, or 0.1 nM to 250 nM).

The term “ATR kinase,” as used herein, refers to Ataxia-telangiectasia and RAD-3-related protein kinase.

The term “azido,” as used herein, represents an —N₃ group.

The term “biallelic loss of function mutation,” as used herein, refers to a mutation within a subject's cell (e.g., cancer cell) that results in the elimination of the active form of a target gene in the cell. For example, a “biallelic ATM loss of function mutation” refers to a mutation within a subject's cell (e.g., cancer cell) that results in the elimination of the active form of ATM gene in the cell.

The term “carbocyclic,” as used herein, represents an optionally substituted C3-16 monocyclic, bicyclic, or tricyclic structure in which the rings, which may be aromatic or non-aromatic, are formed by carbon atoms. Carbocyclic structures include cycloalkyl, cycloalkenyl, cycloalkynyl, and certain aryl groups.

The term “carbonyl,” as used herein, represents a —C(O)— group.

The term “cyano,” as used herein, represents —CN group.

The term “cycloalkenyl,” as used herein, refers to a non-aromatic carbocyclic group having at least one double bond in the ring and from three to ten carbons (e.g., a C₃₋₁₀ cycloalkenyl), unless otherwise specified. Non-limiting examples of cycloalkenyl include cycloprop-1-enyl, cycloprop-2-enyl, cyclobut-1-enyl, cyclobut-1-enyl, cyclobut-2-enyl, cyclopent-1-enyl, cyclopent-2-enyl, cyclopent-3-enyl, norbornen-1-yl, norbornen-2-yl, norbornen-5-yl, and norbornen-7-yl. The cycloalkenyl group may be unsubstituted or substituted (e.g., optionally substituted cycloalkenyl) as described for cycloalkyl.

The term “cycloalkenyl alkyl,” as used herein, represents an alkyl group substituted with a cycloalkenyl group, each as defined herein. The cycloalkenyl and alkyl portions may be substituted as the individual groups defined herein.

The term “cycloalkoxy,” as used herein, represents a chemical substituent of formula —OR, where R is cycloalkyl group, unless otherwise specified. In some embodiments, the cycloalkyl group can be further substituted as defined herein.

The term “cycloalkyl,” as used herein, refers to a cyclic alkyl group having from three to ten carbons (e.g., a C₃-C₁₀ cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be unsubstituted or substituted (e.g., optionally substituted cycloalkyl) with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; heteroaryl; hydroxy; nitro; thiol; silyl; cyano; ═O; ═S; —SO₂R, where R is amino or cycloalkyl; ═NR′, where R′ is H, alkyl, aryl, or heterocyclyl; or —CON(R^{A})₂, where each R^{A} is independently H or alkyl, or both R^{A}, together with the atom to which they are attached, combine to form heterocyclyl. Each of the substituents may itself be unsubstituted or substituted with unsubstituted substituent(s) defined herein for each respective group.

The term “cycloalkyl alkyl,” as used herein, represents an alkyl group substituted with a cycloalkyl group, each as defined herein. The cycloalkyl and alkyl portions may be optionally substituted as the individual groups described herein.

The term “cycloalkylene,” as used herein, represents a divalent cycloalkyl group. An optionally substituted cycloalkylene is a cycloalkylene that is optionally substituted as described herein for cycloalkyl.

The term “cycloalkynyl,” as used herein, refers to a monovalent carbocyclic group having one or two carbon-carbon triple bonds and having from eight to twelve carbons, unless otherwise specified. Cycloalkynyl may include one transannular bond or bridge. Non-limiting examples of cycloalkynyl include cyclooctynyl, cyclononynyl, cyclodecynyl, and cyclodecadiynyl. The cycloalkynyl group may be unsubstituted or substituted (e.g., optionally substituted cycloalkynyl) as defined for cycloalkyl.

“Disease” or “condition” refer to a state of being or health status of a patient or subject capable of being treated with the compounds or methods provided herein.

The term “halo,” as used herein, represents a halogen selected from bromine, chlorine, iodine, and fluorine.

The term “heteroalkyl,” as used herein refers to an alkyl, alkenyl, or alkynyl group interrupted once by one or two heteroatoms; twice, each time, independently, by one or two heteroatoms; three times, each time, independently, by one or two heteroatoms; or four times, each time, independently, by one or two heteroatoms. Each heteroatom is, independently, O, N, or S. In some embodiments, the heteroatom is O or N. None of the heteroalkyl groups includes two contiguous oxygen or sulfur atoms. The heteroalkyl group may be unsubstituted or substituted (e.g., optionally substituted heteroalkyl). When heteroalkyl is substituted and the substituent is bonded to the heteroatom, the substituent is selected according to the nature and valency of the heteroatom. Thus, the substituent bonded to the heteroatom, valency permitting, is selected from the group consisting of ═O, —N(R^{N2})₂, —SO₂OR^{N3}, —SO₂R^{N2}, —SOR^{N3}, —COOR^{N3}, an N protecting group, alkyl, alkenyl, alkynyl, aryl, cycloalkyl, cycloalkenyl, cycloalkynyl, heterocyclyl, or cyano, where each R^{N2} is independently H, alkyl, cycloalkyl, cycloalkenyl, cycloalkynyl, aryl, or heterocyclyl, and each R^{N3} is independently alkyl, cycloalkyl, cycloalkenyl, cycloalkynyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or I. It is understood that carbon atoms are found at the termini of a heteroalkyl group.

The term “heteroaryl alkyl,” as used herein, represents an alkyl group substituted with a heteroaryl group, each as defined herein. The heteroaryl and alkyl portions may be optionally substituted as the individual groups described herein.

The term “heteroarylene,” as used herein, represents a divalent heteroaryl. An optionally substituted heteroarylene is a heteroarylene that is optionally substituted as described herein for heteroaryl.

The term “heteroaryloxy,” as used herein, refers to a structure —OR, in which R is heteroaryl. Heteroaryloxy can be optionally substituted as defined for heterocyclyl.

The term “heterocyclyl,” as used herein, represents a monocyclic, bicyclic, tricyclic, or tetracyclic ring system having fused, bridging, and/or spiro 3-, 4-, 5-, 6-, 7-, or 8-membered rings, unless otherwise specified, containing one, two, three, or four heteroatoms independently selected from the group consisting of nitrogen, oxygen, and sulfur. In some embodiments, “heterocyclyl” is a monocyclic, bicyclic, tricyclic, or tetracyclic ring system having fused or bridging 5-, 6-, 7-, or 8-membered rings, unless otherwise specified, containing one, two, three, or four heteroatoms independently selected from the group consisting of nitrogen, oxygen, and sulfur. Heterocyclyl can be aromatic or non-aromatic. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups include from 1 to 16 carbon atoms unless otherwise specified. Certain heterocyclyl groups may include up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, etc. If the heterocyclic ring system has at least one aromatic resonance structure or at least one aromatic tautomer, such structure is an aromatic heterocyclyl (i.e., heteroaryl). Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl, tetrazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four, five, or six substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; cyano; ═O; ═S; ═NR′, where R′ is H, alkyl, aryl, or heterocyclyl. Each of the substituents may itself be unsubstituted or substituted with unsubstituted substituent(s) defined herein for each respective group.

The term “heterocyclyl alkyl,” as used herein, represents an alkyl group substituted with a heterocyclyl group, each as defined herein. The heterocyclyl and alkyl portions may be optionally substituted as the individual groups described herein.

The term “heterocyclylene,” as used herein, represents a divalent heterocyclyl. An optionally substituted heterocyclylene is a heterocyclylene that is optionally substituted as described herein for heterocyclyl.

The term “(heterocyclyl)oxy,” as used herein, represents a chemical substituent of formula —OR, where R is a heterocyclyl group, unless otherwise specified. (Heterocyclyl)oxy can be optionally substituted in a manner described for heterocyclyl.

The terms “hydroxyl” and “hydroxy,” as used interchangeably herein, represent an —OH group.

The term “isotopically enriched,” as used herein, refers to the pharmaceutically active agent with the isotopic content for one isotope at a predetermined position within a molecule that is at least 100 times greater than the natural abundance of this isotope. For example, a composition that is isotopically enriched for deuterium includes an active agent with at least one hydrogen atom position having at least 100 times greater abundance of deuterium than the natural abundance of deuterium. Preferably, an isotopic enrichment for deuterium is at least 1000 times greater than the natural abundance of deuterium. More preferably, an isotopic enrichment for deuterium is at least 4000 times greater (e.g., at least 4750 times greater, e.g., up to 5000 times greater) than the natural abundance of deuterium.

The term “Myt1,” as used herein, refers to membrane-associated tyrosine and threonine-specific cdc2-inhibitory kinase (Myt1) (Gene name PKMYT1).

The term “Myt1 inhibitor,” as used herein, represents a compound that upon contacting the enzyme Myt1, whether in vitro, in cell culture, or in an animal, reduces the activity of Myt1, such that the measured Myt1 IC₅₀ is 10 μM or less (e.g., 5 μM or less or 1 μM or less). For certain Myt1 inhibitors, the Myt1 IC₅₀ may be 100 nM or less (e.g., 10 nM or less, or 3 nM or less) and could be as low as 100 μM or 10 μM. Preferably, the Myt1 IC₅₀ is 1 nM to 1 μM (e.g., 1 nM to 750 nM, 1 nM to 500 nM, or 1 nM to 250 nM). Even more preferably, the Myt1 IC₅₀ is less than 20 nm (e.g., 1 nM to 20 nM).

The term “Next Generation Sequencing (NGS)” herein refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation.

The term “nitro,” as used herein, represents an —NO₂ group.

The term “oxo,” as used herein, represents a divalent oxygen atom (e.g., the structure of oxo may be shown as ═O).

The term “Ph,” as used herein, represents phenyl.

The term “pharmaceutical composition,” as used herein, represents a composition containing a compound described herein, formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

The term “pharmaceutically acceptable excipient” or “pharmaceutically acceptable carrier,” as used interchangeably herein, refers to any ingredient other than the compounds described herein (e.g., a vehicle capable of suspending or dissolving the active compound) and having the properties of being nontoxic and non-inflammatory in a patient. Excipients may include, for example: antiadherente, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Excipients include, e.g., butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “protecting group,” as used herein, represents a group intended to protect a hydroxy, an amino, or a carbonyl from participating in one or more undesirable reactions during chemical synthesis. The term “O-protecting group,” as used herein, represents a group intended to protect a hydroxy or carbonyl group from participating in one or more undesirable reactions during chemical synthesis. The term “N-protecting group,” as used herein, represents a group intended to protect a nitrogen containing (e.g., an amino, amido, heterocyclic N—H, or hydrazine) group from participating in one or more undesirable reactions during chemical synthesis. Commonly used O- and N-protecting groups are disclosed in Greene, “Protective Groups in Organic Synthesis,” 3rd Edition (John Wiley & Sons, New York, 1999), which is incorporated herein by reference. O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups are substituted alkyl, aryl, and aryl-alkyl ethers (e.g., trityl; methylthiomethyl; methoxymethyl; benzyloxymethyl; siloxymethyl; 2,2,2, -trichloroethoxymethyl; tetrahydropyranyl; tetrahydrofuranyl; ethoxyethyl; 1-[2-(trimethylsilyl) ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p methoxybenzyloxycarbonyl, p-nitrobenzyloxycarbonyl, 2-nitrobenzyloxycarbonyl, p bromobenzyloxycarbonyl, 3,4-dimethoxybenzyloxycarbonyl, 3,5 dimethoxybenzyl oxycarbonyl, 2,4-dimethoxybenzyloxycarbonyl, 4 methoxybenzyloxycarbonyl, 2-nitro-4,5-dimethoxybenzyloxycarbonyl, 3,4,5 trimethoxybenzyloxycarbonyl, 1-(p-biphenylyl)-1-methylethoxycarbonyl, α,α-dimethyl-3,5 dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2, -trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, aryl-alkyl groups such as benzyl, p-methoxybenzyl, 2,4-dimethoxybenzyl, triphenylmethyl, benzyloxymethyl, and the like, silylalkylacetal groups such as [2-(trimethylsilyl) ethoxy]methyl and silyl groups such as trimethylsilyl, and the like. Useful N-protecting groups are formyl, acetyl, benzoyl, pivaloyl, t-butylacetyl, alanyl, phenylsulfonyl, benzyl, dimethoxybenzyl, [2-(trimethylsilyl) ethoxy]methyl (SEM), tetrahydropyranyl (THP), t-butyloxycarbonyl (Boc), and benzyloxycarbonyl (Cbz).

The term “purity,” when used herein in reference to methods including the loss-of-function identification, refers to the proportion of target cells (e.g., cancer cells) relative to all cells in the samples.

The term “sarcoma” generally refers to a tumor which is made up of a substance like the embryonic connective tissue and is generally composed of closely packed cells embedded in a fibrillar or homogeneous substance. Non-limiting examples of sarcomas that may be treated with a compound or method provided herein include, e.g., a chondrosarcoma, fibrosarcoma, lymphosarcoma, melanosarcoma, myxosarcoma, osteosarcoma, Abernethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “scaffold SNV,” as used herein, represent frequent, well-covered single nucleotide variants outside the target gene region and spaced throughout the chromosome carrying the target gene region.

The term “subject,” as used herein, represents a human or non-human animal (e.g., a mammal) that is suffering from, or is at risk of, disease or condition, as determined by a qualified professional (e.g., a doctor or a nurse practitioner) with or without known in the art laboratory test(s) of sample(s) from the subject. Preferably, the subject is a human. Non-limiting examples of diseases and conditions include diseases having the symptom of cell hyperproliferation, e.g., a cancer.

The term “target coverage,” as used herein, refers to the average number of reads aligning to a chromosomal position in a target region.

The term “tautomer” refers to structural isomers that readily interconvert, often by relocation of a proton. Tautomers are distinct chemical species that can be identified by differing spectroscopic characteristics, but generally cannot be isolated individually. Non-limiting examples of tautomers include ketone-enol, enamine-imine, amide-imidic acid, nitroso-oxime, ketene-ynol, and amino acid-ammonium carboxylate.

The term “therapeutically effective amount,” as used herein, means the amount of an ATR inhibitor sufficient to treat cancer.

The term “total copy number log-ratio,” as used herein, refers to a cancer cell over control cell signal ratio. The total copy number log-ratio deviations from an average of 0 for a given region suggest signal intensity to be higher (if greater than 0) or lower (if less than 0) than expected for two chromosomal copies. The total copy number log-ratio, also known as Log R, may be estimated using GenomeStudio® software from Illumina.

“Treatment” and “treating,” as used herein, refer to the medical management of a subject with the intent to improve, ameliorate, stabilize, prevent or cure a disease or condition. This term includes active treatment (treatment directed to improve the disease or condition); causal treatment (treatment directed to the cause of the associated disease or condition); palliative treatment (treatment designed for the relief of symptoms of the disease or condition); preventative treatment (treatment directed to minimizing or partially or completely inhibiting the development of the associated disease or condition); and supportive treatment (treatment employed to supplement another therapy). A disease or condition may be a cancer.

### BRIEF DESCRIPTION OF THE DRAWINGS

FIGS. 1A-1C illustrates three mechanisms of germline ATM loss of function mutations. FIGS. 1A and 1C illustrate monoallelic germline ATM loss of function mutations, and FIG. 1B illustrates a biallelic germline ATM loss of function mutation.

FIG. 2A is a chart showing the copy number calls across all chromosomes as determined by whole genome sequencing (WGS) for a female subject, age 46, with papillary serous carcinoma.

FIG. 2B is a chart showing the copy number calls across all chromosomes as determined using a single nucleotide variant (SNV) Panel Version 1 for a female subject, age 46, with papillary serous carcinoma.

FIG. 3A is a chart showing the copy number calls across all chromosomes as determined by WGS for a female subject, age 62, with pancreatic ductal adenocarcinoma.

FIG. 3B is a chart showing the copy number calls across all chromosomes as determined using an SNV Panel Version 1 for a female subject, age 62, with pancreatic ductal adenocarcinoma.

FIG. 4A is a chart showing the copy number calls across all chromosomes as determined by WGS for a female subject, age 72, with metastatic breast carcinoma (ER-, PR—, Her2-).

FIG. 4B is a chart showing the copy number calls across all chromosomes as determined using an SNV Panel Version 1 for a female subject, age 72, with metastatic breast carcinoma (ER-PR—, Her2-).

FIG. 5A is a chart showing the single nucleotide variant (SNV) coverage of SNV Panel Version 1 on chromosome 1.

FIG. 5B is a chart showing the SNV coverage of SNV Panel Version 2 on chromosome 1.

FIG. 6 is a series of charts showing the sequencing coverage depth (top graph), b-allele fraction (middle graph), and copy number profile (bottom graph) for a cancer biopsy from a subject having a biallelic germline ATM loss of function mutation.

FIG. 7A is a chart showing the copy number calls across all chromosomes as determined by WGS for a subject, age 55, with lung adenocarcinoma.

FIG. 7B is a chart showing the copy number calls across all chromosomes as determined using an SNV Panel Version 2 for a subject, age 55, with lung adenocarcinoma.

FIG. 8A is a chart showing the copy number calls across all chromosomes as determined by WGS for a subject, age 72, with breast cancer, lum B.

FIG. 8B is a chart showing the copy number calls across all chromosomes as determined using an SNV Panel Version 2 for a subject, age 72, with breast cancer, lum B.

FIG. 9A is a chart showing the copy number calls across all chromosomes as determined by WGS for a subject, age 41, with bladder cancer.

FIG. 9B is a chart showing the copy number calls across all chromosomes as determined using an SNV Panel Version 2 for a subject, age 41, with bladder cancer.

FIG. 10A is a chart showing the copy number calls across all chromosomes as determined by WGS for a subject, age 80, with bladder cancer.

FIG. 10B is a chart showing the copy number calls across all chromosomes as determined using an SNV Panel Version 2 for a subject, age 80, with bladder cancer.

FIG. 11A is a chart showing the copy number calls across all chromosomes as determined by WGS for a subject, age 66, with breast luminal B cancer.

FIG. 11B is a chart showing the copy number calls across all chromosomes as determined using an SNV Panel Version 2 for a subject, age 66, with breast luminal B cancer.

FIG. 12A is a chart showing the copy number calls across all chromosomes as determined by WGS for a subject, age 62, with prostate cancer.

FIG. 12B is a chart showing the copy number calls across all chromosomes as determined using an SNV Panel Version 2 for a subject, age 62, with prostate cancer.

FIG. 13A is a chart showing the copy number calls across all chromosomes as determined by WGS for a subject, age 69, with uterus cancer.

FIG. 13B is a chart showing the copy number calls across all chromosomes as determined using an SNV Panel Version 2 for a subject, age 69, with uterus cancer.

FIG. 14A is a chart showing the copy number calls across all chromosomes as determined by WGS for a subject, age 46, with triple-negative breast cancer.

FIG. 14B is a chart showing the copy number calls across all chromosomes as determined using an SNV Panel Version 2 for a subject, age 46, with triple-negative breast cancer.

FIG. 15A is a bar chart showing the frequency of biallelic ATM mutations across cancers.

FIG. 15B is a bar chart showing the frequency of biallelic germline ATM mutations across cancers.

FIG. 16 is a chart showing the sequencing coverage (x reads) observed for a series of downstream positions downstream from the primer binding site. This chart shows that better quality samples produce higher sequencing coverage over all positions. For the panel of normal samples, only good and fair samples were used.

FIG. 17A is a chart showing total copy number log ratios for a sample including cells amplified for CCNE1. The methods described herein identified the sample as having 31 total copies of CCNE1, and, after normalization to diploid, 30 copies of CCNE1.

FIG. 17B is a chart showing total copy number log ratios for a sample including cells amplified for CCNE1. The methods described herein identified the sample as having 31 total copies of CCNE1, and, after normalization to diploid, 17 copies of CCNE1.

FIG. 17C is a chart showing total copy number log ratios for a sample including cells amplified for CCNE1. The methods described herein identified the sample as having 9 total copies of CCNE1, and, after normalization to diploid, 5 copies of CCNE1.

FIGS. 18A and 18B are chart showing the total copy numbers, as determined using SNV Panel Version 2 (FIG. 18A) and WGS (FIG. 18B). In FIG. 18A, the total copy number for CCNE1 was determined to be 13 and, after normalization to diploid, 9. In FIG. 18B, the total copy number for CONE1 was determined to be 26 and, after normalization to diploid, 19.

FIGS. 19A and 19B are chart showing the total copy numbers, as determined using SNV Panel Version 2 (FIG. 19A) and WGS (FIG. 19B). In FIG. 19A, the total copy number for CCNE1 was determined to be 8 and, after normalization to diploid, 8. In FIG. 19B, the total copy number for CCNE1 was determined to be 40 and, after normalization to diploid, 19.

FIGS. 20A and 20B are chart showing the total copy numbers, as determined using SNV Panel Version 2 (FIG. 20A) and WGS (FIG. 20B). In FIG. 20A, the total copy number for CCNE1 was determined to be 10 and, after normalization to diploid, 6. In FIG. 20B, the total copy number for CCNE1 was determined to be 19 and, after normalization to diploid, 11.

FIG. 21 is a of total copy numbers as determined using SNV Panel Version 2 and WGS Ascat for various genes (N=24 samples, 605 genes), including CCNE1.

### DETAILED DESCRIPTION

In general, the invention relates to methods of treating cancers having a biallelic ATM loss of function mutation, inducing cell death in a cancer cell having a biallelic ATM loss of function mutation, or identifying a target gene mutation (e.g., ATM).

The methods described herein for treating cancer or inducing cell death typically utilize ATR inhibitors. The cancer may be, e.g., lung adenocarcinoma, adrenocortical carcinoma, breast invasive carcinoma (e.g., breast invasive carcinoma: LumB; breast invasive carcinoma: Her2; or breast invasive carcinoma: basal), pancreatic adenocarcinoma, bladder urothelial carcinoma, rectum adenocarcinoma, stomach adenocarcinoma, skin cutaneous melanoma, colon adenocarcinoma, prostate adenocarcinoma, glioblastoma multiforme, esophageal carcinoma, uterine corpus endometrial carcinoma, liver hepatocellular carcinoma, uterine corpus endometrial carcinoma, lung squamous cell carcinoma, a sarcoma, or ovarian serous cystadenocarcinoma.

Advantageously, the methods of the invention utilize ATR inhibitors to treat cancers found to be particularly responsive to ATR inhibition; these cancers have a biallelic ATM loss of function mutation.

The methods of the invention also address a problem of distinguishing a biallelic loss-of-function mutation from a monoallelic loss-of-function mutation as well as distinguishing germline and somatic mutations. Advantageously, the methods of the invention expressly account for sample purity and therefore are substantially unaffected by contaminated samples. A further advantage of the methods of the invention is in that, they can utilize pre-existing data from a panel of normal samples (normal non-cancerous tissue from a reference population) and do not require a normal tissue sample from the subject.

Typically, the subjects have a monoallelic germline (e.g., ATM) loss of function mutation and subsequently acquire a somatic loss of function mutation for the same gene (e.g., ATM). These subjects thus have a biallelic (e.g., ATM) loss of function mutation.

### Identification of Biallelic Loss of Function

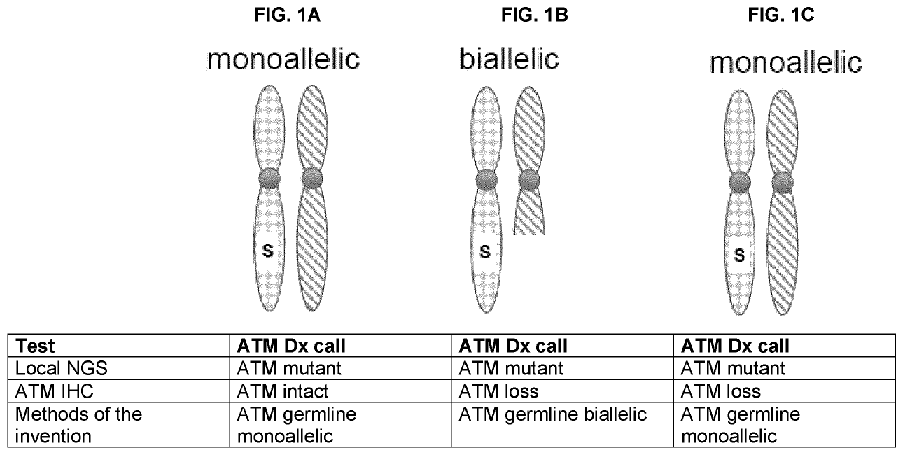

A subject or a cancer cell therefrom may be identified as having a biallelic loss of function for a gene using, e.g., Whole Genome Sequencing (WGS) or Whole Exome Sequencing (WES). Methods of the invention address the need for identification of biallelic loss of function mutation. Three exemplary mechanisms of loss of function mutations are illustrated in FIGS. 1A-1C. FIGS. 1A and 1C illustrate monoallelic loss of function mutations, and FIG. 1B illustrates a biallelic loss of function mutation. Typical next generation sequencing techniques used in cancer tests fail to distinguish between these mechanisms. Immunohistochemistry (IHC) fails to distinguish between the biallelic mutation in FIG. 1B and the monoallelic mutation in FIG. 1C, which results in an apparent ATM protein loss. As described herein, the methods involving identification of the biallelic ATM loss of function mutation distinguish the biallelic mutation in FIG. 1B from the monoallelic mutations in FIGS. 1A and 1C.

Advantageously, methods presented herein identify a subject or a cancer cell therefrom as having a biallelic loss of function for a gene but with greater cost efficiency and target gene coverage than WGS and WES techniques.

Typically, a method of the invention may include a step of determining from read counts for a plurality of single nucleotide variants (SNVs) including homozygous and heterozygous SNVs obtained from sequencing a sample including the cancer cell and from reference read counts, determining an integer total copy number of a locus segment within a target gene (e.g., ATM) region in a cancer cell from the subject or in the cancer cell and/or two integer allele-specific copy numbers of the locus segment, wherein the cancer is identified as having a biallelic (e.g., ATM) loss of function mutation if at least one of the integer total copy number and the integer allele-specific copy numbers is 0. When the integer total copy number is 0, the detected mutation is a homozygous deletion. Thus, the homozygous deletion would indicate a biallelic loss-of-function mutation for the target gene (e.g., ATM). When the integer total copy number is >0, and the integer allele-specified copy number is 0 (e.g., at the locus where the ATM-inactivating mutation is found), the detected mutation is a loss-of-heterozygosity. Thus, if the remaining target gene (e.g., ATM) allele comprises an inactivating mutation, the integer allele-specified copy number of 0 would indicate that the subject has a biallelic loss-of-function mutation for the target gene (e.g., ATM). For example, the step of determining may include: from read counts for the plurality of SNVs including homozygous and heterozygous SNVs obtained from sequencing a sample comprising the cancer cell and from reference read counts, determining total copy number log-ratios, allelic copy number log-odds ratios, and target coverage values for the heterozygous SNVs; segmenting the total copy number log-ratios and the allelic copy number log-odds ratios; estimating sample purity and sample ploidy for the cancer cell from the total copy number log-ratios and the target coverage values; and from the target coverage values, the sample purity, the sample ploidy, the total copy number log-ratios, and the allelic copy number log-odds ratios, generating an integer total copy number of a segment comprising a plurality of heterozygous single nucleotide variants (SNVs) within a target gene region (e.g., ATM gene region) in the cancer cell and two integer allele-specific copy numbers of the segment. Typically, the cell from the subject is provided as a biopsy. Read counts may be obtained using next generation sequencing of the cells in the sample.

Alternatively, the method of the invention may utilize B allele frequency analysis to identify biallelic (e.g., ATM) loss of function. For example, this method may include: determining a plurality of allele fractions for SNVs within a target gene region (e.g., ATM gene region) in a cancer cell from the subject or in the cancer cell; and segmenting the plurality of allele fractions to produce a plurality of constant allele fraction segments, wherein the cancer is identified as having a biallelic loss of function mutation (e.g., biallelic ATM loss of function mutation) if the target gene region (e.g., ATM gene region) comprises a locus of SNVs lacking segments with allele fractions between 0.05 and 0.95.

Among the methods described herein, the methods utilizing integer allele-specific copy numbers and integer total copy numbers are advantageous over others, as these methods are robust and could be used to process low purity samples. Additionally, the methods described herein and utilizing integer allele-specific copy numbers and integer total copy numbers can utilize pre-existing data from a panel of normal samples from a reference population and do not require a normal tissue sample from the subject. Thus, such a method allows for determination of a biallelic loss-of-function mutation based on a single sample (e.g., a biopsy) form the subject.

### Target SNVs

Target SNVs to be used in the methods of the invention can be selected from those known in the art according to several selection criteria identified below. The SNVs can be found, e.g., at gnomad.broadinstitute.org.

A target SNV is preferably consistently covered across samples. A target SNV is consistently covered across samples, if its mean coverage is at least 50× reads (e.g., at least 100× reads, at least 200× reads, at least 300× reads, at least 400× reads, or at least 500× read,) across the panel of normal samples. The panel of target SNVs may have a mean coverage of at least 50× (e.g., at least 100×, at least 200×, at least 300×, at least 400×, at least 500×, at least 600×, at least 700×, at least 800×, at least 900×, or at least 1000× (e.g., 100× to 2500×, 200× to 2500×, 300× to 2500×, 400× to 2500×, 500× to 2500×, 600× to 2500×, 700× to 2500×, 800× to 2500×, 900× to 2500× or 1000× to 2500×)) across the panel of normal samples. Panel of normal samples are derived from normal tissue of the reference population, where chromosomes are expected to be normal. Panel of normal samples has SNV allele fractions of 0 to 0.1 for homozygous variants, 0.4 to 0.6 for heterozygous variants, and 0.9 to 1 for absent variants. Typically, the panel of normal samples is assembled from the samples of the same tissue type as those from the subject's sample.

A target SNV may be a frequent SNV, for example, the frequent SNV may be that which has an allele frequency of greater than 33% (e.g., 33% to 66%) in humans. Here, the assessment of allele frequency in humans may be based on an SNV source, e.g., Gnomad. The inbreeding coefficient for the reference population may be between 0 and 0.2. Additionally, a target SNV may be a proximal SNV—a consistently covered SNV that is disposed within a 3′-flanking sequence relative to the frequent SNV, the 3′-flanking sequence including at total of 300 contiguous nucleobases.

A target SNV may have a 5′-flanking sequence of at least 20 contiguous nucleobases (e.g., 20-50 contiguous nucleobases, e.g., 50 contiguous nucleobases) including 25-75% GC content. Typically, the 5′-flanking sequence is unique (i.e., the sequence of 20 contiguous nucleobases is not found elsewhere within the target genome) and does not include other SNVs.

A target SNV may be a clean SNV. A clean SNV has the variant allele fraction (VAF) values within ranges 0-0.1, 0.4-0.6, and 0.9-1 in at least 95% of samples from the reference population.

Typically, target SNVs may be detected using primer-based detection techniques (e.g., next generation sequencing techniques). For a plurality of target SNVs, a plurality of primers may be designed using techniques and methods known in the art. When selecting target SNVs from a sequenced sample containing a cancer cell from a subject, those target SNVs may be selected that are disposed within the 3′-flanking sequences relative to the binding sites for the utilized plurality of primers. The 3′-flanking sequence is typically a sequence containing 300 or fewer (e.g., 200 or fewer) contiguous nucleobases in the 3′ direction relative to the binding site for the utilized primer. The number of contiguous nucleobases selected for a 3′-flanking sequence may be affected by the level of DNA damage, and length of DNA fragments in each patient sample. For example, for the mean coverage of 100× or more (e.g., 200× or more), the 3′-flanking sequence of 200 or fewer contiguous nucleobases may be used. For example, the 3′-flanking sequence of 300 bp for samples with >17% of input DNA fragments longer than 130 bp, and the 3′-flanking sequence of 200 bp otherwise. As a general matter, the 3′-flanking sequence length may be adjusted in view of the sequencing technology utilized in the sample analysis and the sample quality; the lower quality samples (i.e., samples with high degree of DNA fragmentation) typically necessitate the use of shorter 3′-flanking sequences and/or higher mean coverage levels.

Advantageously, the method described herein does not require the subject's normal tissue sample to determine whether a mutation is monoallelic or biallelic. Instead, the method described herein may utilize reference population samples. For example, reads from the panel of normal samples may be used instead of normal reads in the BAM files.

### Total Copy Number Log-Ratio

A total copy number log-ratio (Log R) may be generated from the total read count in the cancer versus reference for all target SNVs that have at least a minimum depth of coverage in the reference. Log R provides information on total copy number ratio. Sequence read count information may be first parsed from paired cancer-reference files. A normalizing constant is calculated for each cancer/reference pair to correct for total library size. Subsampling within 150-250 bp intervals may be applied to reduce hypersegmentation in SNV-dense regions of the genome. Specifically, the expected value of Log R can be expressed as

$$E [ log R ] = log \{ ( p {1}^{*} + p {2}^{⋆} ) / 2 \} + w ( \cdot ) + λ ,$$

where p1*=p1·ϕ+(1−ϕ) and p2*=p1·ϕ+(1−ϕ) are parental copy number in the tumor sample rising from a mixed normal (1,1) and aberrant (p1,p2) copy number genotype with mixing proportion ϕ. ϕ is the cellular fraction associated with the aberrant genotype, which is a function of tumor purity and clonal frequency (for subclonal alterations). The term w(⋅) denotes systematic bias. GC-content may be explicitly considered, and loess regression of log R over GC in 1 kb windows along the genome may be used to estimate the GC-effect on read counts and subtract it from log R. In addition, Log R quantifies relative copy number, hence a constant A is included for absolute copy number conversion.

For Log R generation, sequence read count information may be first parsed form paired cancer-control BAM files. A normalizing constant may be calculated for each cancer/control pair to correct for total library size. Subsampling within 150-250 bp intervals may be applied to reduce hypersegmentation in SNV-dense regions of the genome.

### Allelic Copy Number Log-Odds Ratio

Allelic copy number log-odds ratio (log OR) of the variant-allele count in cancer versus reference allele, which is an unbiased estimate of allelic copy ratio: E[log OR]=[p1·ϕ+(1−ϕ)]/[p2·ϕ+ (1−ϕ)]), where E[log OR] is the expected value of log OR, p1 is a parental copy number of the variant allele, p2 is a parental copy number of the allele from the other parent, and ϕ is a cellular fraction that is a function of tumor purity and clonal frequency (for subclonal alterations). In the absence of phased data, squared log OR may be used to infer log² ([p1·ϕ+ (1−ϕ)]/[p2·ϕ+(1−ϕ)]).

### Joint Segmentation

Segmentation analysis may be used to identify regions of the genome that have constant copy number using change point detection methods. Conventional methods (e.g., BIC-seq, ExomeCNV) typically perform one-dimensional segmentation using log R alone, or separate application of one-dimensional segmentation to log R and B Allele Frequency (BAF).

Preferably, a circular binary segmentation (CBS) algorithm is used for a joint segmentation of log R and log OR based on a bivariate Hotelling T² statistic:

$${T}^{2} = {max}_{1 \leq i ≦ j ≦ n} {T}_{1 ij}^{2} + c {T}_{2 ij}^{2}$$

where T₁ᵢⱼ the Mann-Whitney statistic comparing the set of observed log R denoted as {X₁ₖ: i<k≤j} and its complement {X₁ₖ: 1<k≤iorj<k≤n}, and T₂ᵢⱼ is the Mann-Whitney statistic comparing the set of observed log OR denoted as {X₂ₖ: i<k≤j} and its complement {X₂ₖ: 1<k≤iorj<k≤n}. In the above, c is a scaling factor that is inversely proportional to the heterozygous rate.

Here, if the maximal statistic is greater than a pre-determined critical value, a change is declared and the change-points are estimated as i, j that maximize the statistic. This approach iteratively searches for change points between any possible pair of breakpoints and its complement to identify regions of the genome that have constant allele-specific copy number. For each segment, the log R data are summarized using the median of the log R values [symbol] and the log OR data are summarized by \tilde{x}₂² which takes the form Σ{x₂²−s²)/s²}/{1/s²}, where s² is the estimated variance of log OR.

While log R is defined for all SNVs (both homozygous and heterozygous loci), log OR is only defined for heterozygous loci (het-loci or het-SNVs). This might create an imbalance between the two in the combined statistic. To address this issue, a weight that is inversely proportional to the heterozygous rate is introduced to increase the het-SNV contributions in subsequent segmentation analysis. Specifically, a scaling factor c is introduced in the T² statistic. This is empirically set at 1/\sqrt{4γ}, where γ is the proportion of het-SNVs in the cancer cell sample. Up-weighing the contribution of log OR for het-SNVs increases the power of detecting allelic imbalances for regions with low frequency of het-SNVs.

Segmentation may be alternatively performed using, for example, a running mean method. Alternatively, the Log R and Log OR data may be divided into predetermined short segments (based on the SNV loci), and

After segmentation, the segments are clustered into groups of the same underlying genotype. Such clustering reduces the number of latent copy number and cellular fraction states needed in subsequent modeling.

### Location Shift Determination

Log R estimates are proportional to the absolute total copy number up to a location constant λ. For diploid genome, log R=0 (library size normalized log R) is the location for the 2-copy state. However, aneuploidy can lead to a location shift in the tumor. Therefore, the 2-copy state should be determined in a tumor genome, and the location constant λ should be quantified.

The copy number states may be denoted using total and minor integer copy number (e.g. 1-0 denotes monosomy with total copy number 1 and minor copy number 0). The estimate of λ should correspond to the log R level at which the segments are in 2-1 (normal diploid) or 2-0 (copy-neutral LOH) state. In order to estimate λ, normal diploid segments should be allelically balanced. Thus, candidate value for λ (referred to as λ_{c}) will be obtained from [symbol] for segment clusters that have [symbol] values close to zero.

However, homozygous deletions (0-0) and balanced gains (4-2, 6-3 etc.) are also allelically balanced and hence will have small [symbol] . Since large scale homozygous deletions of multiple genes will not be conducive to cell survival, non-focal segments with small [symbol] may be eliminated as being homozygous deletions. In addition, for the sake of simplicity, higher order balanced gains states (6-3, 8-4 etc.) spanning a large part of the genome are not considered. Samples in which segments with allelic balance are a small fraction of targeted regions are flagged and may be subjected to a manual review for their A estimates.

In samples that have large allelically balanced segments, there can be several [symbol] values from which λc can be chosen.

### Integer Copy Numbers

An integer allele-specific copy numbers (major and minor) and the associated cellular fraction estimates for each segment cluster by modeling the expected values of log R and log OR given total (t), and each parental (p1,p2) copy as a function of a cf parameter ϕ, using a combination of parametric and non-parametric methods. This allows for modeling both clonal and subclonal events.

First a moment estimate of [symbol] , the total copy number for segment cluster i, is obtained by |2^{(1-x̅}^{₁ᵢ₎}|, where [symbol] , denote the median log R for segment cluster i corrected for sequence bias and tumor ploidy (λ-normalized). Once the total number is obtained, we calculate the allele specific copy numbers m and p and the cellular fraction ϕ using the fact that the log OR summary measure [symbol] is a moment estimate of μ² which equals log²({p1·ϕ+(1−ϕ)}/{p2·ϕ+(1−ϕ)}).

To further refine the initial estimates, a Gaussian-non-central χ² model may employed with error terms to account for the noise with a clonal structure imposed on the cellular fraction ϕ. Specifically, let X₁ᵢⱼ denote the log R for SNV loci j in segment cluster i (corrected for sequence bias and location shift) and follow a normal distribution:

$${X}_{1 ij} ∼ N ( {v}_{ig} , {τ}_{i}^{2} ) ,$$

where ν_{ig} is the expected value of log R given the underlying copy number state g taking the form

$${v}_{ig} = {log}_{2} ( 2 ( 1 - {ϕ}_{k} ) + {t}_{g} {ϕ}_{k} ) / 2 ,$$

where t_{g}=p1_{g}+p2_{g} denotes the total copy number (sum of the two parental copy number) given the underlying copy number state g, ϕₖ denotes the cellular fraction for clonal cluster k, and τᵢ² is an independent variance parameter. In practice, it is reasonable to assume homoscedasticity and set τᵢ²=τ²∀i.

Furthermore, let X₂ᵢⱼ denote the log OR for SNV loci j in segment cluster i and (X₂ᵢⱼ/σᵢⱼ)² follow a non-central chi-squared distribution:

$${( {X}_{2 ij} / {σ}_{ij} )}^{2} ∼ {χ}^{2} ( {δ}_{ijg} ) ,$$

where σᵢⱼ² is the variance parameter for log OR and δ_{ijg}=μ_{ig}²/σᵢⱼ² is the non-centrality parameter in which

$${μ}_{ig}^{2} = {log}_{2} ( ( {m}_{g} {ϕ}_{k} + ( 1 - {ϕ}_{k} ) ) / ( {p}_{g} {ϕ}_{k} + ( 1 - {ϕ}_{k} ) ) ) .$$

Assuming X₁ᵢⱼ and X₂ᵢⱼ are independent random variables given the underlying copy number state g, the joint data likelihood can then be written as

$$ℓ = {\sum }_{i} {\sum }_{j} {\sum }_{g} f ( {x}_{1 ij} \vert {v}_{ig} , {τ}_{i}^{2} , g ) f ( {x}_{2 ij} \vert {δ}_{ijg} , g ) P ( g )$$

where P(g) is the prior probability of the latent copy number state g.

An expectation-maximization (EM) algorithm may be applied to improve the joint data likelihood. It can be viewed as an estimation problem with the latent copy number states as missing data. In the E-step of the EM procedure, Bayes theorem is used to compute the posterior probability of segment cluster i being assigned copy number state g given the parameter estimates at the tth iteration:

$${\hat{p}}_{ijg}^{( t )} = \frac{f ( {x}_{1 i j} \vert {\hat{v}}_{i g}^{( t )} , {\hat{τ}}_{i}^{2 ( t )} , g ) f ( {x}_{2 i j} \vert {\hat{δ}}_{ijg}^{( t )} ) P ( g )}{{\sum }_{g} f ( {x}_{1 ij} \vert {\hat{v}}_{ig} , {\hat{τ}}_{i}^{2 ( t )} , g ) f ( {x}_{2 ij} \vert {\hat{δ}}_{ijg}^{( t )} ) P ( g )} .$$

In the M-step, we first update the normal and non-central Chi-square distribution parameters

$${\hat{v}}_{ig}^{( i + 1 )} = \frac{{\sum }_{j} {\hat{p}}_{ijg}^{( t )} \cdot {x}_{1 i j}}{{\sum }_{j} {\hat{p}}_{ijg}^{( t )}} ,$$

$${τ}_{i}^{2 ( i )} = \frac{{\sum }_{j} {{\hat{p}}_{ijg}^{( t )} ( {x}_{1 i j} - {\hat{v}}_{ig}^{( t )} )}^{2}}{{\sum }_{j} {\hat{p}}_{ijg}^{( t )}} ,$$

$${\hat{μ}}_{ig}^{2 ( i + 1 )} = \frac{{\sum }_{j} {\hat{p}}_{ijg}^{( t )} \cdot ( {x}_{2 ij}^{2} - {s}^{2} ) / {s}^{2}}{{\sum }_{j} {\hat{p}}_{ijg}^{( t )} / {s}^{2}} ,$$

where s² is the sample variance estimate of log OR. After obtaining the estimates of v and then update the cellular fraction parameter ϕₖ^{(t+1)} given

$${v}_{{ig}^{*}}^{( t - 1 )} = log \frac{2 ( 1 - {ϕ}_{k} ) + {t}_{g} \text{?} {ϕ}_{k}}{2} ,$$

$${\hat{μ}}_{{ig}^{*}}^{( t + 1 )} = log \frac{{m}_{g} \text{?} {ϕ}_{i} + ( 1 - {ϕ}_{k} )}{{n}_{g} \text{?} {ϕ}_{k} + ( 1 - {ϕ}_{k} )} ,$$

$$\text{?} \text{indicates text missing or illegible when filed}$$

where g* is the most likely genotype (with highest posterior probability) given the data and current parameter estimates in the tth iteration. The E-step and M-step are iterated until convergence.

A clonal structure is imposed on the cellular fraction ϕₖ. This is done in a sequential approach where the algorithm starts with a single clonal cluster (k=1) with cellular fraction parameter ϕ₁. Then, the method may involve identification of segment clusters for which segment cluster-specific estimates are non-trivially lower (at least by 0.05) from the clonally constrained estimates that result in a suboptimal fit under k=1. These segment clusters with discordant cellular fraction estimates then form a candidate subclonal cluster of events at a lower cellular fraction ϕ₂, and a model is fitted with the joint likelihood optimized under k=2. This procedure is iterated until no additional discordance in cellular fraction estimates are found, or a specified maximum k (e.g., k=5) is reached, as desired and depending on the intratumor heterogeneity. In the output, [symbol] is the cellular fraction estimate for the clonal events and also the tumor purity by definition, and [symbol] , k>1 for any subclonal clusters identified in the sample.

### Distinguishing Germline and Somatic Mutations

The methods described herein may be used to identify a target mutation as germline or somatic.

Using methods described herein, identification of the target mutation as a germline or somatic mutation may be achieved with or without the use of a normal, matched sample from the subject (in addition to the sample containing a cancer cell from the subject, e.g., a biopsy). A normal, matched sample from the same subject is a sample containing normal (non-cancerous) cells, e.g., a blood sample from the subject.

In instances where a sample containing a cancer cell from the subject (e.g., a biopsy from the subject) and a normal, matched sample from the subject (e.g., a blood sample from the subject) are available, the methods described herein may include the step of identifying a mutation in the normal, matched sample from the subject. If the target mutation present in the cancer cell from the subject is identified in the normal, matched sample, the target mutation is germline. If the target mutation present in the cancer cell from the subject is not identified in the normal, matched sample, the target mutation is somatic.

For example, in instances where a normal, matched sample from the subject is unavailable, the methods described herein may include the steps of:
from read counts for a plurality of consistently covered single nucleotide variants (SNVs) comprising homozygous and heterozygous consistently covered SNVs obtained from sequencing a sample comprising the cell, determining an observed allele fraction of the target mutation, determining expected allele fractions (E[a]) for a germline mutation and for a somatic mutation, where the allele fraction for the germline mutation is E[a]=[(1−ϕ)·1+ϕ·m_{cn}]/[(1−ϕ)·2+ϕ·tₜᵤᵣₙ], and m_{cn} is p1 or p2, tₜᵤᵣₙ is total copy number of the locus in tumor, and where the allele fractions for the somatic mutation are E[a]=[ϕ·m_{cn}]/[(1−ϕ)·2+ϕ·tₜᵤᵣₙ] enumerated for all m_{cn}≤min(p1,p2), where m_{cn} is a mutant allele copy number for the genome region including the target mutation, ϕ is a cellular fraction (used as a measure of the sample purity), p1 is a parental copy number of the variant allele, p2 is a parental copy number of the allele from the other parent,
comparing the observed allele fraction to the expected allele fractions to identify the most probable of the germline and somatic mutations, and
identifying the target mutation as germline or somatic as that which is the most probable of the germline and somatic mutations.

The mutant allele copy number (m_{cn}) is an integer from 1 to tₜᵤᵣₙ, where tₜᵤᵣₙ is the total copy number of alleles for the region of interest in the cancer cell from the subject. The comparing step may be performed using Bayesian model comparison (Bayes factor).

This approach presumes that the normal cells are diploid and that the sample from the subject is impure (ϕ<1). As ϕ approaches 1, the germline and somatic mutations become indistinguishable in this approach in the absence of the normal, matched sample from the subject.

Alternatively, e.g., in instances where a normal, matched sample from the subject is unavailable, and the sample containing a cancer cell from the subject is impure (ϕ<0.9, or 90%), the methods described herein may be used to identify a target mutation as somatic if the observed allele fraction is outside the same as the expected allele fraction (unadjusted for purity). For example, for a total copy number of 2, SNVs would be expected to occur within an allele fraction range (unadjusted for purity) of less than 10% (homozygous SNV that is absent), 40-60% (e.g., 45-55%) (heterozygous SNV), and greater than 90% (homozygous SNV that is present); therefore, the target mutation is somatic, if its observed allele fraction is outside the expected allele fraction ranges. If the observed allele fraction is within an expected allele fraction range, this particular approach does not permit characterizing the target mutation.

### Identification of Target Gene Overexpression

A subject or a cancer cell therefrom may be identified as amplified for a target gene using the methods as described for a biallelic loss of function mutation identification with the exception that the cell is identified as amplified for a target gene, if the total copy number is at least double the sample ploidy, or if the total copy number is greater than the sample ploidy by at least two units.

The total copy number here is defined as,

$$t = p 1 + p 2 , or$$

$$t = [ {2}^{( log R - λ + 1 )} - 2 + 2 ϕ ] / ϕ ,$$

where p1*=p1·ϕ+(1−ϕ) and p2*=p1·ϕ+ (1−ϕ) are parental copy number in the tumor sample rising from a mixed normal (1,1) and aberrant (p1,p2) copy number genotype with mixing proportion ϕ. The total copy number thus defined is an unnormalized copy number. ϕ is the cellular fraction associated with the aberrant genotype, which is a function of tumor purity and clonal frequency (for subclonal alterations). The term w(⋅) denotes systematic bias. GC-content may be explicitly considered, and loess regression of log R over GC in 1 kb windows along the genome may be used to estimate the GC-effect on read counts and subtract it from log R. In addition, Log R quantifies relative copy number, hence a constant λ is included for absolute copy number conversion.

For Log R generation, sequence read count information may be first parsed form paired cancer-control BAM files. A normalizing constant may be calculated for each cancer/control pair to correct for total library size. Subsampling within 150-250 bp intervals may be applied to reduce hypersegmentation in SNV-dense regions of the genome.

Thus, the invention provides a method of identifying a cell from a subject as amplified for a target gene, the method including the step of:
from read counts for a plurality of consistently covered single nucleotide variants (SNVs) including homozygous and heterozygous consistently covered SNVs obtained from sequencing a sample including the cell and from reference read counts, determining a total copy number of a locus segment within a target gene region in the cell from the subject and a sample ploidy, the target gene region including the mutation, where the reference read counts are from a panel of normal samples,
where the cell is identified as amplified for target gene, if the total copy number is at least double (e.g., at least double) the sample ploidy, or if the total copy number is greater than the sample ploidy by at least two (e.g., at least three).

In some embodiments, the determining step includes:
from the read counts and the reference read counts, determining total copy number log-ratios, and target coverage values for the SNVs;
segmenting the total copy number log-ratios;
estimating sample purity and the sample ploidy for the cell from the total copy number log-ratios and the target coverage values; and
from the target coverage values, the sample purity, the sample ploidy, and the total copy number log-ratios, generating the total copy number of a locus segment within a target gene region.

The total copy number may be normalized to ploidy as follows: tₙ=2·t/p2, where tₙ is a normalized total copy number, t is an unnormalized copy number, and p2 is as defined above.

### Biallelic ATM Loss of Function Mutation Identification

Methods described herein thus may include a step of identifying the cancer as having a biallelic ATM loss of function mutation using the techniques described above.

### Identification of CCNE1 Overexpression

Methods described herein thus may include a step of identifying the cancer as amplified for CCNE1 using the techniques described above.

### SNV Detection

Detection techniques for evaluating nucleic acids for the presence of a SNV involve procedures well known in the field of molecular genetics. Many, but not all, of the methods involve amplification of nucleic acids. Ample guidance for performing amplification is provided in the art. Exemplary references include manuals such as PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Current Protocols in Molecular Biology, Ausubel, 1994-1999, including supplemental updates through April 2004; Sambrook & Russell, Molecular Cloning, A Laboratory Manual (3rd Ed, 2001). General methods for detection of single nucleotide variants are disclosed in Single Nucleotide Variants: Methods and Protocols, Pui-Yan Kwok, ed., 2003, Humana Press. SNV detection methods often employ labeled oligonucleotides. Oligonucleotides can be labeled by incorporating a label detectable by spectroscopic, photochemical, biochemical, immunochemical, or chemical means. Useful labels include fluorescent dyes, radioactive labels, e.g. 32P, electron-dense reagents, enzyme, such as peroxidase or alkaline phosphatase, biotin, or haptens and proteins for which antisera or monoclonal antibodies are available. Labeling techniques are well known in the art (see, e.g. Current Protocols in Molecular Biology, supra; Sambrook & Russell, supra).

Although the methods typically employ PCR steps, other amplification protocols may also be used. Suitable amplification methods include ligase chain reaction (see, e.g., Wu & Wallace, Genomics 4:560-569, 1988); strand displacement assay (see, e.g. Walker et al., Proc. Natl. Acad. Sci. USA 89:392-396, 1992; U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al., Proc. Natl. Acad. Sci. USA 86:1173-1177, 1989); and self-sustained sequence replication (3SR) (Guatelli et al., Proc. Natl. Acad. Sci. USA 87:1874-1878, 1990; WO 92/08800). Alternatively, methods that amplify the probe to detectable levels can be used, such as Qβ-replicase amplification (Kramer & Lizardi, Nature 339:401-402, 1989; Lomeli et al., Clin. Chem. 35:1826-1831, 1989). A review of known amplification methods is provided, for example, by Abramson and Myers in Curr. Op Biotechnol. 4:41-47, 1993.

Detection of the genotype, haplotype, SNV, microsatellite, or other variant of an individual can be performed using oligonucleotide primers and/or probes. Oligonucleotides can be prepared by any suitable method, usually chemical synthesis. Oligonucleotides can be synthesized using commercially available reagents and instruments. Alternatively, they can be purchased through commercial sources. Methods of synthesizing oligonucleotides are well known in the art (see, e.g., Narang et al., Meth. Enzymol. 68:90-99, 1979; Brown et al., Meth. Enzymol. 68:109-151, 1979; Beaucage et al., Tetrahedron Lett. 22:1859-1862, 1981; and the solid support method of U.S. Pat. No. 4,458,066). In addition, modifications to the above-described methods of synthesis may be used to desirably impact enzyme behavior with respect to the synthesized oligonucleotides. For example, incorporation of modified phosphodiester linkages (e.g., phosphorothioate, methylphosphonates, phosphoamidate, or boranophosphate) or linkages other than a phosphorous acid derivative into an oligonucleotide may be used to prevent cleavage at a selected site. In addition, the use of 2′-amino modified sugars tends to favor displacement over digestion of the oligonucleotide when hybridized to a nucleic acid that is also the template for synthesis of a new nucleic acid strand.

The genotype of an individual can be determined using many detection methods that are well known in the art. Most assays entail one of several general protocols: hybridization using allele-specific oligonucleotides, primer extension, allele-specific ligation, sequencing, or electrophoretic separation techniques, e.g., single-stranded conformational variant (SSCP) and heteroduplex analysis. Exemplary assays include 5′-nuclease assays, template-directed dye-terminator incorporation, molecular beacon allele-specific oligonucleotide assays, single-base extension assays, and SNV scoring by real-time pyrophosphate sequences. Analysis of amplified sequences can be performed using various technologies such as microchips, fluorescence polarization assays, and MALDI-TOF (matrix assisted laser desorption ionization-time of flight) mass spectrometry. Two methods that can also be used are assays based on invasive cleavage with Flap nucleases and methodologies employing padlock probes.

Determination of the presence or absence of a particular allele is generally performed by analyzing a nucleic acid sample that is obtained from the individual to be analyzed. Often, the nucleic acid sample comprises genomic DNA. The genomic DNA is typically obtained from blood samples but may also be obtained from other cells or tissues.

It is also possible to analyze RNA samples for the presence of polymorphic alleles. For example, mRNA can be used to determine the genotype of an individual at one or more polymorphic sites. In this case, the nucleic acid sample is obtained from cells in which the target nucleic acid is expressed, e.g., adipocytes. Such an analysis can be performed by first reverse-transcribing the target RNA using, e.g., a viral reverse transcriptase, and then amplifying the resulting cDNA; or using a combined high-temperature reverse-transcription-polymerase chain reaction (RT-PCR), as described in U.S. Pat. Nos. 5,310,652; 5,322,770; 5,561,058; 5,641,864; and 5,693,517.

Frequently used methodologies for analysis of nucleic acid samples to detect SNVs are briefly described. However, any method known in the art can be used in the invention to detect the presence of single nucleotide substitutions.

### Allele-Specific Hybridization

Allele-specific hybridization, also commonly referred to as allele specific oligonucleotide hybridization (ASO) (e.g., Stoneking et al., Am. J. Hum. Genet. 48:70-382, 1991; Saiki et al., Nature 324, 163-166, 1986; EP 235,726; and WO 89/11548), relies on distinguishing between two DNA molecules differing by one base by hybridizing an oligonucleotide probe that is specific for one of the variants to an amplified product obtained from amplifying the nucleic acid sample. This method typically employs short oligonucleotides, e.g. 15-20 bases in length. The probes are designed to differentially hybridize to one variant versus another. Principles and guidance for designing such probe is available in the art, e.g. in the references cited herein. Hybridization conditions should be sufficiently stringent that there is a significant difference in hybridization intensity between alleles, and producing an essentially binary response, whereby a probe hybridizes to only one of the alleles. Some probes are designed to hybridize to a segment of target DNA such that the polymorphic site aligns with a central position (e.g., in a 15-base oligonucleotide at the 7 position; in a 16-based oligonucleotide at either the 8 or 9 position) of the probe, but this design is not required.

The amount and/or presence of an allele is determined by measuring the amount of allele-specific oligonucleotide that is hybridized to the sample. Typically, the oligonucleotide is labeled with a label such as a fluorescent label. For example, an allele-specific oligonucleotide is applied to immobilized oligonucleotides representing SNV sequences. After stringent hybridization and washing conditions, fluorescence intensity is measured for each SNV oligonucleotide.

In one embodiment, the nucleotide present at the polymorphic site is identified by hybridization under sequence-specific hybridization conditions with an oligonucleotide probe or primer exactly complementary to one of the polymorphic alleles in a region encompassing the polymorphic site. The probe or primer hybridizing sequence and sequence-specific hybridization conditions are selected such that a single mismatch at the polymorphic site destabilizes the hybridization duplex sufficiently so that it is effectively not formed. Thus, under sequence-specific hybridization conditions, stable duplexes will form only between the probe or primer and the exactly complementary allelic sequence. Thus, oligonucleotides from about 10 to about 35 nucleotides in length, usually from about 15 to about 35 nucleotides in length, which are exactly complementary to an allele sequence in a region which encompasses the polymorphic site are within the scope of the invention.

In an alternative embodiment, the nucleotide present at the polymorphic site is identified by hybridization under sufficiently stringent hybridization conditions with an oligonucleotide substantially complementary to one of the SNV alleles in a region encompassing the polymorphic site, and exactly complementary to the allele at the polymorphic site. Because mismatches which occur at non-polymorphic sites are mismatches with both allele sequences, the difference in the number of mismatches in a duplex formed with the target allele sequence and in a duplex formed with the corresponding non-target allele sequence is the same as when an oligonucleotide exactly complementary to the target allele sequence is used. In this embodiment, the hybridization conditions are relaxed sufficiently to allow the formation of stable duplexes with the target sequence, while maintaining sufficient stringency to preclude the formation of stable duplexes with non-target sequences. Under such sufficiently stringent hybridization conditions, stable duplexes will form only between the probe or primer and the target allele. Thus, oligonucleotides from about 10 to about 35 nucleotides in length, usually from about 15 to about 35 nucleotides in length, which are substantially complementary to an allele sequence in a region which encompasses the polymorphic site and are exactly complementary to the allele sequence at the polymorphic site, are within the scope of the invention.

The use of substantially, rather than exactly, complementary oligonucleotides may be desirable in assay formats in which optimization of hybridization conditions is limited. For example, in a typical multi-target immobilized-oligonucleotide assay format, probes or primers for each target are immobilized on a single solid support. Hybridizations are carried out simultaneously by contacting the solid support with a solution containing target DNA. As all hybridizations are carried out under identical conditions, the hybridization conditions cannot be separately optimized for each probe or primer. The incorporation of mismatches into a probe or primer can be used to adjust duplex stability when the assay format precludes adjusting the hybridization conditions. The effect of a particular introduced mismatch on duplex stability is well known, and the duplex stability can be routinely both estimated and empirically determined, as described above. Suitable hybridization conditions, which depend on the exact size and sequence of the probe or primer, can be selected empirically using the guidance provided herein and well known in the art. The use of oligonucleotide probes or primers to detect single base pair differences in sequence is described in, e.g., Conner et al., Proc. Natl. Acad. Sci. USA 80:278-282, 1983, and U.S. Pat. Nos. 5,468,613 and 5,604,099, each incorporated herein by reference.

The proportional change in stability between a perfectly matched and a single-base mismatched hybridization duplex depends on the length of the hybridized oligonucleotides. Duplexes formed with shorter probe sequences are destabilized proportionally more by the presence of a mismatch. Oligonucleotides between about 15 and about 35 nucleotides in length are often used for sequence-specific detection. Furthermore, because the ends of a hybridized oligonucleotide undergo continuous random dissociation and re-annealing due to thermal energy, a mismatch at either end destabilizes the hybridization duplex less than a mismatch occurring internally. For discrimination of a single base pair change in target sequence, the probe sequence that hybridizes to the target sequence is selected such that the polymorphic site occurs in the interior region of the probe.

The above criteria for selecting a probe sequence that hybridizes to a specific allele apply to the hybridizing region of the probe, i.e., that part of the probe which is involved in hybridization with the target sequence. A probe may be bound to an additional nucleic acid sequence, such as a poly-T tail used to immobilize the probe, without significantly altering the hybridization characteristics of the probe. One of skill in the art will recognize that for use in the present methods, a probe bound to an additional nucleic acid sequence which is not complementary to the target sequence and, thus, is not involved in the hybridization, is essentially equivalent to the unbound probe.

Suitable assay formats for detecting hybrids formed between probes and target nucleic acid sequences in a sample are known in the art and include the immobilized target (dot-blot) format and immobilized probe (reverse dot-blot or line-blot) assay formats. Dot blot and reverse dot blot assay formats are described in U.S. Pat. Nos. 5,310,893; 5,451,512; 5,468,613; and 5,604,099; each incorporated herein by reference.

In a dot-blot format, amplified target DNA is immobilized on a solid support, such as a nylon membrane. The membrane-target complex is incubated with labeled probe under suitable hybridization conditions, unhybridized probe is removed by washing under suitably stringent conditions, and the membrane is monitored for the presence of bound probe.

In the reverse dot-blot (or line-blot) format, the probes are immobilized on a solid support, such as a nylon membrane or a microtiter plate. The target DNA is labeled, typically during amplification by the incorporation of labeled primers. One or both of the primers can be labeled. The membrane-probe complex is incubated with the labeled amplified target DNA under suitable hybridization conditions, unhybridized target DNA is removed by washing under suitably stringent conditions, and the membrane is monitored for the presence of bound target DNA. A reverse line-blot detection assay is described in the example.

An allele-specific probe that is specific for one of the variant variants is often used in conjunction with the allele-specific probe for the other variant variant. In some embodiments, the probes are immobilized on a solid support and the target sequence in an individual is analyzed using both probes simultaneously. Examples of nucleic acid arrays are described by WO 95/11995. The same array or a different array can be used for analysis of characterized variants. WO 95/11995 also describes subarrays that are optimized for detection of variant forms of a pre-characterized variant. Such a subarray can be used in detecting the presence of the variants described herein.

### Allele-Specific Primers

Variants are also commonly detected using allele-specific amplification or primer extension methods. These reactions typically involve use of primers that are designed to specifically target a variant via a mismatch at the 3′-end of a primer. The presence of a mismatch effects the ability of a polymerase to extend a primer when the polymerase lacks error-correcting activity. For example, to detect an allele sequence using an allele-specific amplification- or extension-based method, a primer complementary to one allele of a variant is designed such that the 3′-terminal nucleotide hybridizes at the polymorphic position. The presence of the particular allele can be determined by the ability of the primer to initiate extension. If the 3′-terminus is mismatched, the extension is impeded.

In some embodiments, the primer is used in conjunction with a second primer in an amplification reaction. The second primer hybridizes at a site unrelated to the polymorphic position. Amplification proceeds from the two primers leading to a detectable product signifying the particular allelic form is present. Allele-specific amplification- or extension-based methods are described in, e.g., WO 93/22456; U.S. Pat. Nos. 5,137,806; 5,595,890; 5,639,611; and 4,851,331.

Using allele-specific amplification-based genotyping, identification of the alleles requires only detection of the presence or absence of amplified target sequences. Methods for the detection of amplified target sequences are well known in the art. For example, gel electrophoresis and probe hybridization assays described are often used to detect the presence of nucleic acids.

In an alternative probe-less method, the amplified nucleic acid is detected by monitoring the increase in the total amount of double-stranded DNA in the reaction mixture, is described, e.g. in U.S. Pat. No. 5,994,056; and European Patent Publication Nos. 487,218 and 512,334. The detection of double-stranded target DNA relies on the increased fluorescence various DNA-binding dyes, e.g., SYBR Green, exhibit when bound to double-stranded DNA.

As appreciated by one in the art, allele-specific amplification methods can be performed in reaction that employ multiple allele-specific primers to target particular alleles. Primers for such multiplex applications are generally labeled with distinguishable labels or are selected such that the amplification products produced from the alleles are distinguishable by size. Thus, for example, both alleles in a single sample can be identified using a single amplification by gel analysis of the amplification product.

As in the case of allele-specific probes, an allele-specific oligonucleotide primer may be exactly complementary to one of the polymorphic alleles in the hybridizing region or may have some mismatches at positions other than the 3′-terminus of the oligonucleotide, which mismatches occur at non-polymorphic sites in both allele sequences.

### Detectable Probes

### 5′-Nuclease Assay Probes

Genotyping can also be performed using a “TaqMan®” or “5′-nuclease assay”, e.g., as described in U.S. Pat. Nos. 5,210,015; 5,487,972; and 5,804,375; and Holland et al., Proc. Natl. Acad. Sci. USA 88:7276-72801988. In the TaqMan® assay, labeled detection probes that hybridize within the amplified region are added during the amplification reaction. The probes are modified so as to prevent the probes from acting as primers for DNA synthesis. The amplification is performed using a DNA polymerase having 5′- to 3′-exonuclease activity. During each synthesis step of the amplification, any probe which hybridizes to the target nucleic acid downstream from the primer being extended is degraded by the 5′- to 3′-exonuclease activity of the DNA polymerase. Thus, the synthesis of a new target strand also results in the degradation of a probe, and the accumulation of degradation product provides a measure of the synthesis of target sequences.

The hybridization probe can be an allele-specific probe that discriminates between the SNV alleles. Alternatively, the method can be performed using an allele-specific primer and a labeled probe that binds to amplified product.

Any method suitable for detecting degradation product can be used in a 5′-nuclease assay. Often, the detection probe is labeled with two fluorescent dyes, one of which is capable of quenching the fluorescence of the other dye. The dyes are attached to the probe, usually one attached to the 5′-terminus and the other is attached to an internal site, such that quenching occurs when the probe is in an unhybridized state and such that cleavage of the probe by the 5′- to 3′-exonuclease activity of the DNA polymerase occurs in between the two dyes. Amplification results in cleavage of the probe between the dyes with a concomitant elimination of quenching and an increase in the fluorescence observable from the initially quenched dye. The accumulation of degradation product is monitored by measuring the increase in reaction fluorescence. U.S. Pat. Nos. 5,491,063 and 5,571,673, both incorporated herein by reference, describe alternative methods for detecting the degradation of probe which occurs concomitant with amplification.

### Secondary Structure Probes

Probes detectable upon a secondary structural change are also suitable for detection of a variant, including SNVs. Exemplified secondary structure or stem-loop structure probes include molecular beacons or Scorpion® primer/probes. Molecular beacon probes are single-stranded oligonucleic acid probes that can form a hairpin structure in which a fluorophore and a quencher are usually placed on the opposite ends of the oligonucleotide. At either end of the probe short complementary sequences allow for the formation of an intramolecular stem, which enables the fluorophore and the quencher to come into close proximity. The loop portion of the molecular beacon is complementary to a target nucleic acid of interest. Binding of this probe to its target nucleic acid of interest forms a hybrid that forces the stem apart. This causes a conformation change that moves the fluorophore and the quencher away from each other and leads to a more intense fluorescent signal. Molecular beacon probes are, however, highly sensitive to small sequence variation in the probe target (Tyagi and Kramer, Nat. Biotechnol. Vol. 14, pages 303-308, 1996; Tyagi et al., Nat. Biotech, Vol. 16, pages 49-53, 1998; Piatek et al., Nat Biotechnol, 16:359-363 (1998); Marras et al., Genetic Analysis: Biomolecular Engineering, Vol14, pages 151-156 (1999); Täpp I. et al, BioTechniques. Vol 28, pages 732-738, 2000). A Scorpion® primer/probe comprises a stem-loop structure probe covalently linked to a primer.

### Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution (see, e.g., Erlich, ed., PCR Technology: Principles and Applications for DNA Amplification, W. H. Freeman and Co, New York, 1992, Chapter 7).

Distinguishing of microsatellite variants can be done using capillary electrophoresis. Capillary electrophoresis conveniently allows identification of the number of repeats in a particular microsatellite allele. The application of capillary electrophoresis to the analysis of DNA variants is well known to those in the art (see, e.g., Szantai, et al, J Chromatogr A. 1079 (1-2): 41-49, 2005; Bjørheim and Ekstrøm, Electrophoresis 26 (13): 2520-2530, 2005 and Mitchelson, Mol Biotechnol. 24 (1): 41 68, 2003).

### Single-Strand Conformation Polymorphism Analysis

Alleles of target sequences can be differentiated using single-strand conformation polymorphism analysis, which identifies base differences by alteration in electrophoretic migration of single stranded PCR products, as described, e.g., in Orita et al., Proc. Natl. Acad. Sci. USA 86 (8), 2766-2770, 1989. Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence difference between alleles of target.

### DNA Sequencing and Single Base Extensions

SNVs can also be detected by direct sequencing. Methods include e.g. dideoxy sequencing-based methods and other methods such as Maxam and Gilbert sequence (see, e.g. Sambrook and Russell, supra).

Other detection methods include Pyrosequencing™ of oligonucleotide-length products. Such methods often employ amplification techniques such as PCR. For example, in pyrosequencing, a sequencing primer is hybridized to a single stranded, PCR-amplified, DNA template; and incubated with the enzymes, DNA polymerase, ATP sulfurylase, luciferase and apyrase, and the substrates, adenosine 5′ phosphosulfate (APS) and luciferin. The first of four deoxynucleotide triphosphates (dNTP) is added to the reaction. DNA polymerase catalyzes the incorporation of the deoxynucleotide triphosphate into the DNA strand, if it is complementary to the base in the template strand. Each incorporation event is accompanied by release of pyrophosphate (PPi) in a quantity equimolar to the amount of incorporated nucleotide. ATP sulfurylase quantitatively converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a Pyrogram™. Each light signal is proportional to the number of nucleotides incorporated. Apyrase, a nucleotide degrading enzyme, continuously degrades unincorporated dNTPs and excess ATP. When degradation is complete, another dNTP is added.

Another similar method for characterizing SNVs does not require use of a complete PCR, but typically uses only the extension of a primer by a single, fluorescence-labeled dideoxyribonucleic acid molecule (ddNTP) that is complementary to the nucleotide to be investigated. The nucleotide at the polymorphic site can be identified via detection of a primer that has been extended by one base and is fluorescently labeled (e.g., Kobayashi et al, Mol. Cell. Probes, 9:175-182, 1995).

Additionally, SNVs can be determined from analyses (e.g., computational analyses) of data obtained from next generation sequencing (NGS) experiments Buermans and Dunnen. Biochimica et Biophysica Acta. 1842:1932-1941, 2014). Sequencing can be performed by various systems currently available, such as, without limitation, a sequencing system by ILLUMINA®, Pacific Biosciences (PACBIO®), Oxford NANOPORE®, or Life Technologies (ION TORRENT®). Methods, reagents, and equipment for performing these different sequencing systems can be obtained from their respective manufacturers. Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads. A read may include a string of nucleic acid bases corresponding to a sequence of a nucleic acid molecule that has been sequenced.

SNVs can be identified from data generated by NGS experiments by comparing the occurrence of different nucleic acid base pairs at the same locus across multiple samples. Due to errors that occur in NGS sequencing, probabilistic models (e.g. Bayesian models) are often used to distinguish and correct read errors from true SNVs. A wide variety of methods and algorithms have been developed to detect SNVs from NGS data (see, e.g., Nielsen et al. Nat. Rev. Genet. 12 (6): 443-451, 2011; Bansal, Bioinformatics. 26 (12): i318-i324, 2010; Roth et al. Bioinformatics. 28 (7): 907-913, 2012; You et al. Bioinformatics. 28 (5): 643-650, 2012; Li et al., Genome Res. 19 (6): 1124-1132, 2009; Abecasis et al. Nature. 467 (7319): 1061-1073, 2010; Larson et al. Bioinformatics. 28 (3): 311-317, 2012). Resources for identifying SNVs found in the human genome include databases of sequenced genomes (e.g., gnomAD, Bravo, ClinVar, 1000 Genome Project, and TopMed) and databases of identified SNVs (e.g., dbSNP, HapMap, Biomart, SPSmart, and Genome Variation Server (GVS)).

### ATR Inhibitors

ATR inhibitors a compound that upon contacting the enzyme ATR kinase, whether in vitro, in cell culture, or in an animal, reduces the activity of ATR kinase, such that the measured ATR kinase IC₅₀ is 10 μM or less (e.g., 5 μM or less or 1 μM or less). For certain ATR inhibitors, the ATR kinase IC₅₀ may be 100 nM or less (e.g., 10 nM or less, or 1 nM or less) and could be as low as 100 μM or 10 μM. Preferably, the ATR kinase IC₅₀ is 0.1 nM to 1 μM (e.g., 0.1 nM to 750 nM, 0.1 nM to 500 nM, or 0.1 nM to 250 nM).

Examples of ATR inhibitors are:

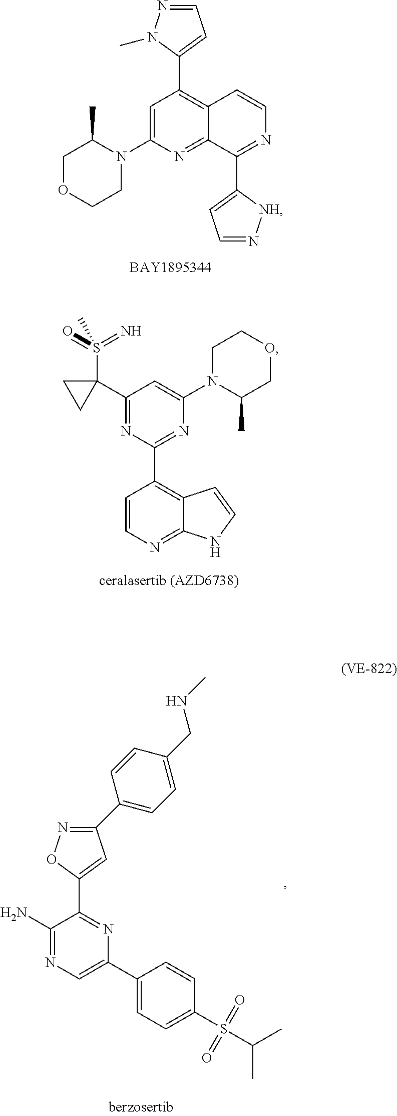 [SMILES: C[C@@H]1COCCN1c1cc(-c2ccnn2C)c2ccnc(-c3ccn[nH]3)c2n1 ; C[C@@H]1COCCN1c1cc(C2([S@](C)(=N)=O)CC2)nc(-c2ccnc3[nH]ccc23)n1 ; CNCc1ccc(-c2cc(-c3nc(-c4ccc(S(=O)(=O)C(C)C)cc4)cnc3N)on2)cc1]

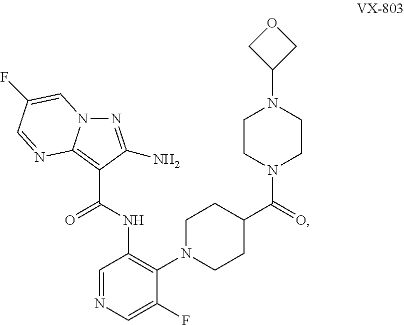 [SMILES: Nc1nn2cc(F)cnc2c1C(=O)Nc1cncc(F)c1N1CCC(C(=O)N2CCN(C3COC3)CC2)CC1]

and pharmaceutically acceptable salts thereof.

Additional examples of ATR inhibitors are those described in International Application Nos. PCT/US2019/051539 and PCT/US2018/034729; U.S. Pat. Nos. 9,663,535, 9,549,932, 8,552,004, and 8,841,308; and U.S. Patent Application Publication No. 2019/0055240, each of which is incorporated by reference herein.

In one embodiment. an ATR inhibitor is a compound of formula (I):

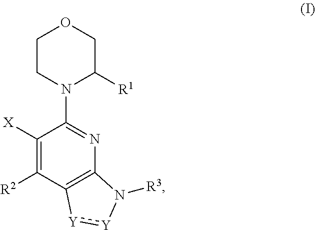

or a pharmaceutically acceptable salt thereof,
where
   [symbol] is a double bond, and each Y is independently N or CR⁴; or [symbol] is a single bond, and each Y is independently NR^{Y}, carbonyl, or C(R^{Y})₂; where each R^{Y} is independently H or optionally substituted C₁₋₆ alkyl;
   R¹ is optionally substituted C₁₋₆ alkyl or H;
   R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
   R³ is optionally substituted C₁₋₉ heteroaryl or optionally substituted C₁₋₉ heteroaryl C₁₋₆ alkyl;
   each R⁴ is independently hydrogen, halogen, optionally substituted C₁₋₆ alkyl, optionally substituted C₂₋₆ alkenyl, or optionally substituted C₂₋₆ alkynyl;
   each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
   each R^{5A} is independently optionally substituted C₁₋₆ alkyl, optionally substituted C₃₋₈ cycloalkyl, or optionally substituted C₆₋₁₀ aryl;
   R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
   each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
   Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
   X is hydrogen or halogen.

The ATR inhibitor may be, e.g., a compound of formula (II):

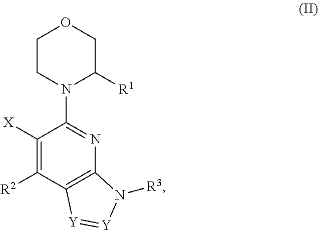

or a pharmaceutically acceptable salt thereof,
where
   each Y is independently N or CR⁴;
   R¹ is optionally substituted C₁₋₆ alkyl or H;
   R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
   R³ is optionally substituted C₁₋₉ heteroaryl or optionally substituted C₁₋₉ heteroaryl C₁₋₆ alkyl;
   each R⁴ is independently hydrogen, halogen, optionally substituted C₁₋₆ alkyl, optionally substituted C₂₋₆ alkenyl, or optionally substituted C₂₋₆ alkynyl;
   each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
   each R^{5A} is independently optionally substituted C₁₋₆ alkyl, optionally substituted C₃₋₈ cycloalkyl, or optionally substituted C₆₋₁₀ aryl;
   R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
   each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
   Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
   X is hydrogen or halogen.

In some embodiments, in the compound of formula (II), (I), or (I-b):
each Y is independently N or CR⁴;
R¹ is H or optionally substituted C₁₋₆ alkyl;
R² is optionally substituted C₁₋₆ alkyl, optionally substituted C₃₋₈ cycloalkyl, optionally substituted C₂₋₉ heterocyclyl, optionally substituted C₆₋₁₀ aryl, optionally substituted C₁₋₉ heteroaryl, optionally substituted C₁₋₉ heteroaryl C₁₋₆ alkyl, —N(R⁵)₂, —CON(R⁶)₂, —SO₂N(R⁶)₂, or —SO₂R^{5A};
R³ is optionally substituted C₁₋₉ heteroaryl;
each R⁴ is independently H or optionally substituted C₁₋₆ alkyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}, where each R^{5A} is independently optionally substituted C₁₋₆ alkyl or optionally substituted C₃₋₈ cycloalkyl; or both R⁵, together with the atom to which they are attached, combine to form an optionally substituted C₂₋₉ heterocyclyl;
each R^{5A} is independently optionally substituted C₁₋₆ alkyl or optionally substituted C₃₋₈ cycloalkyl; and
each R⁶ is independently hydrogen, optionally substituted C₁₋₆ alkyl, optionally substituted C₆₋₁₀ aryl C₁₋₆ alkyl, optionally substituted C₆₋₁₀ aryl, or optionally substituted C₁₋₉ heteroaryl; or both R⁶, together with the atom to which they are attached, combine to form an optionally substituted C₂₋₉ heterocyclyl.

Methods of making compounds of formula (I) are described, e.g., in International Application No. PCT/US2019/051539, hereby incorporated by reference.

The ATR inhibitor may be, e.g., a compound of formula (I-a):

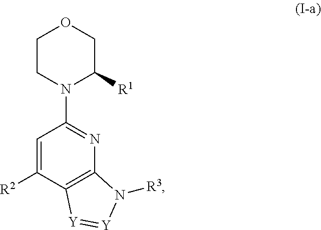

or a pharmaceutically acceptable salt thereof, where Y, R¹, R², R³, and R⁴ are as described for formula (I).

The ATR inhibitor may be, e.g., a compound of formula (I-b):

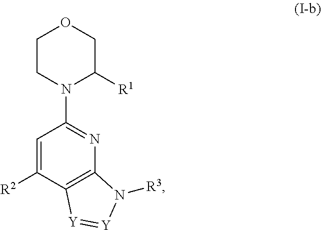

or a pharmaceutically acceptable salt thereof, where Y, R¹, R², R³, and R⁴ are as described for formula (I).

The ATR inhibitor may be, e.g., a compound of formula (IA):

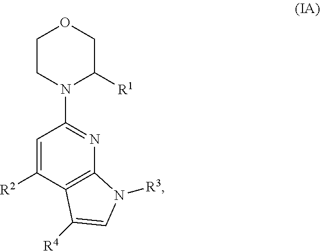

or a pharmaceutically acceptable salt thereof, where R¹, R², R³, and R⁴ are as described for formula (I).

The ATR inhibitor may be, e.g., a compound of formula (IA-a):

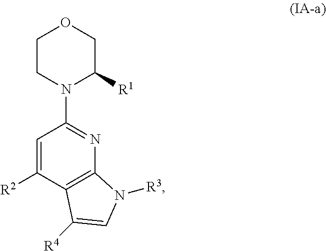

or a pharmaceutically acceptable salt thereof, where R¹, R², R³, and R⁴ are as described for formula (I).

The ATR inhibitor may be, e.g., a compound of Formula (IB):

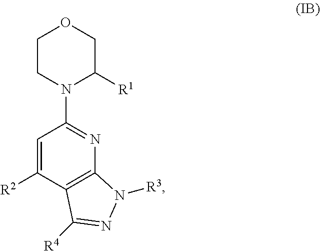

or a pharmaceutically acceptable salt thereof, where R¹, R², R³, and R⁴ are as described for formula (I).

The ATR inhibitor may be, e.g., a compound of formula (IB-a):

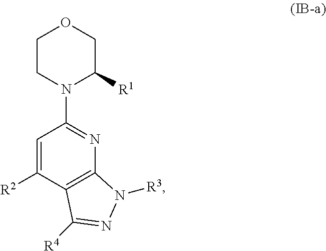

or a pharmaceutically acceptable salt thereof, where R¹, R², R³, and R⁴ are as described for formula (I).

The ATR inhibitor may be, e.g., a compound of Formula (IC):

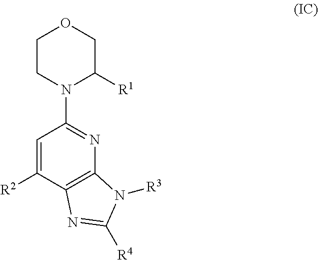

or a pharmaceutically acceptable salt thereof, where R¹, R², R³, and R⁴ are as described for formula (I).

The ATR inhibitor may be, e.g., a compound of formula (IC-a):

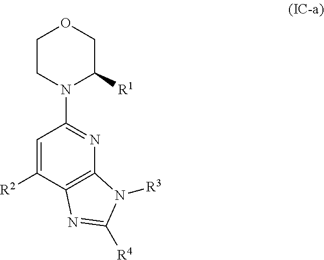

or a pharmaceutically acceptable salt thereof, where R¹, R², R³, and R⁴ are as described for formula (I).

The ATR inhibitor may be, e.g., a compound of formula (ID):

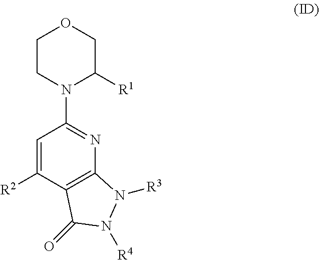

or a pharmaceutically acceptable salt thereof, where R¹, R², R³, and R⁴ are as described for formula (I).

The ATR inhibitor may be, e.g., a compound of formula (ID-a):

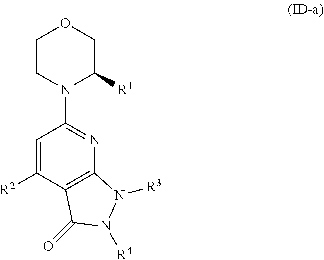

or a pharmaceutically acceptable salt thereof, where R¹, R², R³, and R⁴ are as described for formula (I).

Preferably, R¹ is methyl.

In some embodiments, R² may be, e.g., optionally substituted C₃₋₈ cycloalkyl. For example, R² may be a group of formula (A):

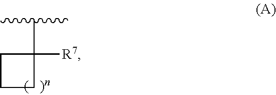

where
   n is 0, 1, 2, or 3; and
   R⁷ is hydrogen, alkylsulfonyl, cyano, —CON(R^{A})₂, —SON(R^{A})₂, optionally substituted C₁₋₉ heteroaryl, hydroxy, or alkoxy, where each R^{A} is independently H or alkyl; or both R^{A}, together with the atom to which they are attached, combine to form C₂₋₉ heterocyclyl.

In some embodiments, R² may be, e.g., optionally substituted C₁₋₆ alkyl (e.g., optionally substituted tertiary C₃₋₆ alkyl. For example, R² may be a group of formula (B):

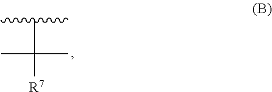

where R⁷ is hydrogen, alkylsulfonyl, cyano, —CON(R^{A})₂, —SON(R^{A})₂, optionally substituted C₁₋₉ heteroaryl, hydroxy, or alkoxy, where each R^{A} is independently H or alkyl; or both R^{A}, together with the atom to which they are attached, combine to form C₂₋₉ heterocyclyl.

In some embodiments, R² may be, e.g., optionally substituted non-aromatic C₂₋₉ heterocyclyl.

In some embodiments, R² may be, e.g.:

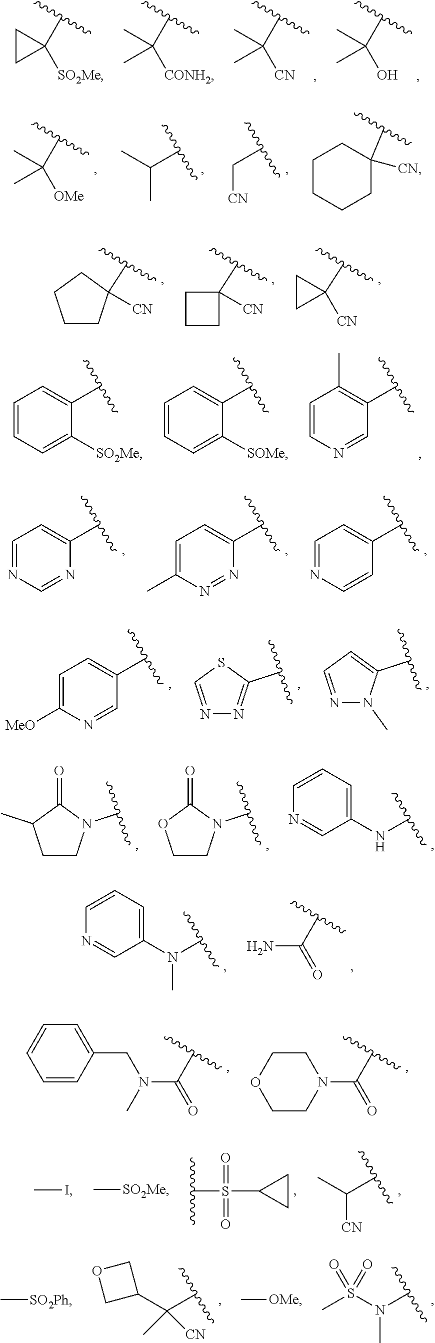

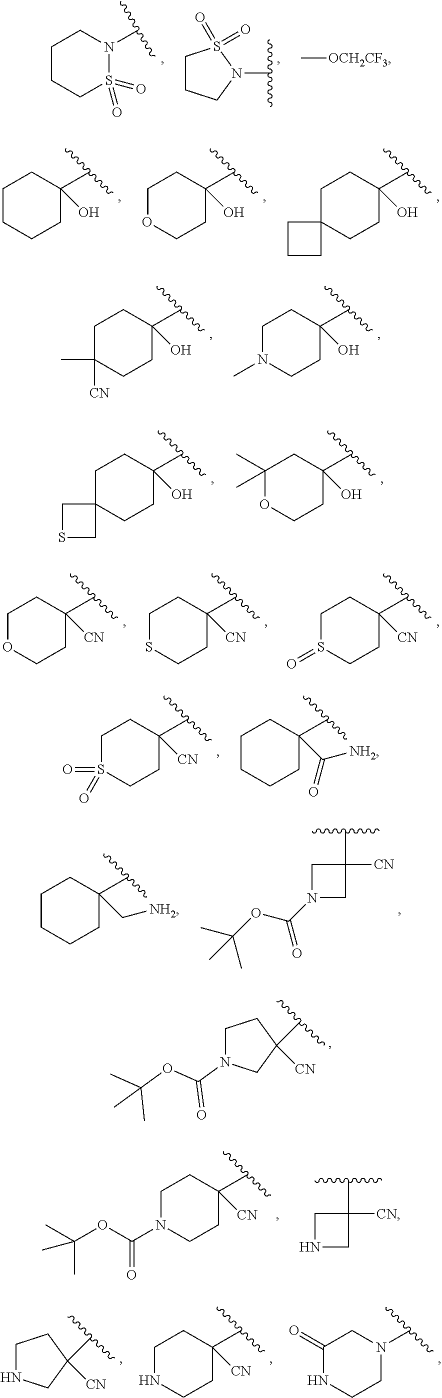

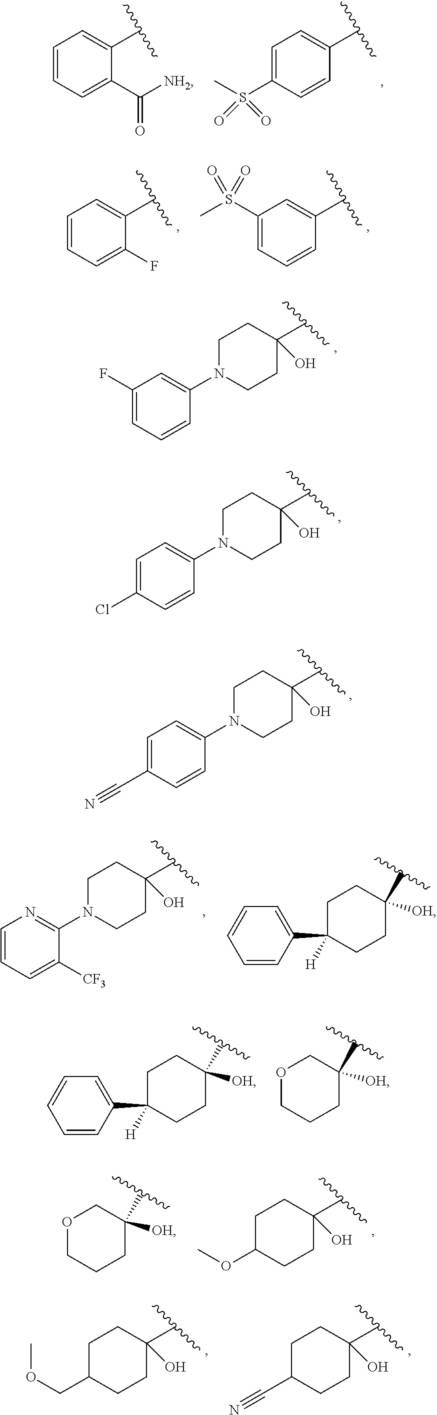

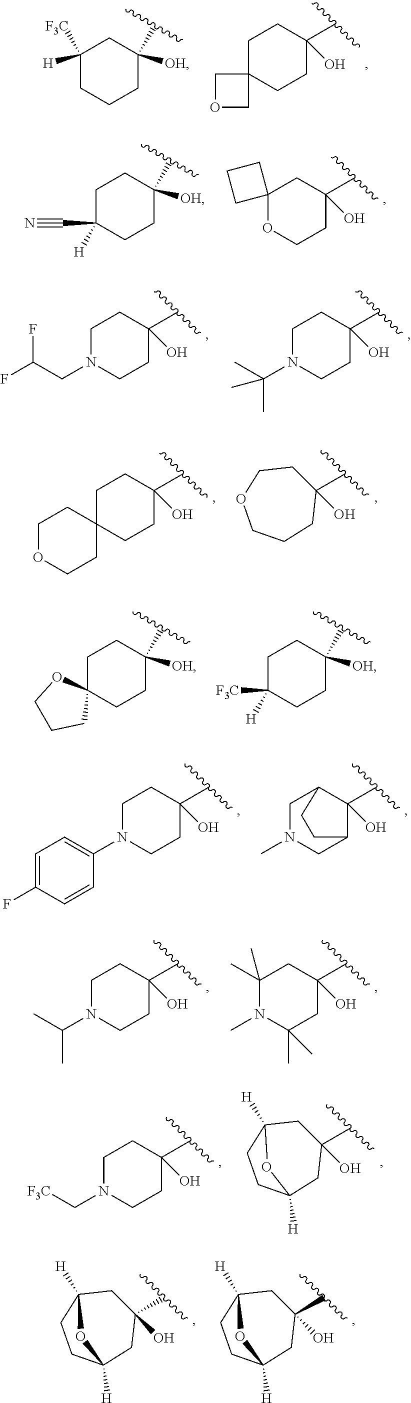

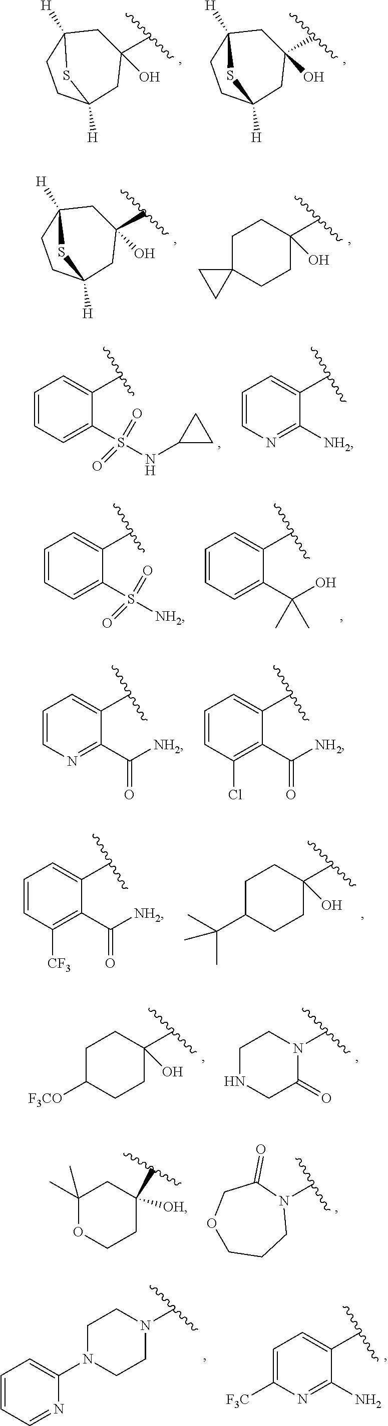

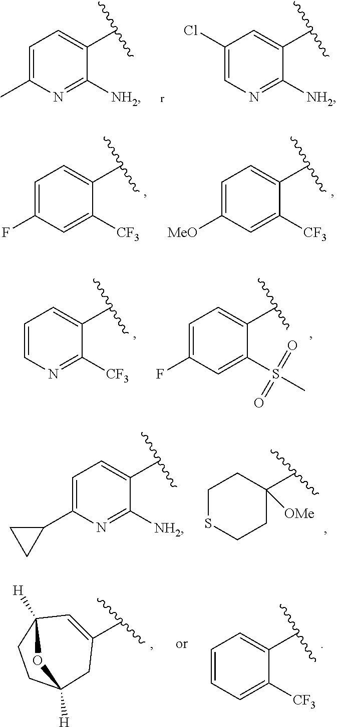

In some embodiments, R³ may be, e.g., optionally substituted, monocyclic C₁₋₉ heteroaryl including at least one nitrogen atom (e.g., two nitrogen atoms). For example, R³ may be a group of formula (C):

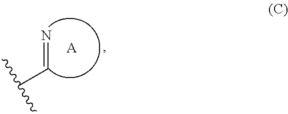

where A is optionally substituted, monocyclic C₁₋₉ heteroaryl ring.

In some embodiments, A may be, e.g., a group of formula (C1):

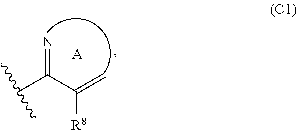

where R₈ is hydrogen, halogen, or optionally substituted Cie alkyl.

In some embodiments, R³ may be, e.g.:

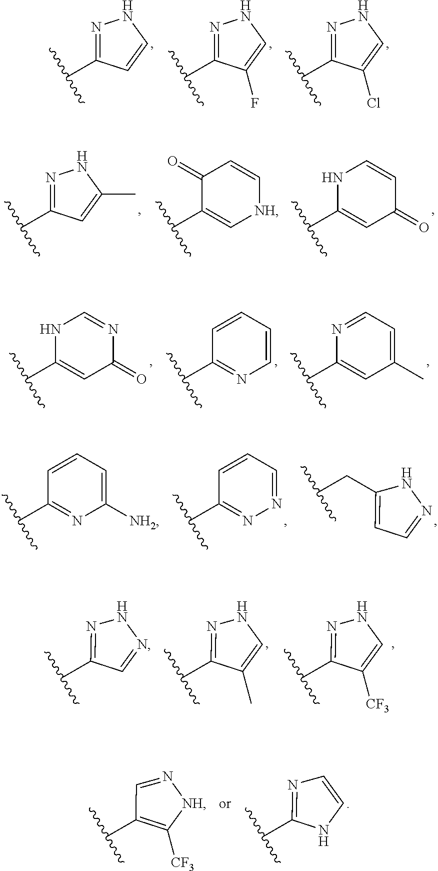

In some embodiments, R³ may be, e.g.:

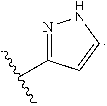 [SMILES: *c1cc[nH]n1]

In some embodiments, R⁴ may be, e.g., hydrogen.

The ATR inhibitor may be, e.g., a compound listed in Table 1 or a pharmaceutically acceptable salt thereof.

TABLE 1

| | |
|---|---|
| 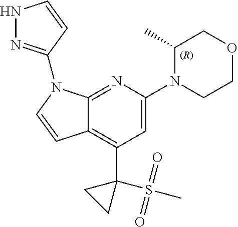 | 1 |
| 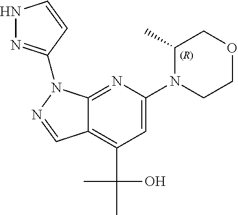 | 2 |
| 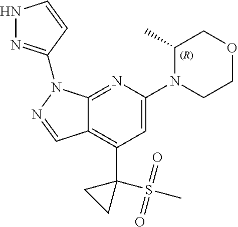 | 3 |
| 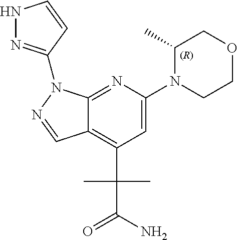 | 4 |
| 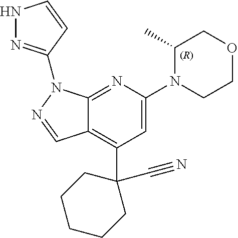 | 5 |
| 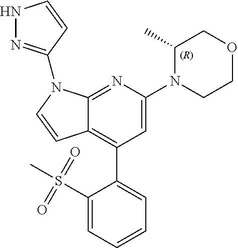 | 6 |
| 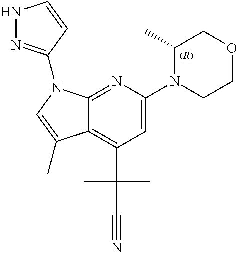 | 7 |
| 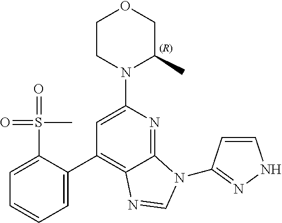 | 8 |
| 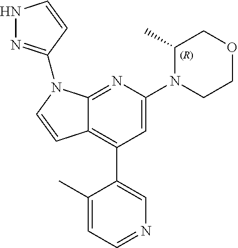 | 9 |
| 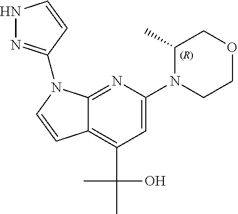 | 10 |
| 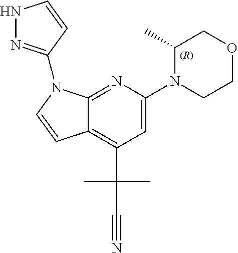 | 11 |
| 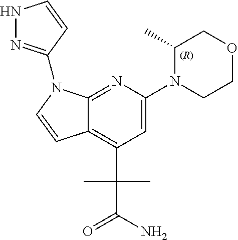 | 12 |
| 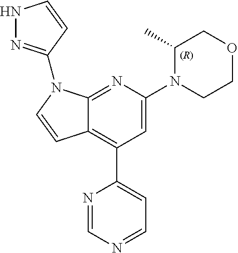 | 13 |
| 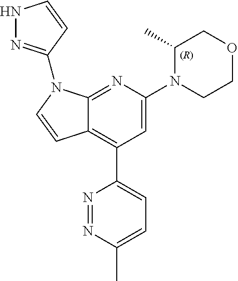 | 14 |
| 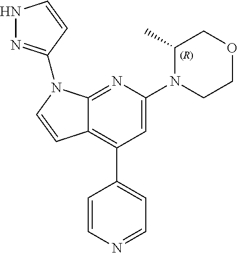 | 15 |
| 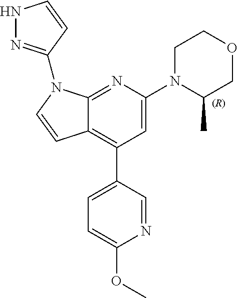 | 16 |
| 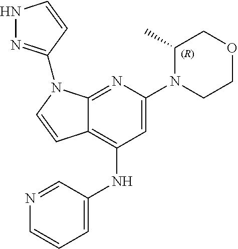 | 17 |
| 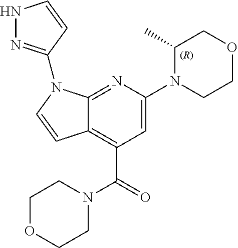 | 18 |
| 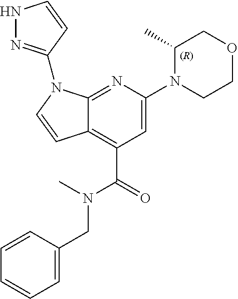 | 19 |
| 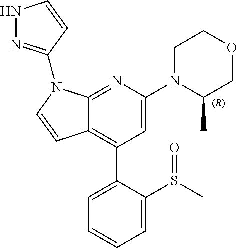 | 20 |
| 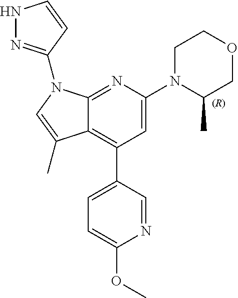 | 21 |
| 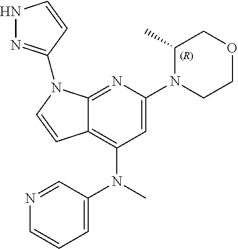 | 22 |
| 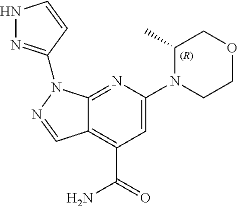 | 23 |
| 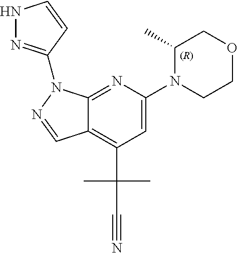 | 24 |
| 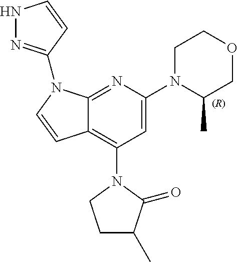 | 25 |
| 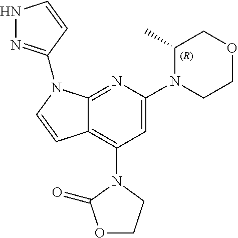 | 26 |
| 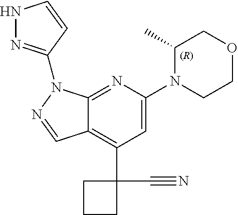 | 27 |
| 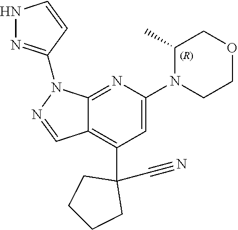 | 28 |
| 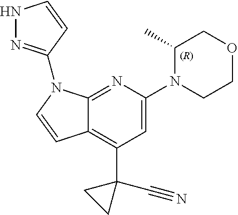 | 29 |
| 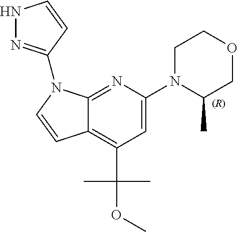 | 30 |
| 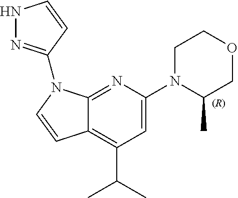 | 31 |
| 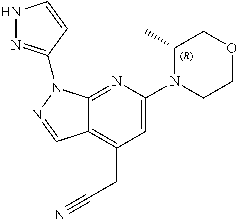 | 32 |
| 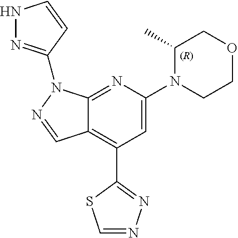 | 33 |
| 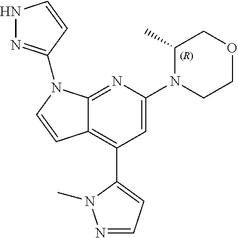 | 34 |
| 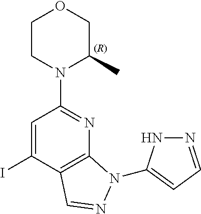 | 42 |
| 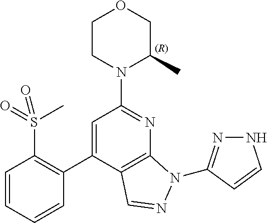 | 43 |
| 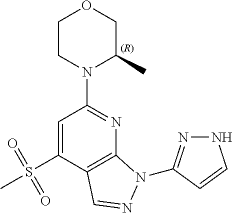 | 44 |
| 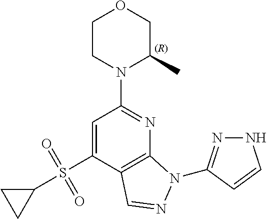 | 45 |
| 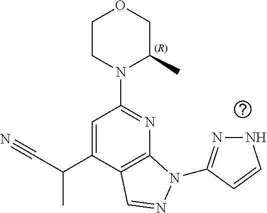 | 47 |
| 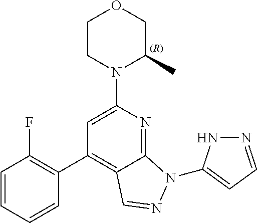 | 48 |
| 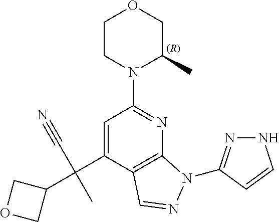 | 49 |
| 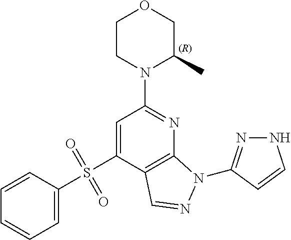 | 50 |
| 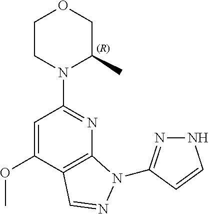 | 51 |
| 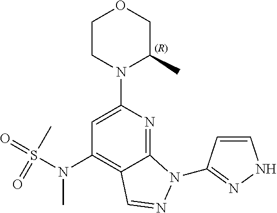 | 52 |
| 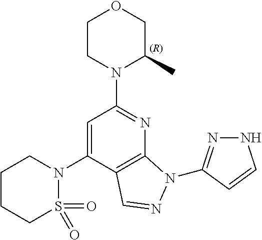 | 53 |
| 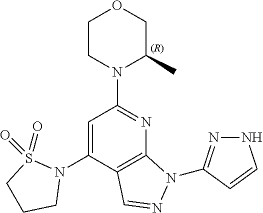 | 54 |
| 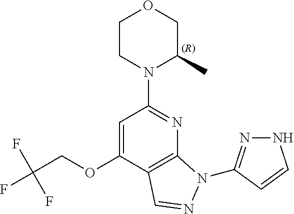 | 55 |
| 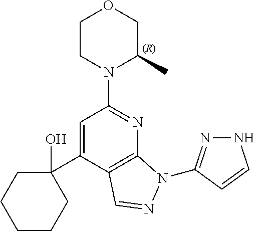 | 56 |
| 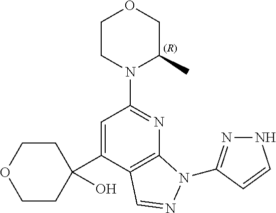 | 57 |
| 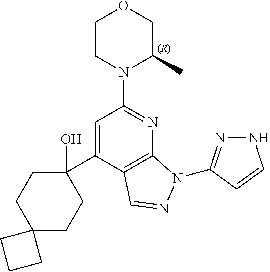 | 58 |
| 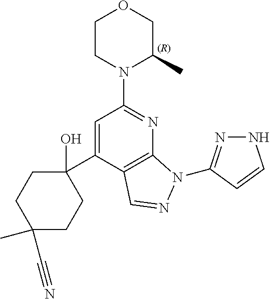 | 59 |
| 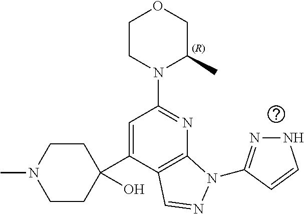 | 60 |
| 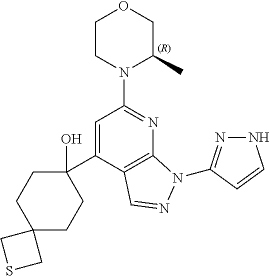 | 61 |
| 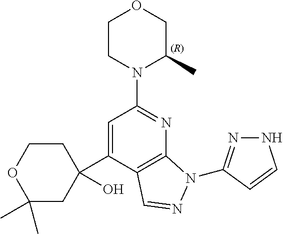 | 62 |
| 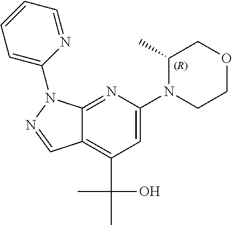 | 63 |
| 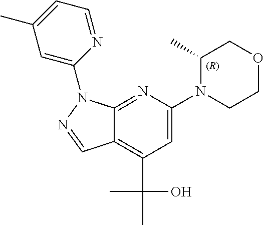 | 64 |
| 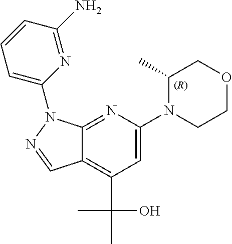 | 65 |
| 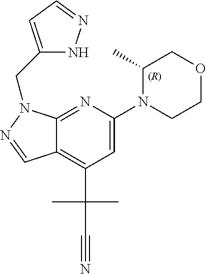 | 66 |
| 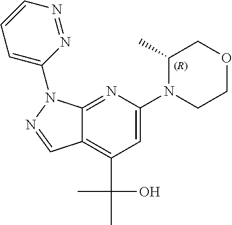 | 67 |
| 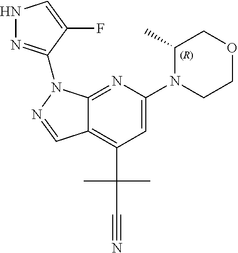 | 68 |
| 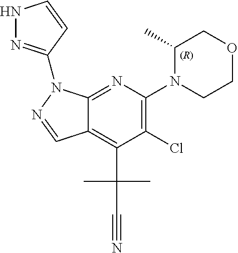 | 69 |
| 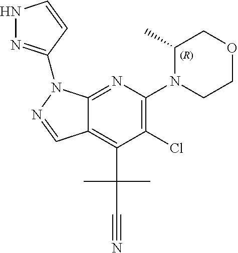 | 70 |
| 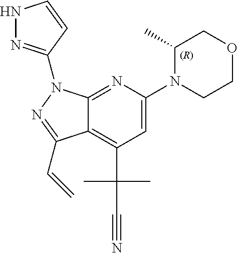 | 71 |
| 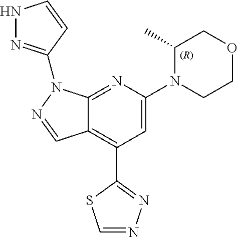 | 72 |
| 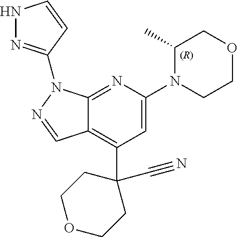 | 73 |
| 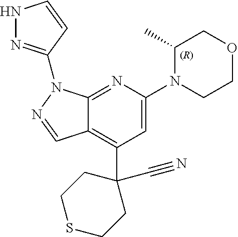 | 74 |
| 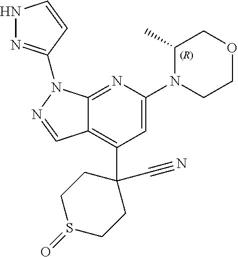 | 75 |
| 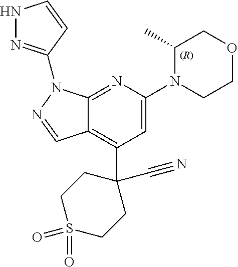 | 76 |
| 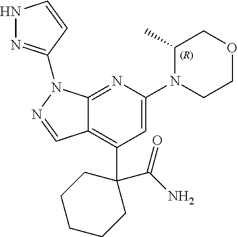 | 77 |
| 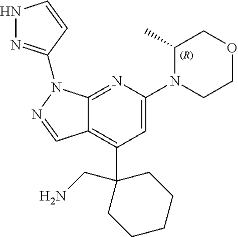 | 78 |
| 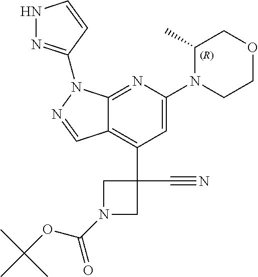 | 79 |
| 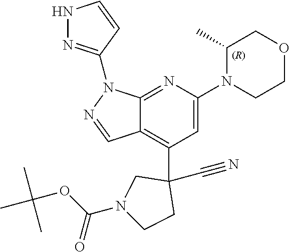 | 80 |
| 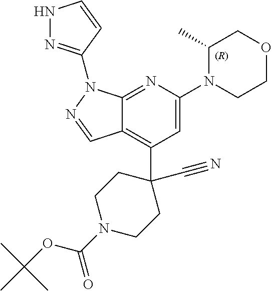 | 81 |
| 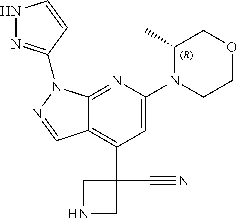 | 82 |
| 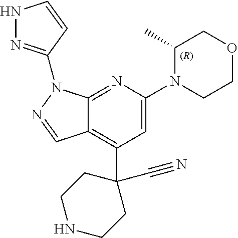 | 83 |
| 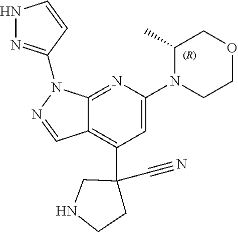 | 84 |
| 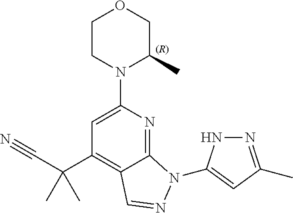 | 86 |
| 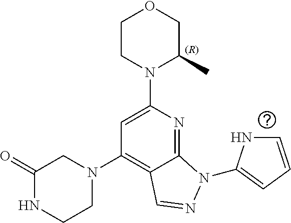 | 87 |
| 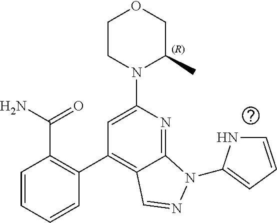 | 88 |
| 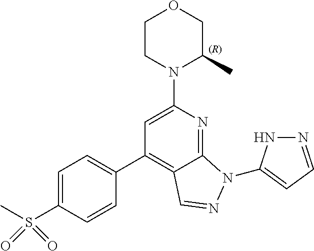 | 89 |
| 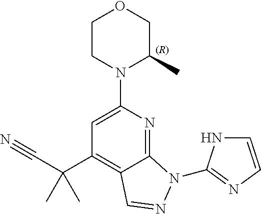 | 90 |
| 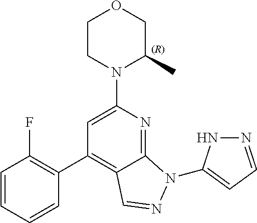 | 91 |
| 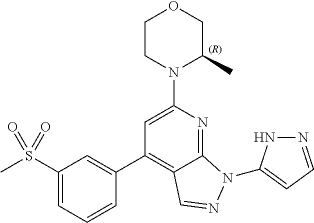 | 92 |
| 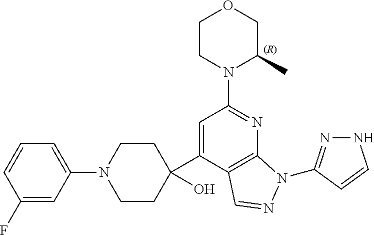 | 93 |
| 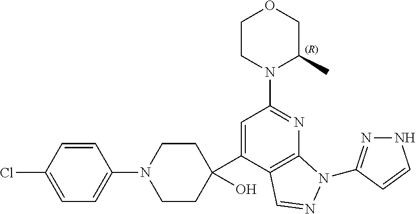 | 94 |
| 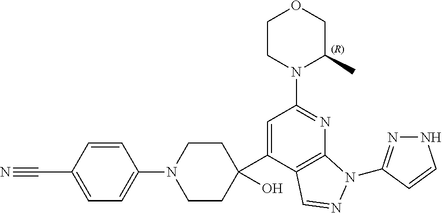 | 95 |
| 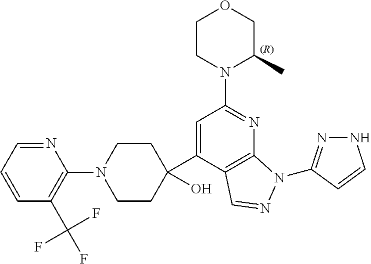 | 96 |
| 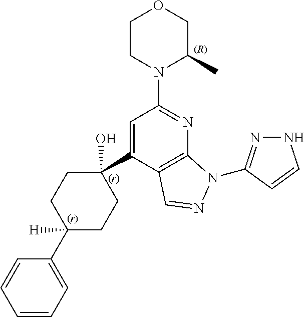 | 97 |
| 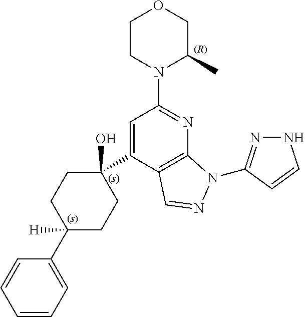 | 98 |
| 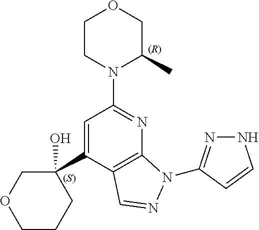 | 99 |
| 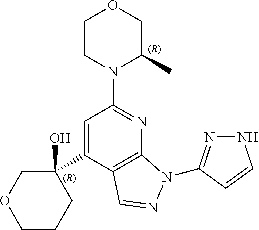 | 100 |
| 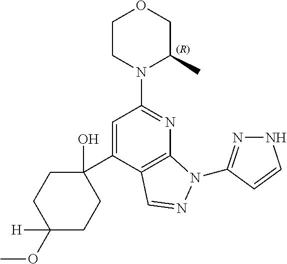 | 101 |
| 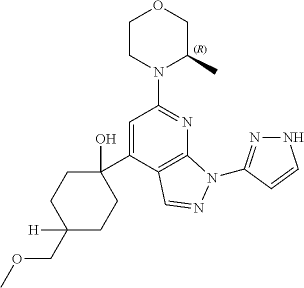 | 102 |
| 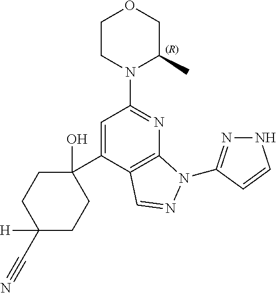 | 103 |
| 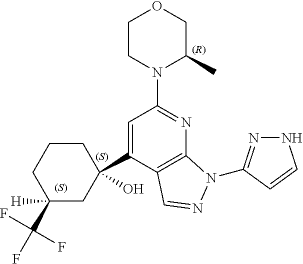 | 104 |
| 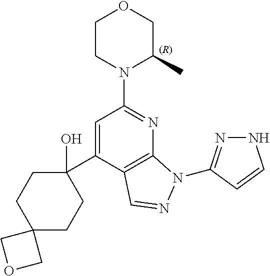 | 105 |
| 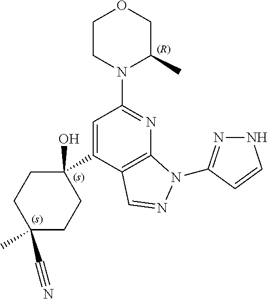 | 106 |
| 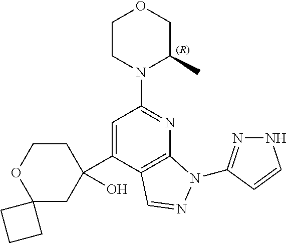 | 107 |
| 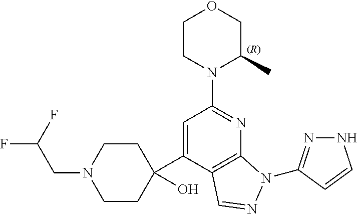 | 108 |
| 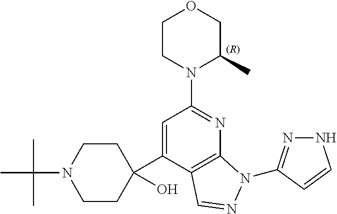 | 109 |
| 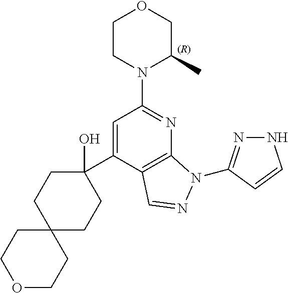 | 110 |
| 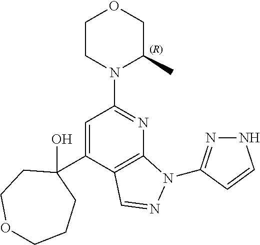 | 111 |
| 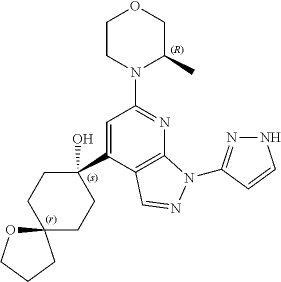 | 112 |
| 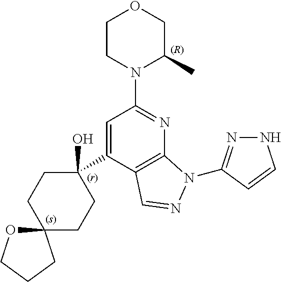 | 113 |
| 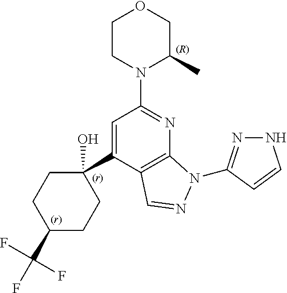 | 114 |
| 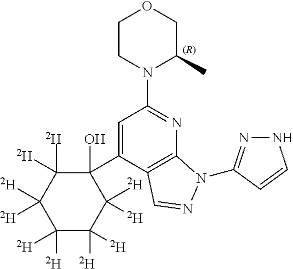 | 115 |
| 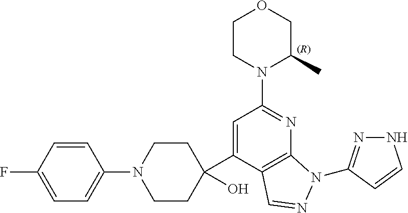 | 116 |
| 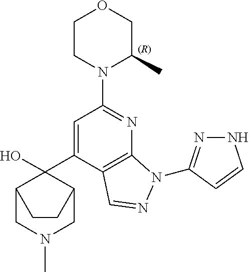 | 117 |
| 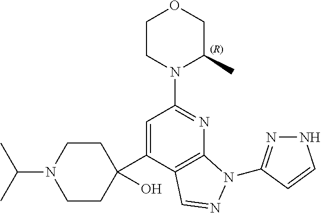 | 118 |
| 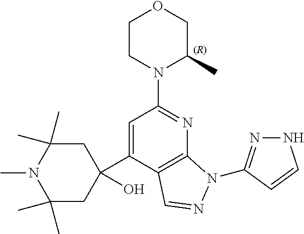 | 119 |
| 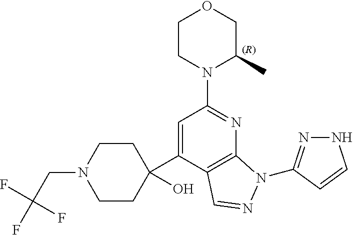 | 120 |
| 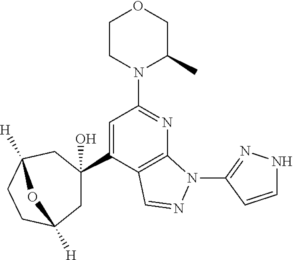 | 121 |
| 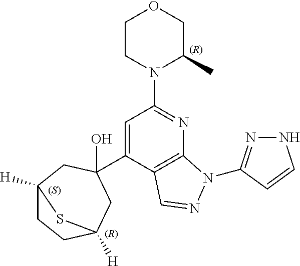 | 122 |
| 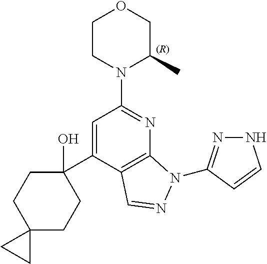 | 123 |
| 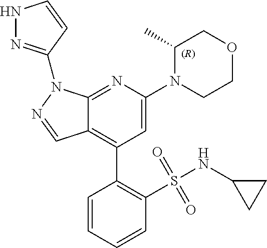 | 124 |
| 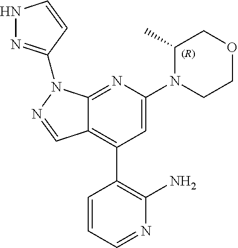 | 125 |
| 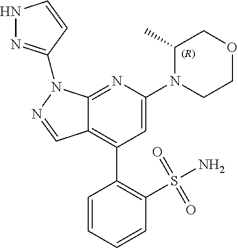 | 126 |
| 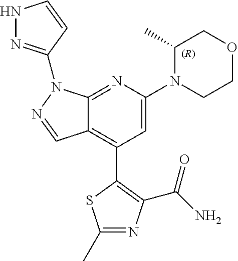 | 127 |
| 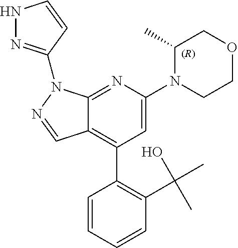 | 128 |
| 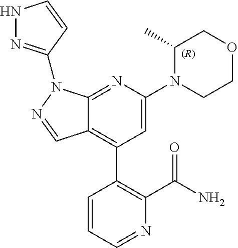 | 129 |
| 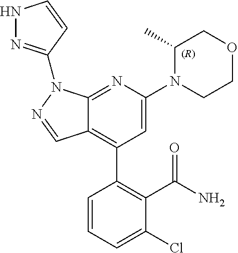 | 130 |
| 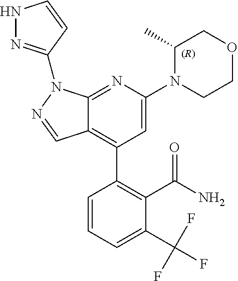 | 131 |
| 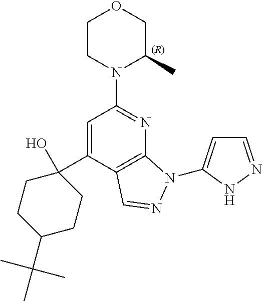 | 132 |
| 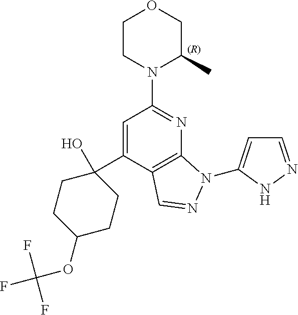 | 133 |
| 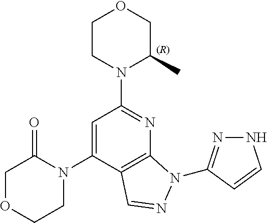 | 134 |
| 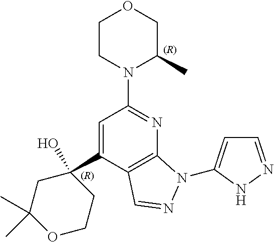 | 135 |
| 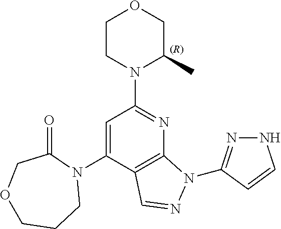 | 136 |
| 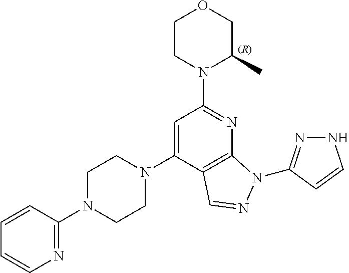 | 137 |
| 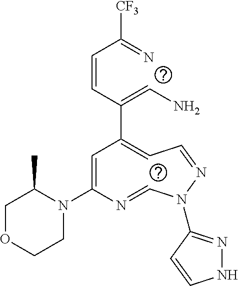 [SMILES: C[C@@H]1COCCN1c1cc(C(/C=C\C(=N)C(F)(F)F)=C\N)ccnn(-c2cc[nH]n2)cn1] | 138 |
| 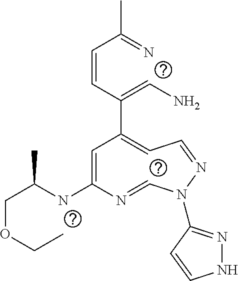 [SMILES: CCOC[C@@H](C)Nc1cc(C(/C=C\C(C)=N)=C\N)ccnn(-c2cc[nH]n2)cn1] | 139 |
| 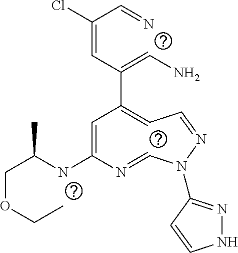 [SMILES: CCOC[C@@H](C)Nc1cc(C(=C\N)/C=C(/Cl)C=N)ccnn(-c2cc[nH]n2)cn1] | 140 |
| 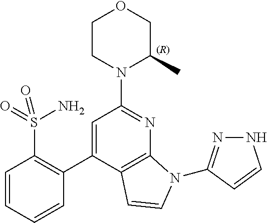 | 141 |
| 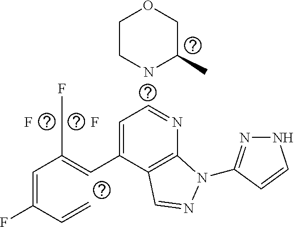 | 142 |
| 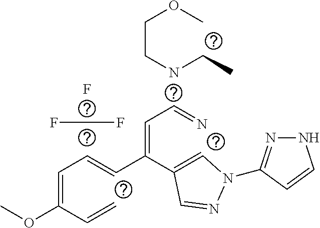 | 143 |
| 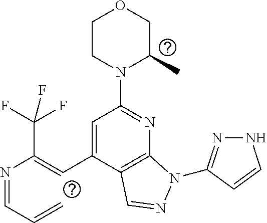 [SMILES: C=C/C=N\C(=C\c1cc(N2CCOC[C@H]2C)nc2c1cnn2-c1cc[nH]n1)C(F)(F)F] | 144 |
| 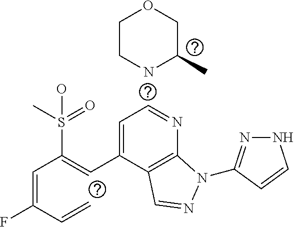 | 145 |
| 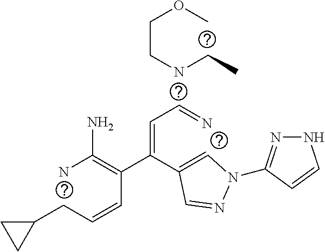 | 146 |
| 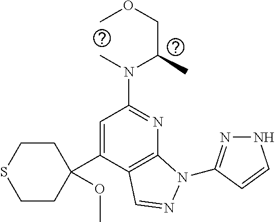 [SMILES: COC[C@@H](C)N(C)c1cc(C2(OC)CCSCC2)c2cnn(-c3cc[nH]n3)c2n1] | 147 |
| 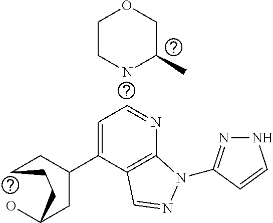 | 148 |
| 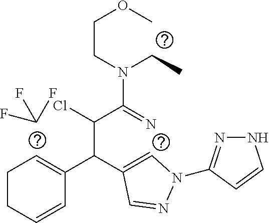 | 149 |
| 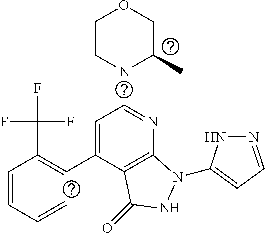 | 150 |
| 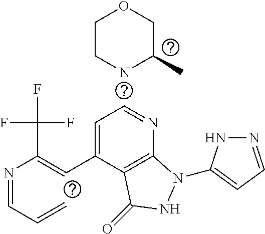 | 151 |
| 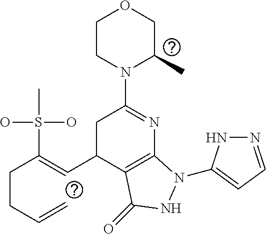 [SMILES: C=CCC/C(=C/C1CC(N2CCOC[C@H]2C)=Nc2c1c(=O)[nH]n2-c1ccn[nH]1)S(C)(O)O] | 152 |

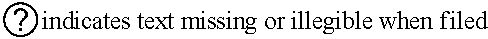

An ATR inhibitor may be isotopically enriched (e.g., enriched for deuterium).

Methods disclosed herein utilizing an ATR inhibitor may further include the use of a PARP inhibitor (e.g., administration of the PARP inhibitor to a subject). Non-limiting examples of PARP inhibitors include AZD5305, olaparib, rucaparib, veliparib (ABT-888), niraparib (ZL-2306), iniparib (BSI-201), talazoparib (BMN 673), 2X-121, CEP-9722, KU-0059436 (AZD2281), PF-01367338, pharmaceutically acceptable salts thereof, and combinations thereof.

### Myt1 Inhibitors

Myt1 inhibitors are known in the art, e.g., in WO 2021/195781 and WO 2021/195782. The Myt1 inhibitor used in the methods of the invention may be, e.g., a compound of formula (III):

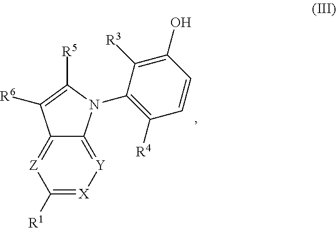

or a pharmaceutically acceptable salt thereof,
where
   each of X, Y, and Z is independently N or CR²;
   R¹ and each R² are independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, cyano, —N(R⁷)₂, —OR⁷, —C(O)N(R⁸)₂, —SO₂N(R⁸)₂, —SO₂R^{7A}, or -Q-R^{7B}; or R¹ combines with one R² that is vicinal to R¹ to form an optionally substituted C₃₋₆ alkylene;
   each of R³ and R⁴ is independently optionally substituted C₁₋₆ alkyl or halogen;
   R⁵ is H or —N(R⁷)₂;
   R⁶ is —C(O)NH(R⁸), —C(O) R^{7A}, or —SO₂R^{7A};
   each R⁷ is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or —SO₂R^{7A}; or two R⁷ groups, together with the atom to which both are attached, combine to form an optionally substituted C₂₋₉ heterocyclyl;
   each R^{7A} is independently optionally substituted C₁₋₆ alkyl, optionally substituted C₃₋₈ cycloalkyl, or optionally substituted C₆₋₁₀ aryl;
   each R^{7B} is independently hydroxyl, optionally substituted C₁₋₆ alkyl, optionally substituted C₆₋₁₀ aryl, optionally substituted C₂₋₉ heterocyclyl, optionally substituted C₁₋₉ heteroaryl, —N(R⁷)₂, —C(O)N(R⁸)₂, —SO₂N(R⁸)₂, —SO₂R^{7A}, or optionally substituted alkoxy;
   each R⁸ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or two R⁸, together with the atom to which they are attached, combine to form an optionally substituted C₂₋₉ heterocyclyl; and
   Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene optionally substituted C₆₋₁₀ arylene, optionally substituted C₂₋₉ heterocyclylene, or optionally substituted C₁₋₉ heteroarylene.

Preferably, the compound of formula (III) is enriched for the atropisomer of formula (IIIA):

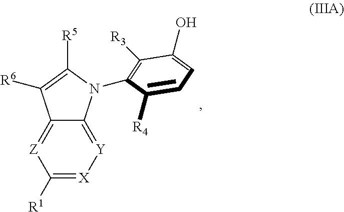

where all variables are as described herein.

The compound used in the methods of the invention may be, e.g., a compound of formula

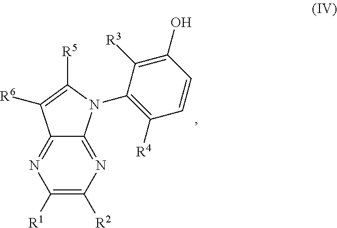

where all variables are as described herein.

Preferably, the compound of formula (IV) is enriched for the atropisomer of of formula (IVA):

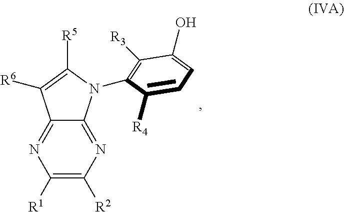

where all variables are as described herein.

The compound used in the methods of the invention may be, e.g., a compound of formula (V):

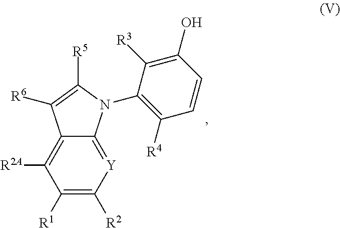

where R^{2A} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁷)₂, —OR⁷, —C(O) N(R⁸)₂, —SO₂N(R⁸)₂, —SO₂R^{7A}, or -Q-R^{7B}.

Preferably, the compound of formula (V) is enriched for the atropisomer of formula (VA):

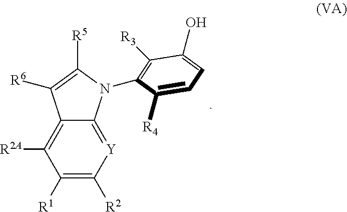

The compound used in the methods of the invention may be, e.g., a compound listed in Table 2 below or a pharmaceutically acceptable salt thereof.

TABLE 2

| | |
|---|---|
| 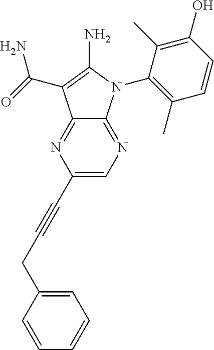 [SMILES: Cc1ccc(O)c(C)c1-n1c(N)c(C(N)=O)c2nc(C#CCc3ccccc3)cnc21] | 1 |
| 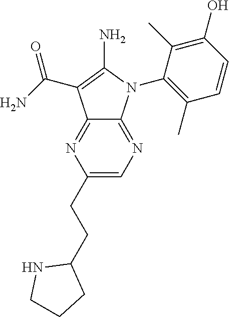 [SMILES: Cc1ccc(O)c(C)c1-n1c(N)c(C(N)=O)c2nc(CCC3CCCN3)cnc21] | 2 |
| 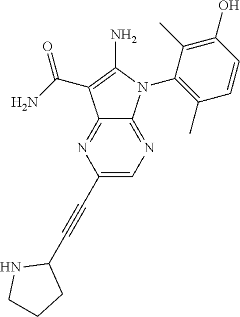 [SMILES: Cc1ccc(O)c(C)c1-n1c(N)c(C(N)=O)c2nc(C#CC3CCCN3)cnc21] | 3 |
| 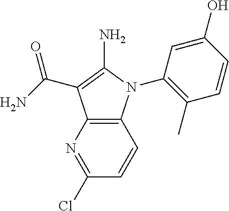 [SMILES: Cc1ccc(O)cc1-n1c(N)c(C(N)=O)c2nc(Cl)ccc21] | 4 |
| 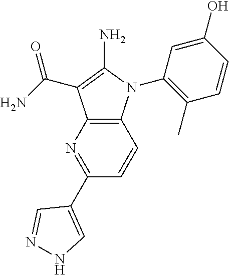 [SMILES: Cc1ccc(O)cc1-n1c(N)c(C(N)=O)c2nc(-c3cn[nH]c3)ccc21] | 5 |
| 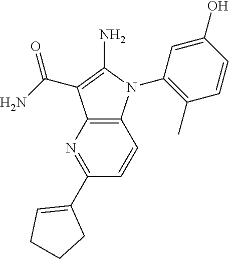 [SMILES: Cc1ccc(O)cc1-n1c(N)c(C(N)=O)c2nc(C3=CCCC3)ccc21] | 6 |
| 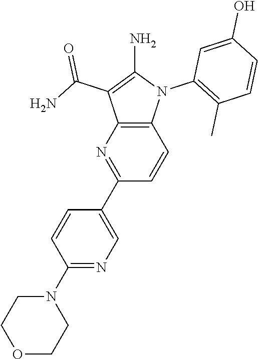 [SMILES: Cc1ccc(O)cc1-n1c(N)c(C(N)=O)c2nc(-c3ccc(N4CCOCC4)nc3)ccc21] | 7 |
| 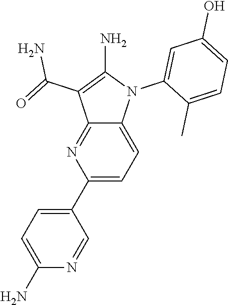 [SMILES: Cc1ccc(O)cc1-n1c(N)c(C(N)=O)c2nc(-c3ccc(N)nc3)ccc21] | 8 |
| 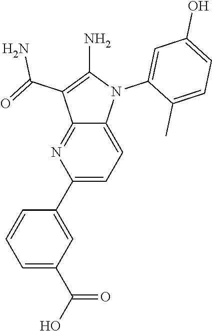 [SMILES: Cc1ccc(O)cc1-n1c(N)c(C(N)=O)c2nc(-c3cccc(C(=O)O)c3)ccc21] | 9 |
| 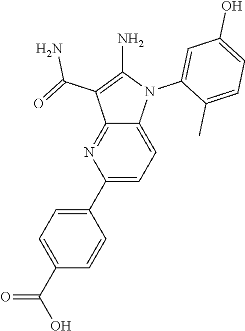 [SMILES: Cc1ccc(O)cc1-n1c(N)c(C(N)=O)c2nc(-c3ccc(C(=O)O)cc3)ccc21] | 10 |
| 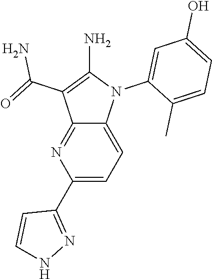 [SMILES: Cc1ccc(O)cc1-n1c(N)c(C(N)=O)c2nc(-c3cc[nH]n3)ccc21] | 11 |
| 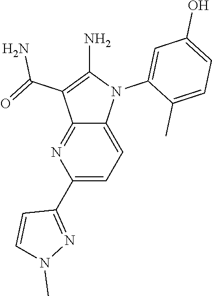 [SMILES: Cc1ccc(O)cc1-n1c(N)c(C(N)=O)c2nc(-c3ccn(C)n3)ccc21] | 12 |
| 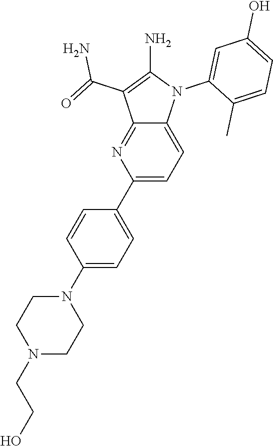 [SMILES: Cc1ccc(O)cc1-n1c(N)c(C(N)=O)c2nc(-c3ccc(N4CCN(CCO)CC4)cc3)ccc21] | 13 |
| 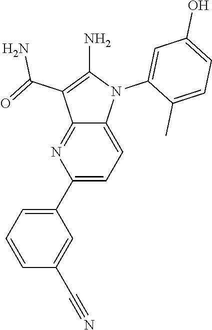 [SMILES: Cc1ccc(O)cc1-n1c(N)c(C(N)=O)c2nc(-c3cccc(C#N)c3)ccc21] | 14 |
| 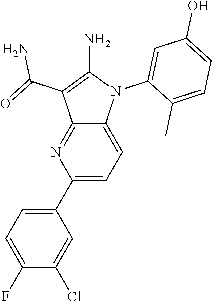 [SMILES: Cc1ccc(O)cc1-n1c(N)c(C(N)=O)c2nc(-c3ccc(F)c(Cl)c3)ccc21] | 15 |
| 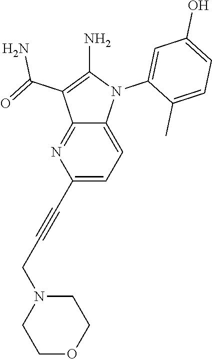 [SMILES: Cc1ccc(O)cc1-n1c(N)c(C(N)=O)c2nc(C#CCN3CCOCC3)ccc21] | 16 |
| 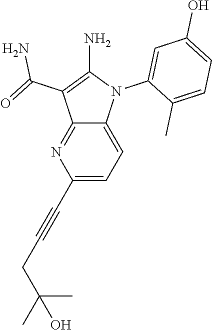 [SMILES: Cc1ccc(O)cc1-n1c(N)c(C(N)=O)c2nc(C#CCC(C)(C)O)ccc21] | 17 |
| 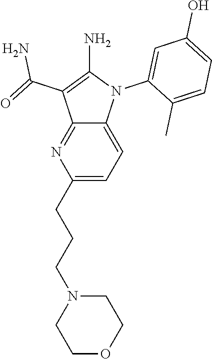 [SMILES: Cc1ccc(O)cc1-n1c(N)c(C(N)=O)c2nc(CCCN3CCOCC3)ccc21] | 18 |
| 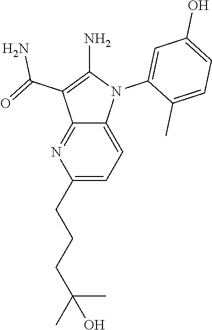 [SMILES: Cc1ccc(O)cc1-n1c(N)c(C(N)=O)c2nc(CCCC(C)(C)O)ccc21] | 19 |
| 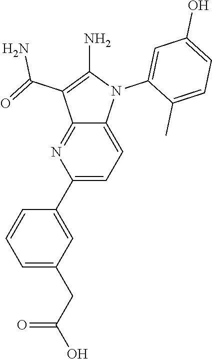 [SMILES: Cc1ccc(O)cc1-n1c(N)c(C(N)=O)c2nc(-c3cccc(CC(=O)O)c3)ccc21] | 20 |
| 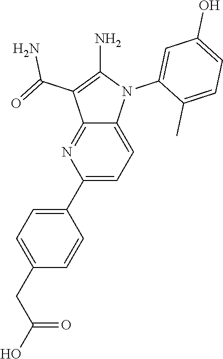 [SMILES: Cc1ccc(O)cc1-n1c(N)c(C(N)=O)c2nc(-c3ccc(CC(=O)O)cc3)ccc21] | 21 |
| 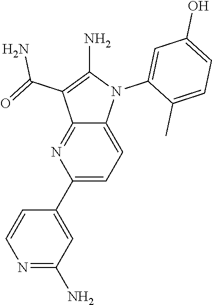 [SMILES: Cc1ccc(O)cc1-n1c(N)c(C(N)=O)c2nc(-c3ccnc(N)c3)ccc21] | 22 |
| 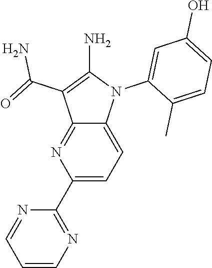 [SMILES: Cc1ccc(O)cc1-n1c(N)c(C(N)=O)c2nc(-c3ncccn3)ccc21] | 23 |
| 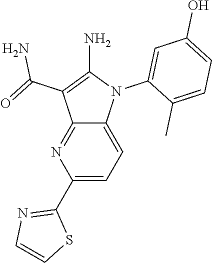 [SMILES: Cc1ccc(O)cc1-n1c(N)c(C(N)=O)c2nc(-c3nccs3)ccc21] | 24 |
| 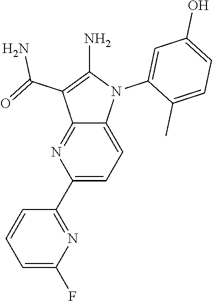 [SMILES: Cc1ccc(O)cc1-n1c(N)c(C(N)=O)c2nc(-c3cccc(F)n3)ccc21] | 25 |
| 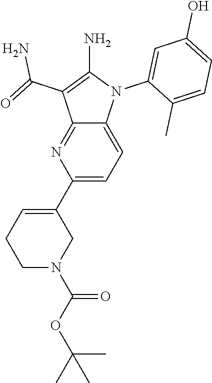 [SMILES: Cc1ccc(O)cc1-n1c(N)c(C(N)=O)c2nc(C3=CCCN(C(=O)OC(C)(C)C)C3)ccc21] | 26 |
| 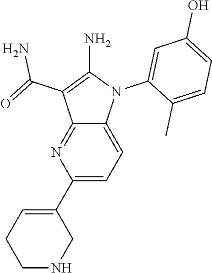 [SMILES: Cc1ccc(O)cc1-n1c(N)c(C(N)=O)c2nc(C3=CCCNC3)ccc21] | 27 |
| 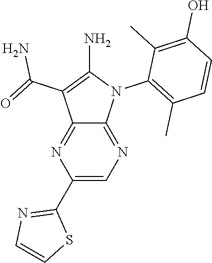 [SMILES: Cc1ccc(O)c(C)c1-n1c(N)c(C(N)=O)c2nc(-c3nccs3)cnc21] | 28 |
| 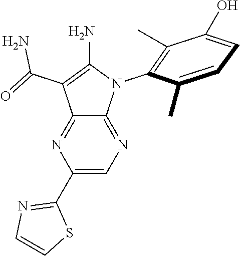 | 29 |
| 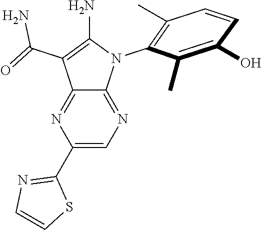 | 30 |
| 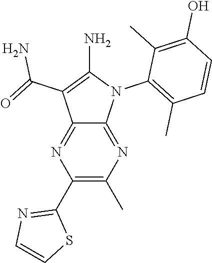 [SMILES: Cc1ccc(O)c(C)c1-n1c(N)c(C(N)=O)c2nc(-c3nccs3)c(C)nc21] | 31 |
| 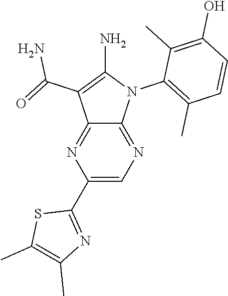 [SMILES: Cc1ccc(O)c(C)c1-n1c(N)c(C(N)=O)c2nc(-c3nc(C)c(C)s3)cnc21] | 32 |
| 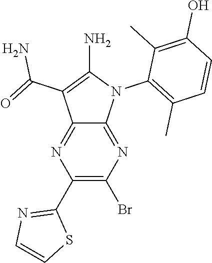 [SMILES: Cc1ccc(O)c(C)c1-n1c(N)c(C(N)=O)c2nc(-c3nccs3)c(Br)nc21] | 33 |
| 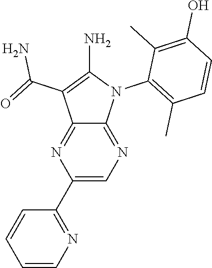 [SMILES: Cc1ccc(O)c(C)c1-n1c(N)c(C(N)=O)c2nc(-c3ccccn3)cnc21] | 34 |
| 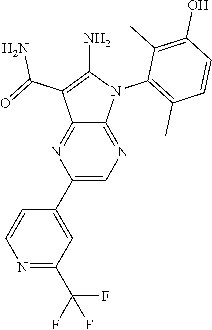 [SMILES: Cc1ccc(O)c(C)c1-n1c(N)c(C(N)=O)c2nc(-c3ccnc(C(F)(F)F)c3)cnc21] | 35 |
| 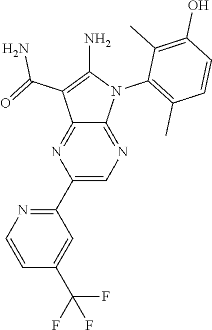 [SMILES: Cc1ccc(O)c(C)c1-n1c(N)c(C(N)=O)c2nc(-c3cc(C(F)(F)F)ccn3)cnc21] | 36 |
| 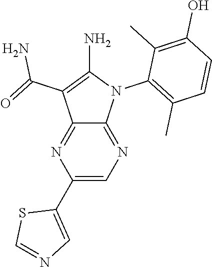 [SMILES: Cc1ccc(O)c(C)c1-n1c(N)c(C(N)=O)c2nc(-c3cncs3)cnc21] | 37 |
| 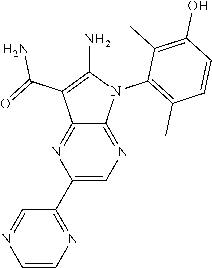 [SMILES: Cc1ccc(O)c(C)c1-n1c(N)c(C(N)=O)c2nc(-c3cnccn3)cnc21] | 38 |
| 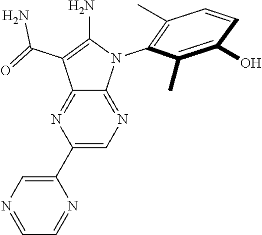 | 39 |
| 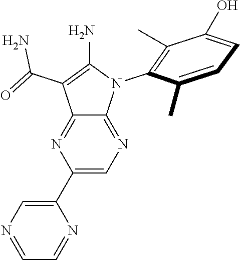 | 40 |
| 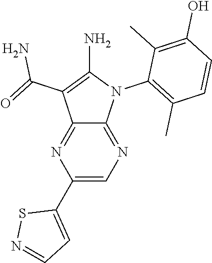 [SMILES: Cc1ccc(O)c(C)c1-n1c(N)c(C(N)=O)c2nc(-c3ccns3)cnc21] | 41 |
| 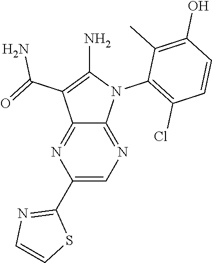 [SMILES: Cc1c(O)ccc(Cl)c1-n1c(N)c(C(N)=O)c2nc(-c3nccs3)cnc21] | 42 |
| 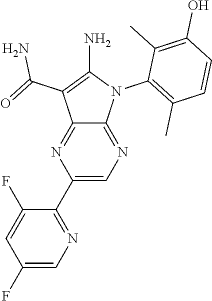 [SMILES: Cc1ccc(O)c(C)c1-n1c(N)c(C(N)=O)c2nc(-c3ncc(F)cc3F)cnc21] | 43 |
| 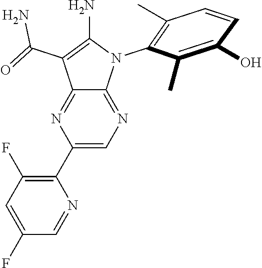 | 44 |
| 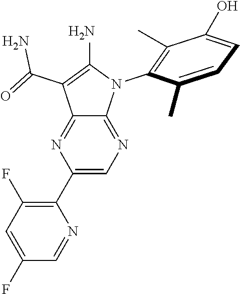 | 45 |
| 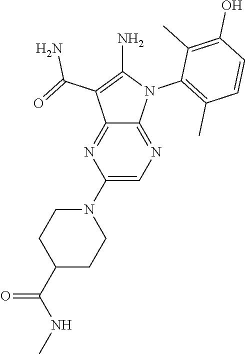 [SMILES: CNC(=O)C1CCN(c2cnc3c(n2)c(C(N)=O)c(N)n3-c2c(C)ccc(O)c2C)CC1] | 46 |
| 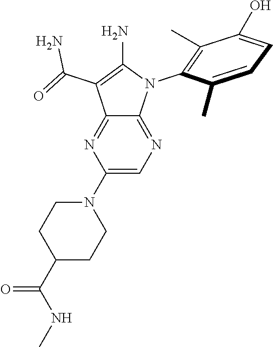 | 47 |
| 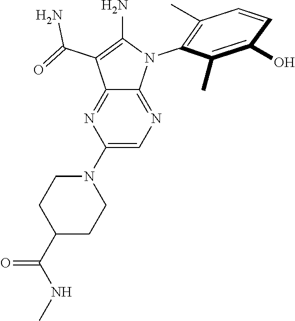 | 48 |
| 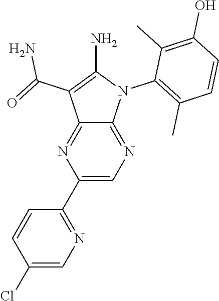 [SMILES: Cc1ccc(O)c(C)c1-n1c(N)c(C(N)=O)c2nc(-c3ccc(Cl)cn3)cnc21] | 49 |
| 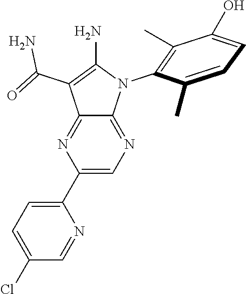 | 50 |
| 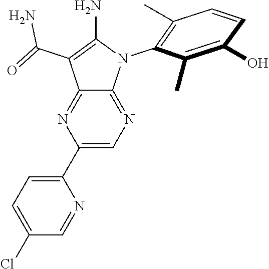 | 51 |
| 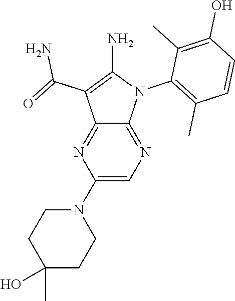 [SMILES: Cc1ccc(O)c(C)c1-n1c(N)c(C(N)=O)c2nc(N3CCC(C)(O)CC3)cnc21] | 52 |
| 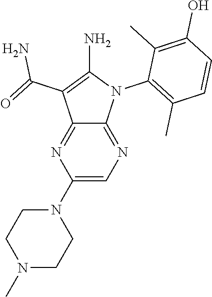 [SMILES: Cc1ccc(O)c(C)c1-n1c(N)c(C(N)=O)c2nc(N3CCN(C)CC3)cnc21] | 53 |
| 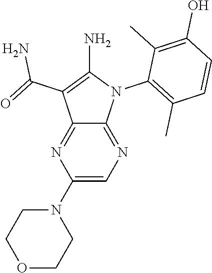 [SMILES: Cc1ccc(O)c(C)c1-n1c(N)c(C(N)=O)c2nc(N3CCOCC3)cnc21] | 54 |
| 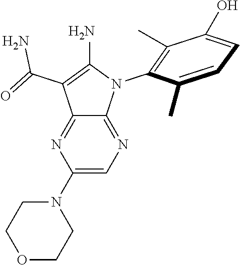 | 55 |
| 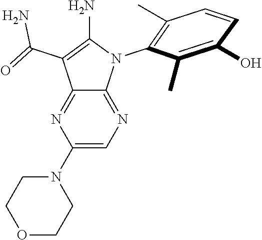 | 56 |
| 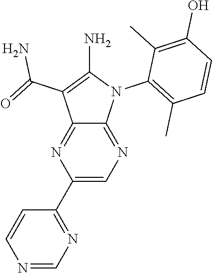 [SMILES: Cc1ccc(O)c(C)c1-n1c(N)c(C(N)=O)c2nc(-c3ccncn3)cnc21] | 57 |
| 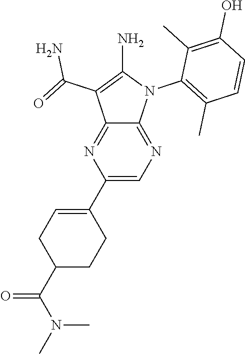 [SMILES: Cc1ccc(O)c(C)c1-n1c(N)c(C(N)=O)c2nc(C3=CCC(C(=O)N(C)C)CC3)cnc21] | 58 |
| 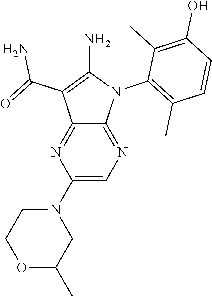 [SMILES: Cc1ccc(O)c(C)c1-n1c(N)c(C(N)=O)c2nc(N3CCOC(C)C3)cnc21] | 59 |
| 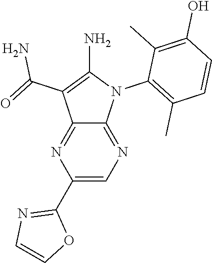 [SMILES: Cc1ccc(O)c(C)c1-n1c(N)c(C(N)=O)c2nc(-c3ncco3)cnc21] | 60 |
| 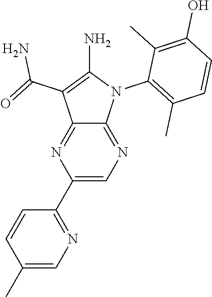 [SMILES: Cc1ccc(-c2cnc3c(n2)c(C(N)=O)c(N)n3-c2c(C)ccc(O)c2C)nc1] | 61 |
| 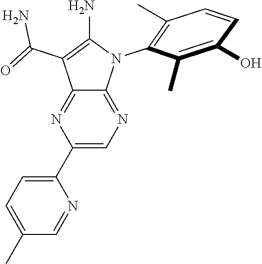 | 62 |
| 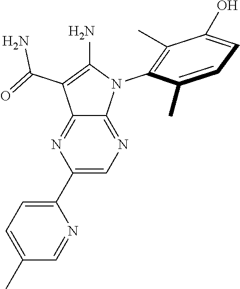 | 63 |
| 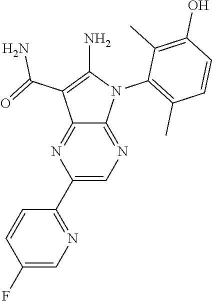 [SMILES: Cc1ccc(O)c(C)c1-n1c(N)c(C(N)=O)c2nc(-c3ccc(F)cn3)cnc21] | 64 |
| 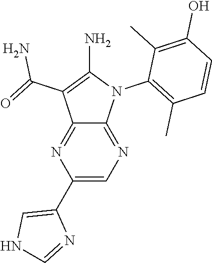 [SMILES: Cc1ccc(O)c(C)c1-n1c(N)c(C(N)=O)c2nc(-c3c[nH]cn3)cnc21] | 65 |
| 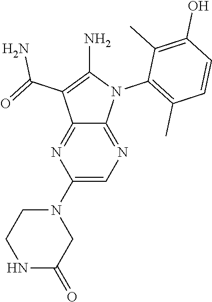 [SMILES: Cc1ccc(O)c(C)c1-n1c(N)c(C(N)=O)c2nc(N3CCNC(=O)C3)cnc21] | 66 |
| 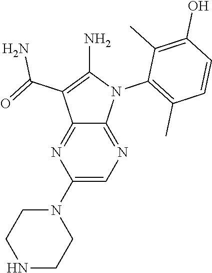 [SMILES: Cc1ccc(O)c(C)c1-n1c(N)c(C(N)=O)c2nc(N3CCNCC3)cnc21] | 67 |
| 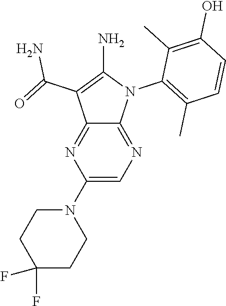 [SMILES: Cc1ccc(O)c(C)c1-n1c(N)c(C(N)=O)c2nc(N3CCC(F)(F)CC3)cnc21] | 68 |
| 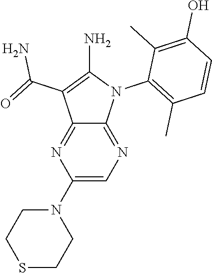 [SMILES: Cc1ccc(O)c(C)c1-n1c(N)c(C(N)=O)c2nc(N3CCSCC3)cnc21] | 69 |
| 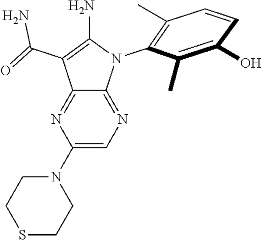 | 70 |
| 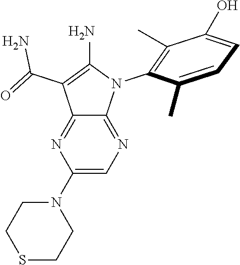 | 71 |
| 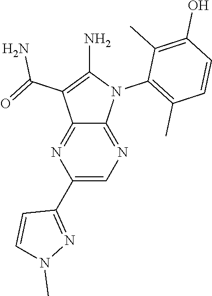 [SMILES: Cc1ccc(O)c(C)c1-n1c(N)c(C(N)=O)c2nc(-c3ccn(C)n3)cnc21] | 72 |
| 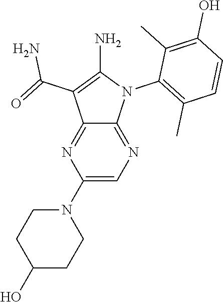 [SMILES: Cc1ccc(O)c(C)c1-n1c(N)c(C(N)=O)c2nc(N3CCC(O)CC3)cnc21] | 73 |
| 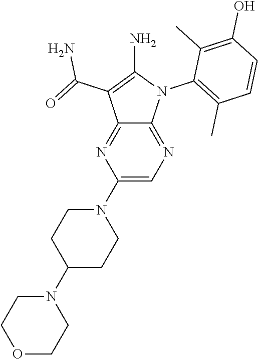 [SMILES: Cc1ccc(O)c(C)c1-n1c(N)c(C(N)=O)c2nc(N3CCC(N4CCOCC4)CC3)cnc21] | 74 |
| 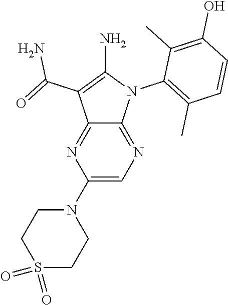 [SMILES: Cc1ccc(O)c(C)c1-n1c(N)c(C(N)=O)c2nc(N3CCS(=O)(=O)CC3)cnc21] | 75 |
| 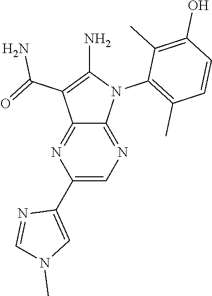 [SMILES: Cc1ccc(O)c(C)c1-n1c(N)c(C(N)=O)c2nc(-c3cn(C)cn3)cnc21] | 76 |
| 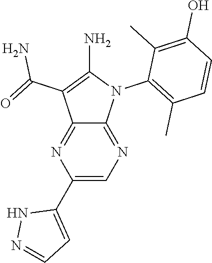 [SMILES: Cc1ccc(O)c(C)c1-n1c(N)c(C(N)=O)c2nc(-c3ccn[nH]3)cnc21] | 77 |
| 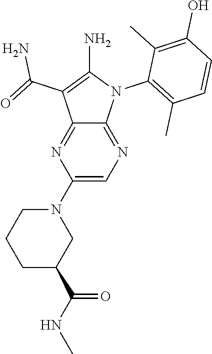 [SMILES: CNC(=O)[C@H]1CCCN(c2cnc3c(n2)c(C(N)=O)c(N)n3-c2c(C)ccc(O)c2C)C1] | 78 |
| 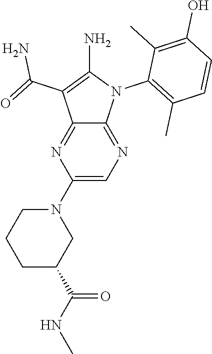 [SMILES: CNC(=O)[C@@H]1CCCN(c2cnc3c(n2)c(C(N)=O)c(N)n3-c2c(C)ccc(O)c2C)C1] | 79 |
| 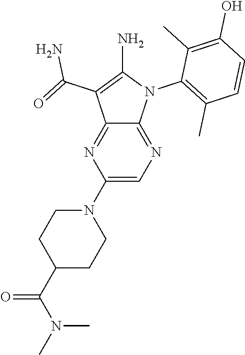 [SMILES: Cc1ccc(O)c(C)c1-n1c(N)c(C(N)=O)c2nc(N3CCC(C(=O)N(C)C)CC3)cnc21] | 80 |
| 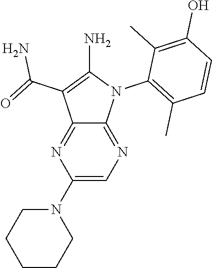 [SMILES: Cc1ccc(O)c(C)c1-n1c(N)c(C(N)=O)c2nc(N3CCCCC3)cnc21] | 81 |
| 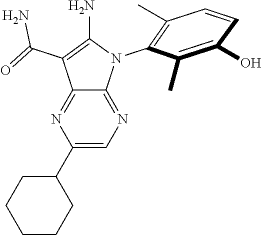 | 82 |
| 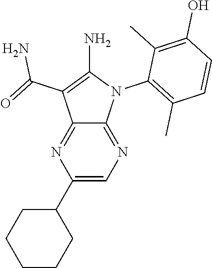 [SMILES: Cc1ccc(O)c(C)c1-n1c(N)c(C(N)=O)c2nc(C3CCCCC3)cnc21] | 83 |
| 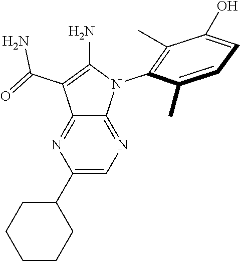 | 84 |
| 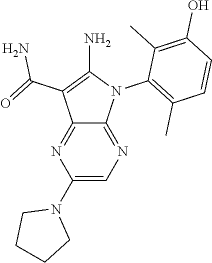 [SMILES: Cc1ccc(O)c(C)c1-n1c(N)c(C(N)=O)c2nc(N3CCCC3)cnc21] | 85 |
| 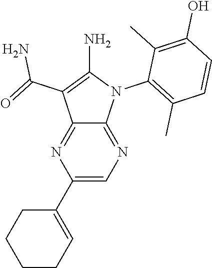 [SMILES: Cc1ccc(O)c(C)c1-n1c(N)c(C(N)=O)c2nc(C3=CCCCC3)cnc21] | 86 |
| 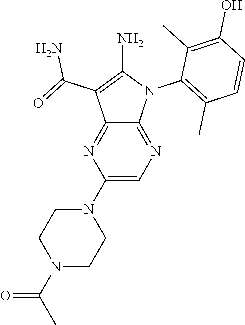 [SMILES: CC(=O)N1CCN(c2cnc3c(n2)c(C(N)=O)c(N)n3-c2c(C)ccc(O)c2C)CC1] | 87 |
| 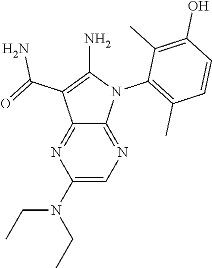 [SMILES: CCN(CC)c1cnc2c(n1)c(C(N)=O)c(N)n2-c1c(C)ccc(O)c1C] | 88 |
| 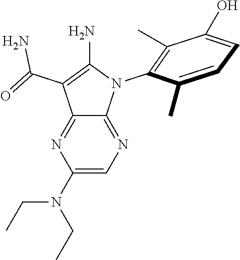 | 89 |
| 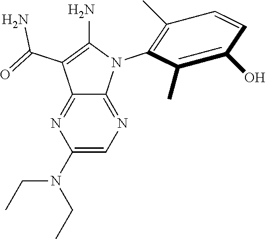 | 90 |
| 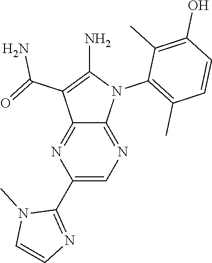 [SMILES: Cc1ccc(O)c(C)c1-n1c(N)c(C(N)=O)c2nc(-c3nccn3C)cnc21] | 91 |
| 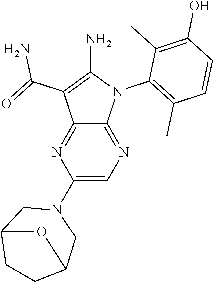 [SMILES: Cc1ccc(O)c(C)c1-n1c(N)c(C(N)=O)c2nc(N3CC4CCC(C3)O4)cnc21] | 92 |
| 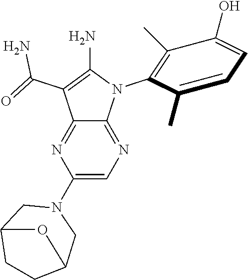 | 93 |
| 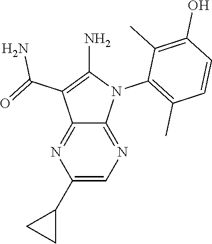 [SMILES: Cc1ccc(O)c(C)c1-n1c(N)c(C(N)=O)c2nc(C3CC3)cnc21] | 94 |
| 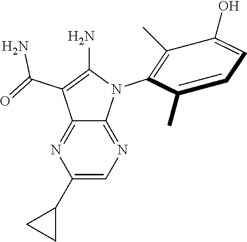 | 95 |
| 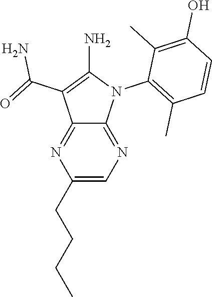 [SMILES: CCCCc1cnc2c(n1)c(C(N)=O)c(N)n2-c1c(C)ccc(O)c1C] | 96 |
| 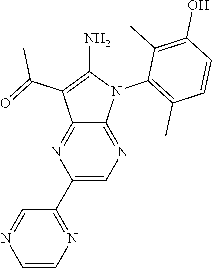 [SMILES: CC(=O)c1c(N)n(-c2c(C)ccc(O)c2C)c2ncc(-c3cnccn3)nc12] | 97 |
| 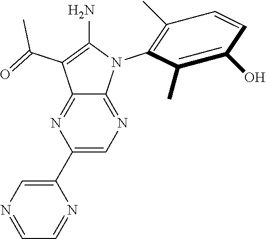 | 98 |
| 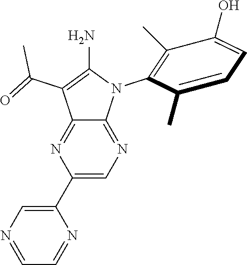 | 99 |
| 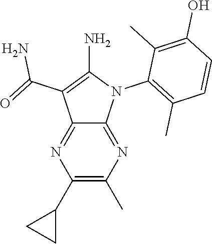 [SMILES: Cc1ccc(O)c(C)c1-n1c(N)c(C(N)=O)c2nc(C3CC3)c(C)nc21] | 100 |
| 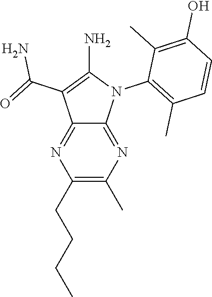 [SMILES: CCCCc1nc2c(C(N)=O)c(N)n(-c3c(C)ccc(O)c3C)c2nc1C] | 101 |
| 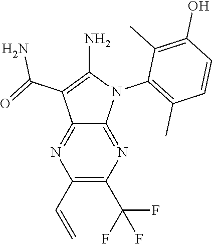 [SMILES: C=Cc1nc2c(C(N)=O)c(N)n(-c3c(C)ccc(O)c3C)c2nc1C(F)(F)F] | 102 |
| 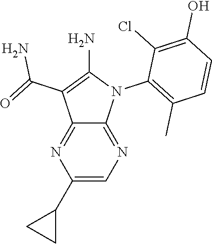 [SMILES: Cc1ccc(O)c(Cl)c1-n1c(N)c(C(N)=O)c2nc(C3CC3)cnc21] | 104 |
| 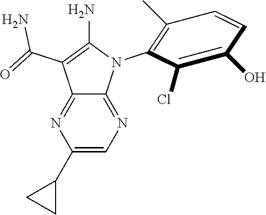 | 105 |
| 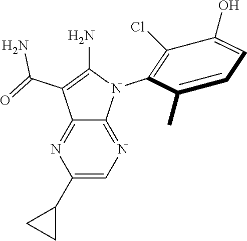 | 106 |
| 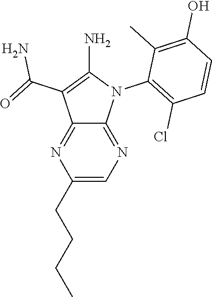 [SMILES: CCCCc1cnc2c(n1)c(C(N)=O)c(N)n2-c1c(Cl)ccc(O)c1C] | 107 |
| 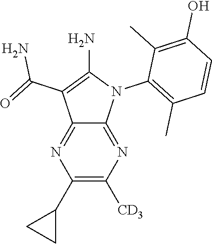 [SMILES: [2H]C([2H])([2H])c1nc2c(nc1C1CC1)c(C(N)=O)c(N)n2-c1c(C)ccc(O)c1C] | 110 |
| 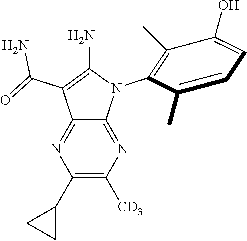 | 111 |
| 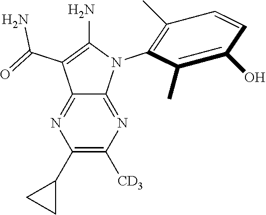 | 112 |
| 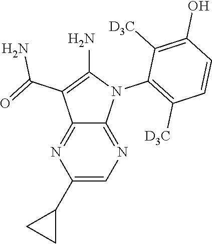 [SMILES: [2H]C([2H])([2H])c1ccc(O)c(C([2H])([2H])[2H])c1-n1c(N)c(C(N)=O)c2nc(C3CC3)cnc21] | 113 |
| 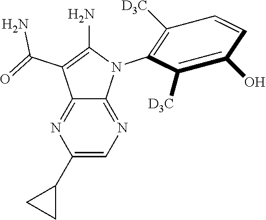 | 114 |
| 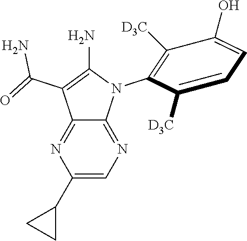 | 115 |
| 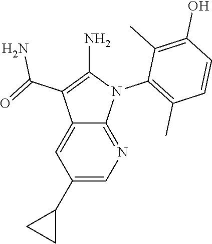 [SMILES: Cc1ccc(O)c(C)c1-n1c(N)c(C(N)=O)c2cc(C3CC3)cnc21] | 116 |
| 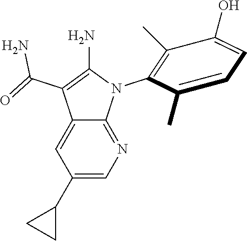 | 117 |
| 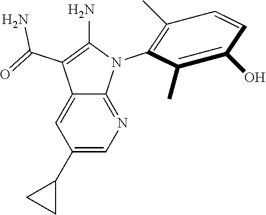 | 118 |
| 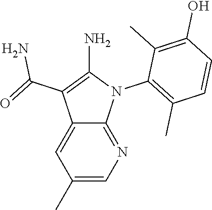 [SMILES: Cc1cnc2c(c1)c(C(N)=O)c(N)n2-c1c(C)ccc(O)c1C] | 119 |
| 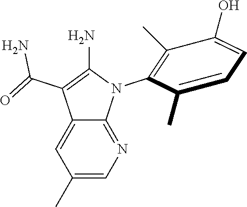 | 120 |
| 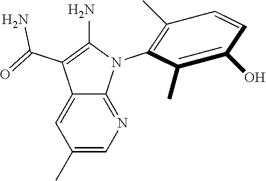 | 121 |
| 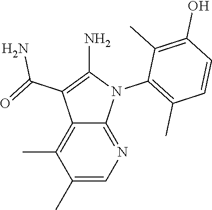 [SMILES: Cc1ccc(O)c(C)c1-n1c(N)c(C(N)=O)c2c(C)c(C)cnc21] | 125 |
| 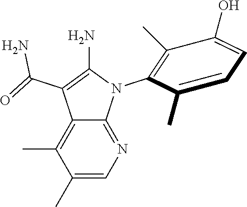 | 126 |
| 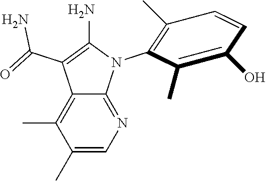 | 127 |
| 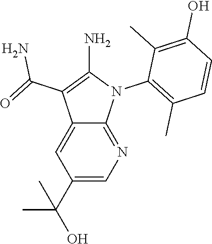 [SMILES: Cc1ccc(O)c(C)c1-n1c(N)c(C(N)=O)c2cc(C(C)(C)O)cnc21] | 131 |
| 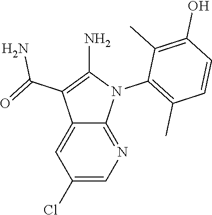 [SMILES: Cc1ccc(O)c(C)c1-n1c(N)c(C(N)=O)c2cc(Cl)cnc21] | 132 |
| 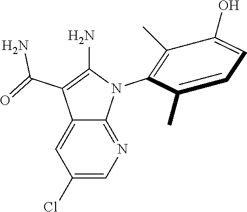 | 133 |
| 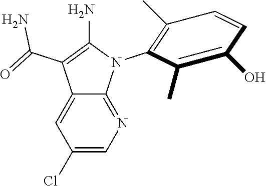 | 134 |
| 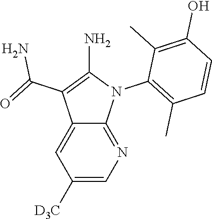 [SMILES: [2H]C([2H])([2H])c1cnc2c(c1)c(C(N)=O)c(N)n2-c1c(C)ccc(O)c1C] | 135 |
| 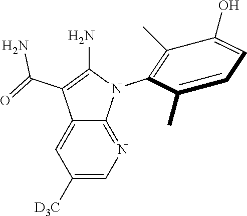 | 136 |
| 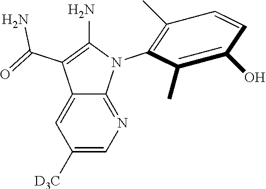 | 137 |
| 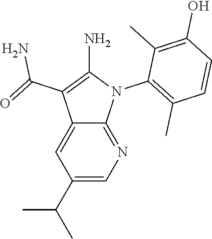 [SMILES: Cc1ccc(O)c(C)c1-n1c(N)c(C(N)=O)c2cc(C(C)C)cnc21] | 138 |
| 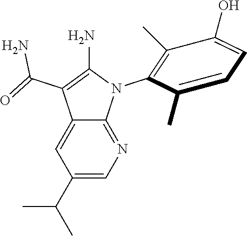 | 139 |
| 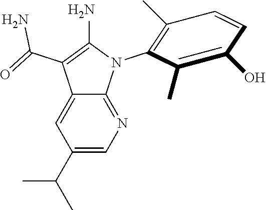 | 140 |
| 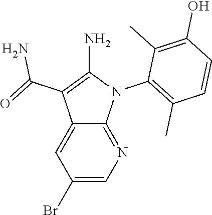 [SMILES: Cc1ccc(O)c(C)c1-n1c(N)c(C(N)=O)c2cc(Br)cnc21] | 141 |
| 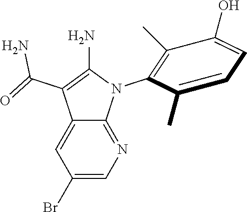 | 142 |
| 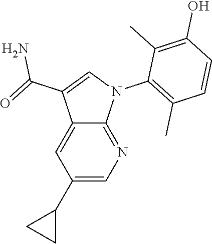 [SMILES: Cc1ccc(O)c(C)c1-n1cc(C(N)=O)c2cc(C3CC3)cnc21] | 143 |
| 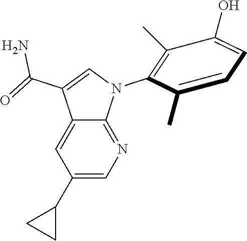 | 144 |
| 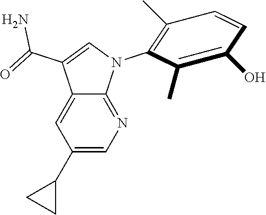 | 145 |
| 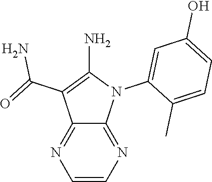 [SMILES: Cc1ccc(O)cc1-n1c(N)c(C(N)=O)c2nccnc21] | 146 |
| 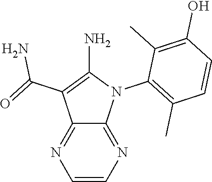 [SMILES: Cc1ccc(O)c(C)c1-n1c(N)c(C(N)=O)c2nccnc21] | 147 |
| 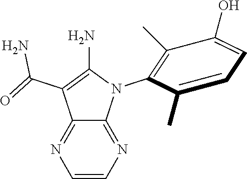 | 148 |
| 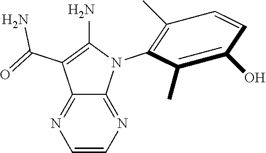 | 149 |
| 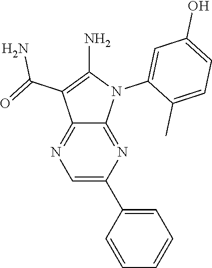 [SMILES: Cc1ccc(O)cc1-n1c(N)c(C(N)=O)c2ncc(-c3ccccc3)nc21] | 150 |
| 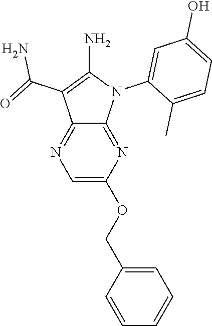 [SMILES: Cc1ccc(O)cc1-n1c(N)c(C(N)=O)c2ncc(OCc3ccccc3)nc21] | 151 |
| 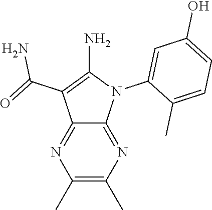 [SMILES: Cc1ccc(O)cc1-n1c(N)c(C(N)=O)c2nc(C)c(C)nc21] | 152 |
| 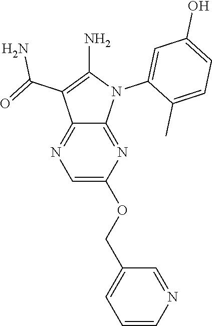 [SMILES: Cc1ccc(O)cc1-n1c(N)c(C(N)=O)c2ncc(OCc3cccnc3)nc21] | 153 |
| 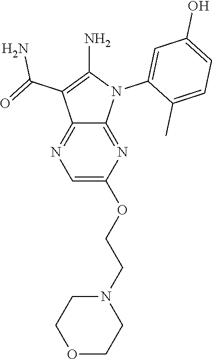 [SMILES: Cc1ccc(O)cc1-n1c(N)c(C(N)=O)c2ncc(OCCN3CCOCC3)nc21] | 154 |
| 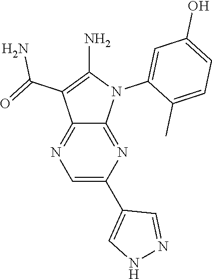 [SMILES: Cc1ccc(O)cc1-n1c(N)c(C(N)=O)c2ncc(-c3cn[nH]c3)nc21] | 155 |
| 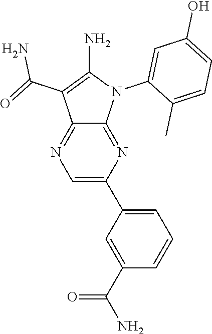 [SMILES: Cc1ccc(O)cc1-n1c(N)c(C(N)=O)c2ncc(-c3cccc(C(N)=O)c3)nc21] | 156 |
| 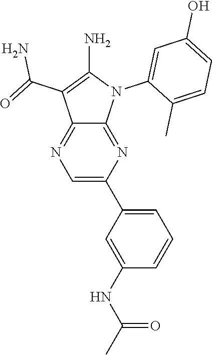 [SMILES: CC(=O)Nc1cccc(-c2cnc3c(C(N)=O)c(N)n(-c4cc(O)ccc4C)c3n2)c1] | 157 |
| 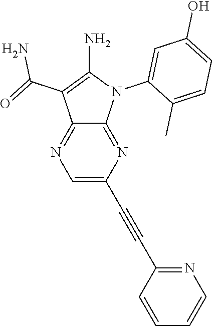 [SMILES: Cc1ccc(O)cc1-n1c(N)c(C(N)=O)c2ncc(C#Cc3ccccn3)nc21] | 158 |
| 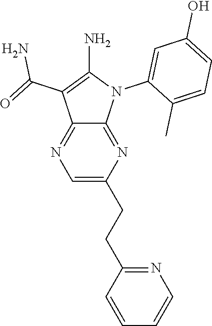 [SMILES: Cc1ccc(O)cc1-n1c(N)c(C(N)=O)c2ncc(CCc3ccccn3)nc21] | 159 |
| 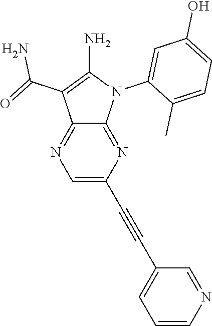 [SMILES: Cc1ccc(O)cc1-n1c(N)c(C(N)=O)c2ncc(C#Cc3cccnc3)nc21] | 160 |
| 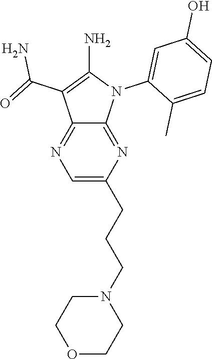 [SMILES: Cc1ccc(O)cc1-n1c(N)c(C(N)=O)c2ncc(CCCN3CCOCC3)nc21] | 161 |
| 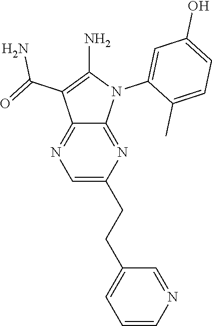 [SMILES: Cc1ccc(O)cc1-n1c(N)c(C(N)=O)c2ncc(CCc3cccnc3)nc21] | 162 |
| 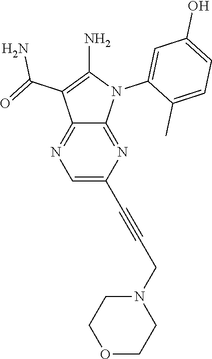 [SMILES: Cc1ccc(O)cc1-n1c(N)c(C(N)=O)c2ncc(C#CCN3CCOCC3)nc21] | 163 |
| 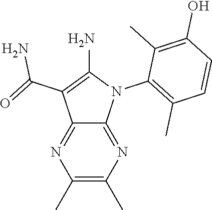 [SMILES: Cc1ccc(O)c(C)c1-n1c(N)c(C(N)=O)c2nc(C)c(C)nc21] | 164 |
| 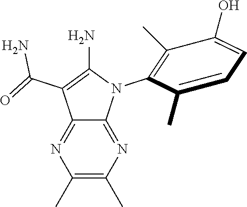 | 165 |
| 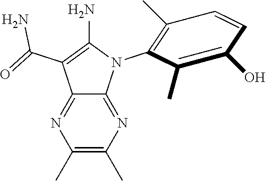 | 166 |
| 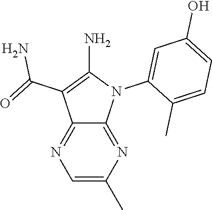 [SMILES: Cc1cnc2c(C(N)=O)c(N)n(-c3cc(O)ccc3C)c2n1] | 167 |
| 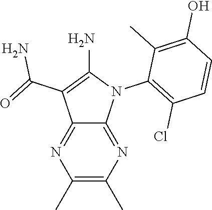 [SMILES: Cc1nc2c(C(N)=O)c(N)n(-c3c(Cl)ccc(O)c3C)c2nc1C] | 168 |
| 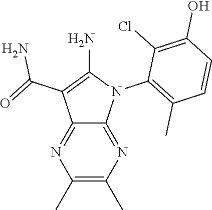 [SMILES: Cc1ccc(O)c(Cl)c1-n1c(N)c(C(N)=O)c2nc(C)c(C)nc21] | 170 |
| 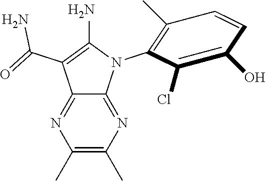 | 171 |
| 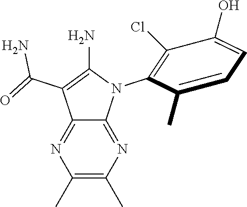 | 172 |
| 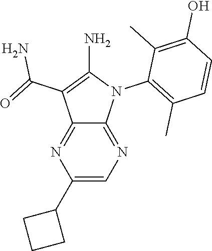 [SMILES: Cc1ccc(O)c(C)c1-n1c(N)c(C(N)=O)c2nc(C3CCC3)cnc21] | 173 |
| 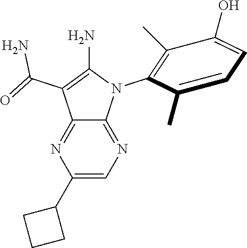 | 174 |
| 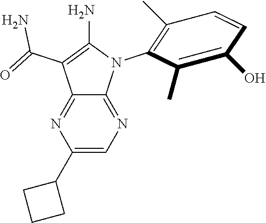 | 175 |
| 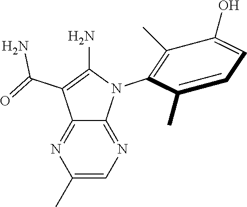 | 176 |
| 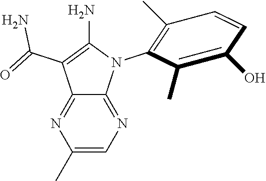 | 177 |
| 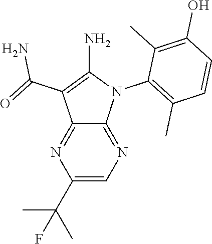 [SMILES: Cc1ccc(O)c(C)c1-n1c(N)c(C(N)=O)c2nc(C(C)(C)F)cnc21] | 178 |
| 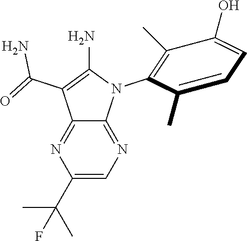 | 179 |
| 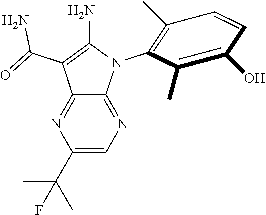 | 180 |
| 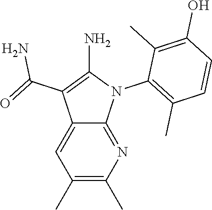 [SMILES: Cc1cc2c(C(N)=O)c(N)n(-c3c(C)ccc(O)c3C)c2nc1C] | 181 |
| 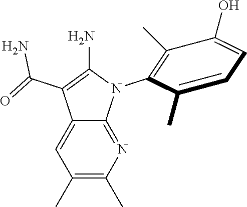 | 182 |
| 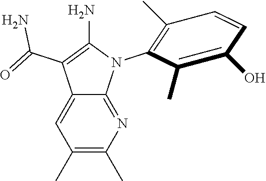 | 183 |
| 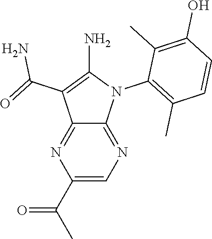 [SMILES: CC(=O)c1cnc2c(n1)c(C(N)=O)c(N)n2-c1c(C)ccc(O)c1C] | 184 |
| 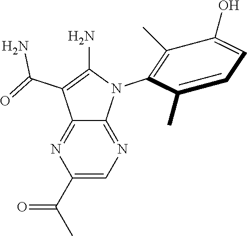 | 185 |
| 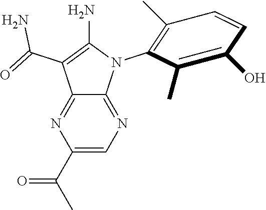 | 186 |
| 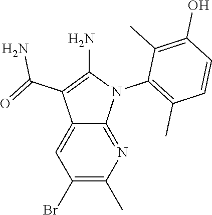 [SMILES: Cc1ccc(O)c(C)c1-n1c(N)c(C(N)=O)c2cc(Br)c(C)nc21] | 187 |
| 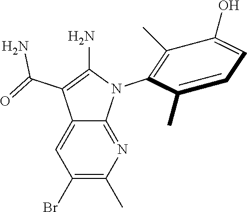 | 188 |
| 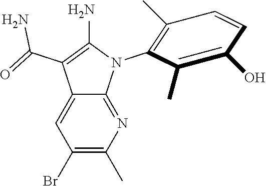 | 189 |
| 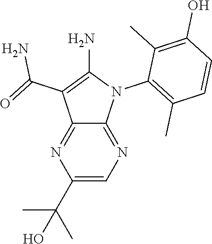 [SMILES: Cc1ccc(O)c(C)c1-n1c(N)c(C(N)=O)c2nc(C(C)(C)O)cnc21] | 190 |
| 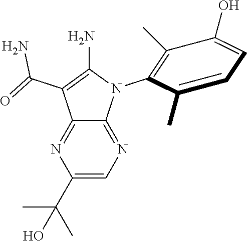 | 191 |
| 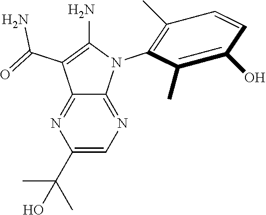 | 192 |
| 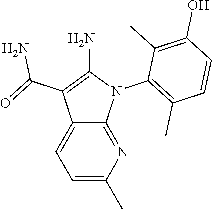 [SMILES: Cc1ccc2c(C(N)=O)c(N)n(-c3c(C)ccc(O)c3C)c2n1] | 193 |
| 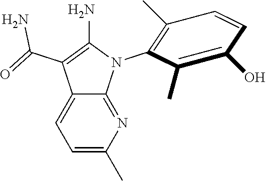 | 194 |
| 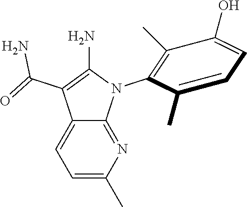 | 195 |
| 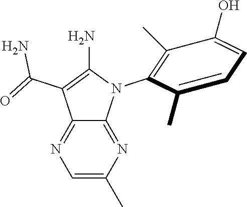 | 196 |
| 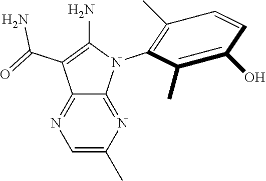 | 197 |
| 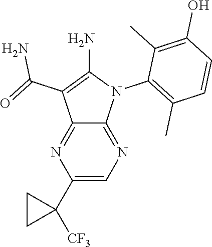 [SMILES: Cc1ccc(O)c(C)c1-n1c(N)c(C(N)=O)c2nc(C3(C(F)(F)F)CC3)cnc21] | 198 |
| 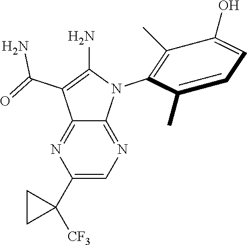 | 199 |
| 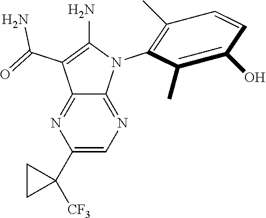 | 200 |
| 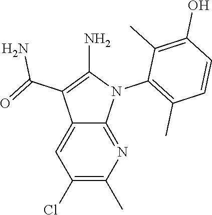 [SMILES: Cc1ccc(O)c(C)c1-n1c(N)c(C(N)=O)c2cc(Cl)c(C)nc21] | 201 |
| 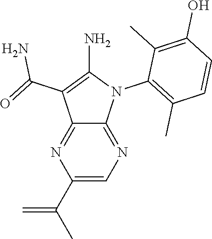 [SMILES: C=C(C)c1cnc2c(n1)c(C(N)=O)c(N)n2-c1c(C)ccc(O)c1C] | 202 |
| 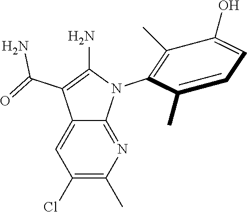 | 203 |
| 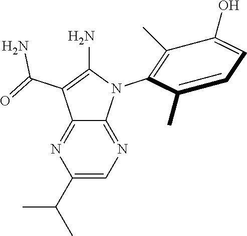 | 204 |
| 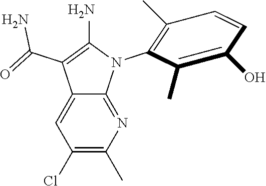 | 205 |
| 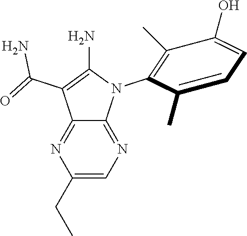 | 206 |
| 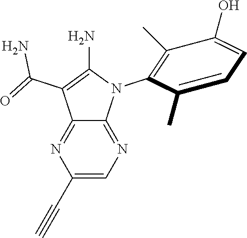 | 208 |
| 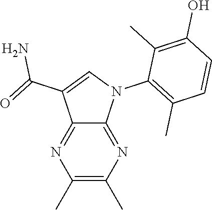 [SMILES: Cc1ccc(O)c(C)c1-n1cc(C(N)=O)c2nc(C)c(C)nc21] | 209 |
| 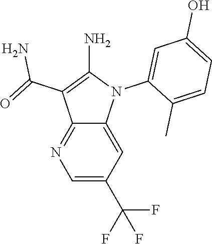 [SMILES: Cc1ccc(O)cc1-n1c(N)c(C(N)=O)c2ncc(C(F)(F)F)cc21] | 212 |
| 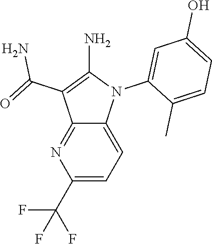 [SMILES: Cc1ccc(O)cc1-n1c(N)c(C(N)=O)c2nc(C(F)(F)F)ccc21] | 213 |
| 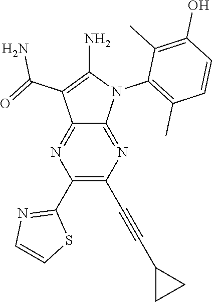 [SMILES: Cc1ccc(O)c(C)c1-n1c(N)c(C(N)=O)c2nc(-c3nccs3)c(C#CC3CC3)nc21] | 214 |
| 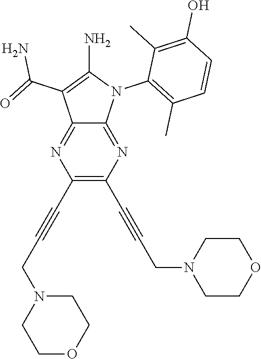 [SMILES: Cc1ccc(O)c(C)c1-n1c(N)c(C(N)=O)c2nc(C#CCN3CCOCC3)c(C#CCN3CCOCC3)nc21] | 215 |
| 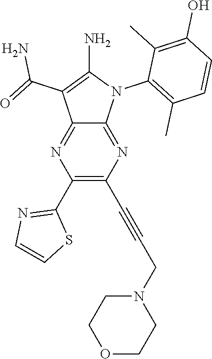 [SMILES: Cc1ccc(O)c(C)c1-n1c(N)c(C(N)=O)c2nc(-c3nccs3)c(C#CCN3CCOCC3)nc21] | 216 |
| 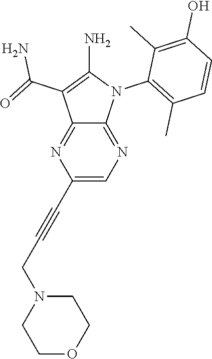 [SMILES: Cc1ccc(O)c(C)c1-n1c(N)c(C(N)=O)c2nc(C#CCN3CCOCC3)cnc21] | 217 |
| 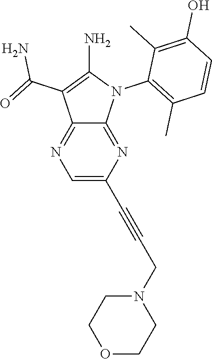 [SMILES: Cc1ccc(O)c(C)c1-n1c(N)c(C(N)=O)c2ncc(C#CCN3CCOCC3)nc21] | 218 |
| 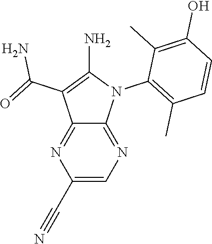 [SMILES: Cc1ccc(O)c(C)c1-n1c(N)c(C(N)=O)c2nc(C#N)cnc21] | 219 |
| 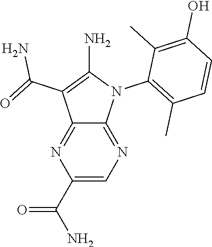 [SMILES: Cc1ccc(O)c(C)c1-n1c(N)c(C(N)=O)c2nc(C(N)=O)cnc21] | 220 |
| 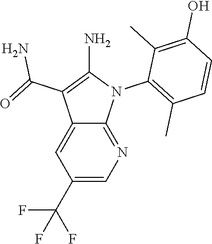 [SMILES: Cc1ccc(O)c(C)c1-n1c(N)c(C(N)=O)c2cc(C(F)(F)F)cnc21] | 221 |
| 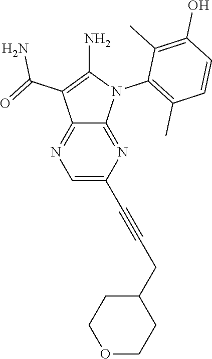 [SMILES: Cc1ccc(O)c(C)c1-n1c(N)c(C(N)=O)c2ncc(C#CCC3CCOCC3)nc21] | 222 |
| 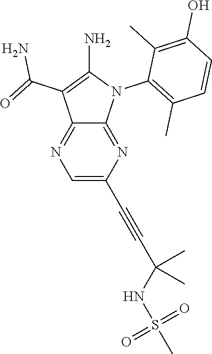 [SMILES: Cc1ccc(O)c(C)c1-n1c(N)c(C(N)=O)c2ncc(C#CC(C)(C)NS(C)(=O)=O)nc21] | 223 |
| 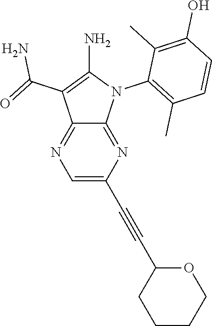 [SMILES: Cc1ccc(O)c(C)c1-n1c(N)c(C(N)=O)c2ncc(C#CC3CCCCO3)nc21] | 224 |
| 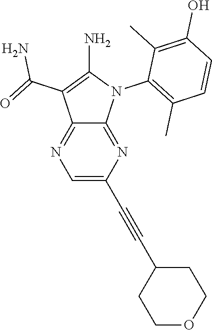 [SMILES: Cc1ccc(O)c(C)c1-n1c(N)c(C(N)=O)c2ncc(C#CC3CCOCC3)nc21] | 225 |
| 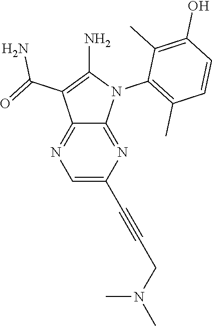 [SMILES: Cc1ccc(O)c(C)c1-n1c(N)c(C(N)=O)c2ncc(C#CCN(C)C)nc21] | 226 |
| 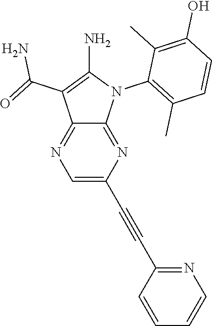 [SMILES: Cc1ccc(O)c(C)c1-n1c(N)c(C(N)=O)c2ncc(C#Cc3ccccn3)nc21] | 227 |
| 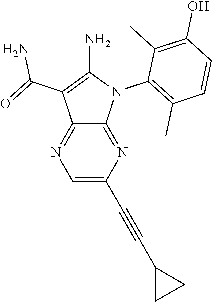 [SMILES: Cc1ccc(O)c(C)c1-n1c(N)c(C(N)=O)c2ncc(C#CC3CC3)nc21] | 228 |
| 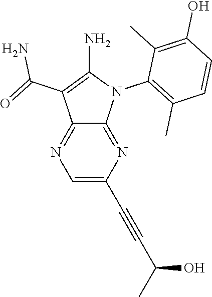 [SMILES: Cc1ccc(O)c(C)c1-n1c(N)c(C(N)=O)c2ncc(C#C[C@H](C)O)nc21] | 229 |
| 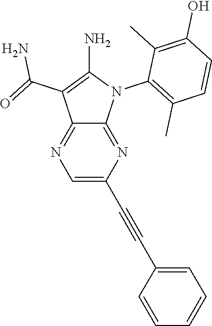 [SMILES: Cc1ccc(O)c(C)c1-n1c(N)c(C(N)=O)c2ncc(C#Cc3ccccc3)nc21] | 230 |
| 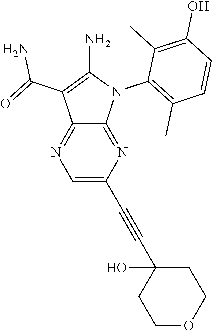 [SMILES: Cc1ccc(O)c(C)c1-n1c(N)c(C(N)=O)c2ncc(C#CC3(O)CCOCC3)nc21] | 231 |
| 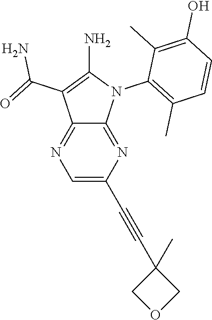 [SMILES: Cc1ccc(O)c(C)c1-n1c(N)c(C(N)=O)c2ncc(C#CC3(C)COC3)nc21] | 232 |
| 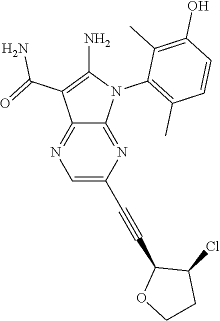 [SMILES: Cc1ccc(O)c(C)c1-n1c(N)c(C(N)=O)c2ncc(C#C[C@@H]3OCC[C@@H]3Cl)nc21] | 233 |
| 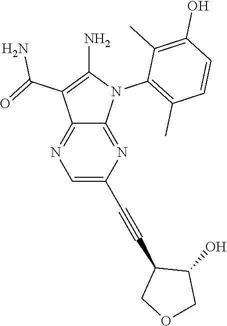 [SMILES: Cc1ccc(O)c(C)c1-n1c(N)c(C(N)=O)c2ncc(C#C[C@@H]3COC[C@H]3O)nc21] | 234 |
| 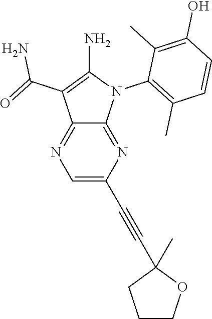 [SMILES: Cc1ccc(O)c(C)c1-n1c(N)c(C(N)=O)c2ncc(C#CC3(C)CCCO3)nc21] | 235 |
| 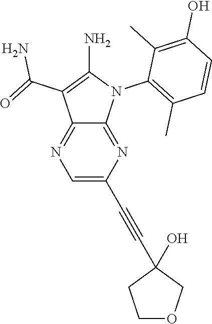 [SMILES: Cc1ccc(O)c(C)c1-n1c(N)c(C(N)=O)c2ncc(C#CC3(O)CCOC3)nc21] | 236 |
| 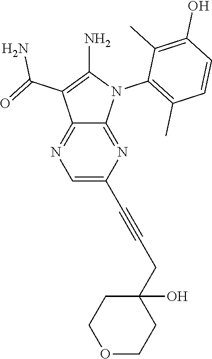 [SMILES: Cc1ccc(O)c(C)c1-n1c(N)c(C(N)=O)c2ncc(C#CCC3(O)CCOCC3)nc21] | 237 |
| 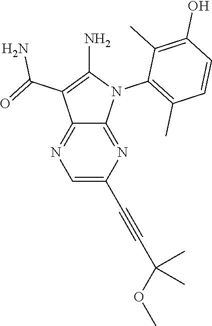 [SMILES: COC(C)(C)C#Cc1cnc2c(C(N)=O)c(N)n(-c3c(C)ccc(O)c3C)c2n1] | 238 |
| 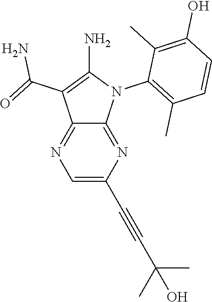 [SMILES: Cc1ccc(O)c(C)c1-n1c(N)c(C(N)=O)c2ncc(C#CC(C)(C)O)nc21] | 239 |
| 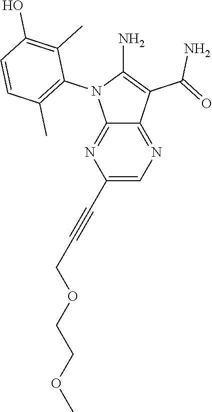 [SMILES: COCCOCC#Cc1cnc2c(C(N)=O)c(N)n(-c3c(C)ccc(O)c3C)c2n1] | 240 |
| 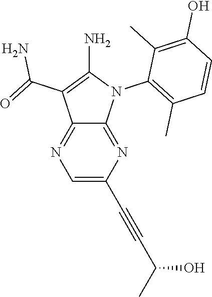 [SMILES: Cc1ccc(O)c(C)c1-n1c(N)c(C(N)=O)c2ncc(C#C[C@@H](C)O)nc21] | 241 |
| 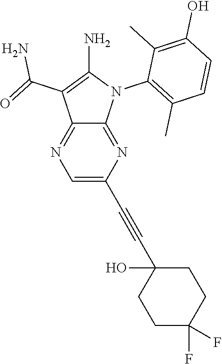 [SMILES: Cc1ccc(O)c(C)c1-n1c(N)c(C(N)=O)c2ncc(C#CC3(O)CCC(F)(F)CC3)nc21] | 242 |
| 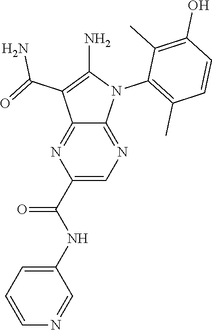 [SMILES: Cc1ccc(O)c(C)c1-n1c(N)c(C(N)=O)c2nc(C(=O)Nc3cccnc3)cnc21] | 243 |
| 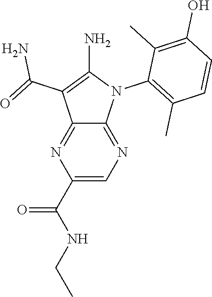 [SMILES: CCNC(=O)c1cnc2c(n1)c(C(N)=O)c(N)n2-c1c(C)ccc(O)c1C] | 244 |
| 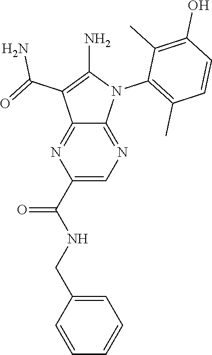 [SMILES: Cc1ccc(O)c(C)c1-n1c(N)c(C(N)=O)c2nc(C(=O)NCc3ccccc3)cnc21] | 245 |
| 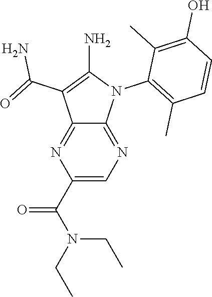 [SMILES: CCN(CC)C(=O)c1cnc2c(n1)c(C(N)=O)c(N)n2-c1c(C)ccc(O)c1C] | 246 |
| 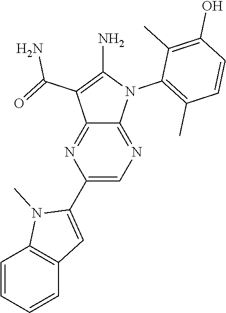 [SMILES: Cc1ccc(O)c(C)c1-n1c(N)c(C(N)=O)c2nc(-c3cc4ccccc4n3C)cnc21] | 247 |
| 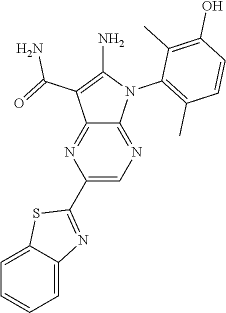 [SMILES: Cc1ccc(O)c(C)c1-n1c(N)c(C(N)=O)c2nc(-c3nc4ccccc4s3)cnc21] | 248 |
| 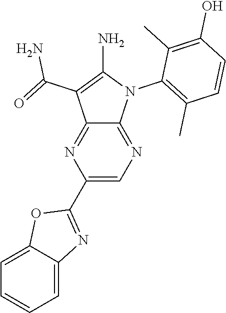 [SMILES: Cc1ccc(O)c(C)c1-n1c(N)c(C(N)=O)c2nc(-c3nc4ccccc4o3)cnc21] | 249 |
| 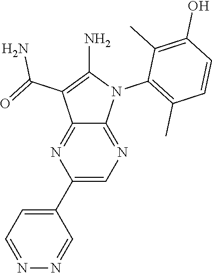 [SMILES: Cc1ccc(O)c(C)c1-n1c(N)c(C(N)=O)c2nc(-c3ccnnc3)cnc21] | 250 |
| 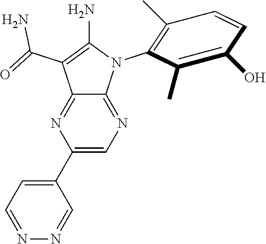 | 251 |
| 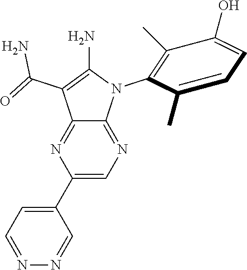 | 252 |
| 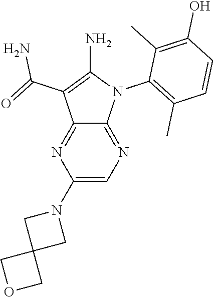 [SMILES: Cc1ccc(O)c(C)c1-n1c(N)c(C(N)=O)c2nc(N3CC4(COC4)C3)cnc21] | 253 |
| 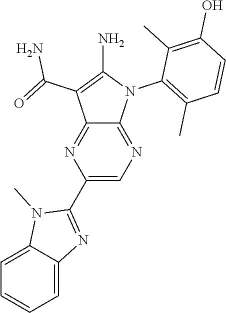 [SMILES: Cc1ccc(O)c(C)c1-n1c(N)c(C(N)=O)c2nc(-c3nc4ccccc4n3C)cnc21] | 254 |
| 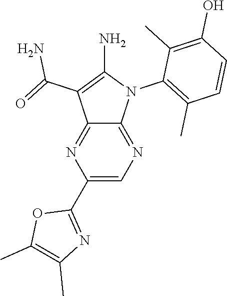 [SMILES: Cc1ccc(O)c(C)c1-n1c(N)c(C(N)=O)c2nc(-c3nc(C)c(C)o3)cnc21] | 255 |
| 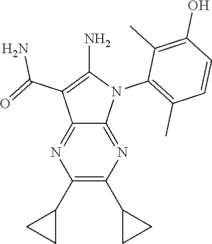 [SMILES: Cc1ccc(O)c(C)c1-n1c(N)c(C(N)=O)c2nc(C3CC3)c(C3CC3)nc21] | 256 |
| 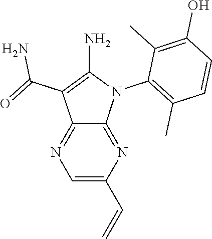 [SMILES: C=Cc1cnc2c(C(N)=O)c(N)n(-c3c(C)ccc(O)c3C)c2n1] | 257 |
| 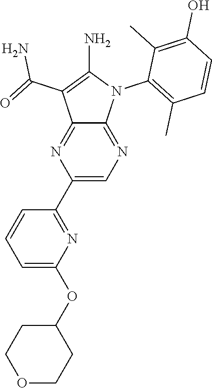 [SMILES: Cc1ccc(O)c(C)c1-n1c(N)c(C(N)=O)c2nc(-c3cccc(OC4CCOCC4)n3)cnc21] | 258 |
| 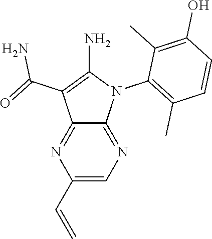 [SMILES: C=Cc1cnc2c(n1)c(C(N)=O)c(N)n2-c1c(C)ccc(O)c1C] | 259 |
| 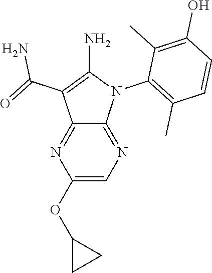 [SMILES: Cc1ccc(O)c(C)c1-n1c(N)c(C(N)=O)c2nc(OC3CC3)cnc21] | 260 |
| 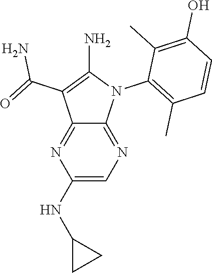 [SMILES: Cc1ccc(O)c(C)c1-n1c(N)c(C(N)=O)c2nc(NC3CC3)cnc21] | 261 |
| 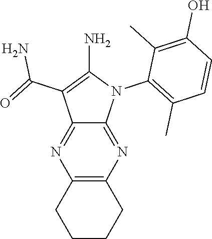 [SMILES: Cc1ccc(O)c(C)c1-n1c(N)c(C(N)=O)c2nc3c(nc21)CCCC3] | 262 |
| 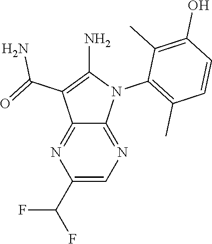 [SMILES: Cc1ccc(O)c(C)c1-n1c(N)c(C(N)=O)c2nc(C(F)F)cnc21] | 263 |
| 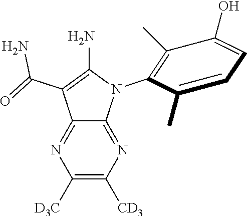 | 264 |
| 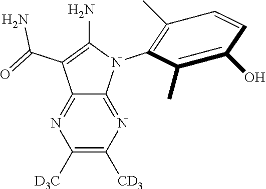 | 265 |
| 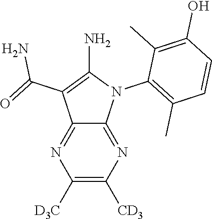 [SMILES: [2H]C([2H])([2H])c1nc2c(C(N)=O)c(N)n(-c3c(C)ccc(O)c3C)c2nc1C([2H])([2H])[2H]] | 266 |
| 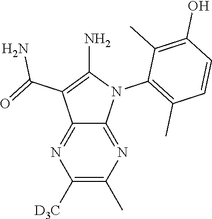 [SMILES: [2H]C([2H])([2H])c1nc2c(C(N)=O)c(N)n(-c3c(C)ccc(O)c3C)c2nc1C] | 267 |
| 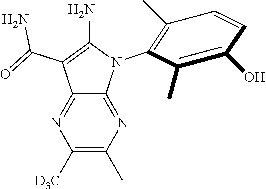 | 268 |
| 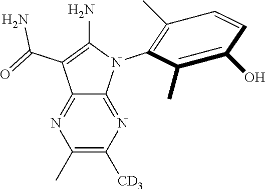 | 269 |
| 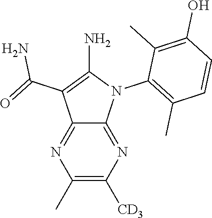 [SMILES: [2H]C([2H])([2H])c1nc2c(nc1C)c(C(N)=O)c(N)n2-c1c(C)ccc(O)c1C] | 270 |
| 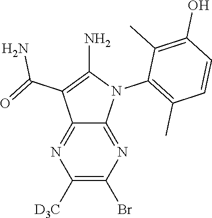 [SMILES: [2H]C([2H])([2H])c1nc2c(C(N)=O)c(N)n(-c3c(C)ccc(O)c3C)c2nc1Br] | 271 |
| 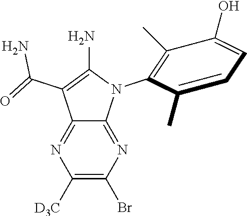 | 272 |
| 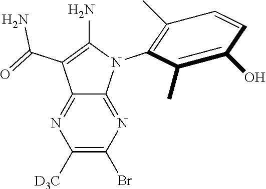 | 273 |
| 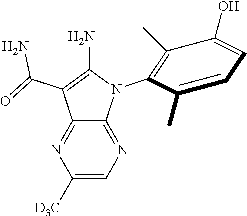 | 274 |
| 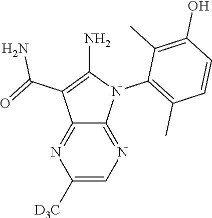 [SMILES: [2H]C([2H])([2H])c1cnc2c(n1)c(C(N)=O)c(N)n2-c1c(C)ccc(O)c1C] | 275 |
| 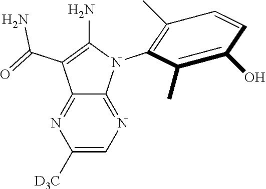 | 276 |
| 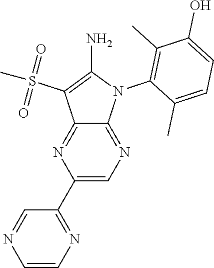 [SMILES: Cc1ccc(O)c(C)c1-n1c(N)c(S(C)(=O)=O)c2nc(-c3cnccn3)cnc21] | 278 |
| 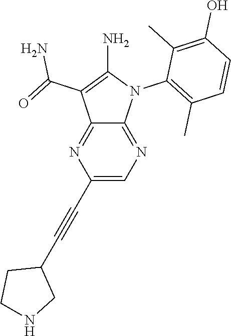 [SMILES: Cc1ccc(O)c(C)c1-n1c(N)c(C(N)=O)c2nc(C#CC3CCNC3)cnc21] | 279 |
| 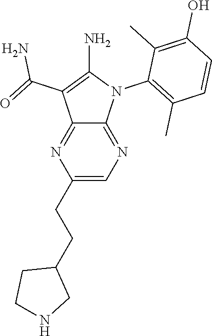 [SMILES: Cc1ccc(O)c(C)c1-n1c(N)c(C(N)=O)c2nc(CCC3CCNC3)cnc21] | 280 |
| 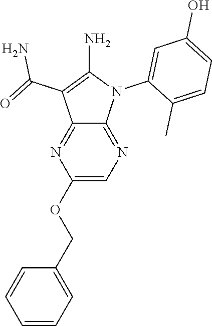 [SMILES: Cc1ccc(O)cc1-n1c(N)c(C(N)=O)c2nc(OCc3ccccc3)cnc21] | 281 |
| 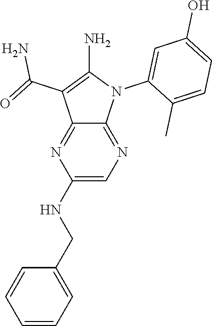 [SMILES: Cc1ccc(O)cc1-n1c(N)c(C(N)=O)c2nc(NCc3ccccc3)cnc21] | 282 |
| 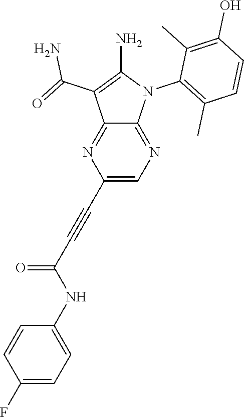 [SMILES: Cc1ccc(O)c(C)c1-n1c(N)c(C(N)=O)c2nc(C#CC(=O)Nc3ccc(F)cc3)cnc21] | 283 |
| 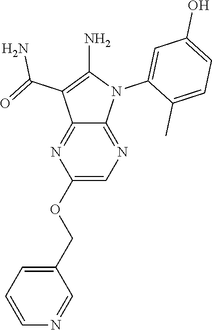 [SMILES: Cc1ccc(O)cc1-n1c(N)c(C(N)=O)c2nc(OCc3cccnc3)cnc21] | 284 |
| 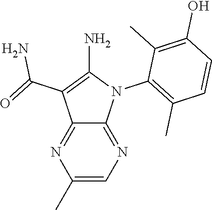 [SMILES: Cc1cnc2c(n1)c(C(N)=O)c(N)n2-c1c(C)ccc(O)c1C] | 285 |
| 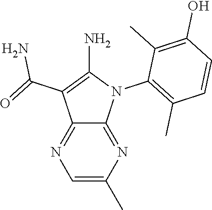 [SMILES: Cc1cnc2c(C(N)=O)c(N)n(-c3c(C)ccc(O)c3C)c2n1] | 286 |
| 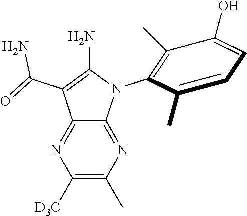 | 287 |
| 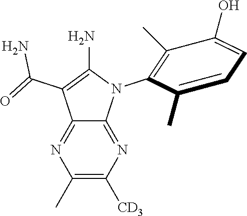 | 288 |
| 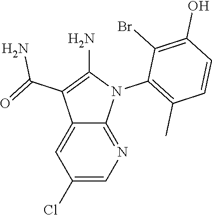 [SMILES: Cc1ccc(O)c(Br)c1-n1c(N)c(C(N)=O)c2cc(Cl)cnc21] | 289 |
| 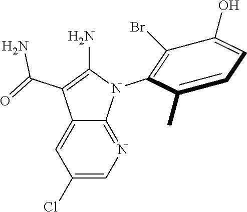 | 290 |
| 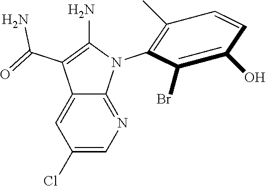 | 291 |
| 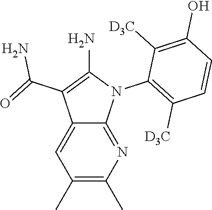 [SMILES: [2H]C([2H])([2H])c1ccc(O)c(C([2H])([2H])[2H])c1-n1c(N)c(C(N)=O)c2cc(C)c(C)nc21] | 292 |
| 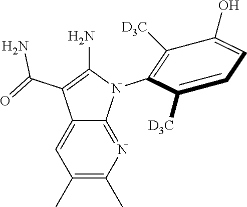 | 293 |
| 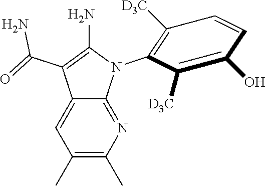 | 294 |
| 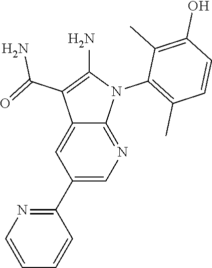 [SMILES: Cc1ccc(O)c(C)c1-n1c(N)c(C(N)=O)c2cc(-c3ccccn3)cnc21] | 295 |
| 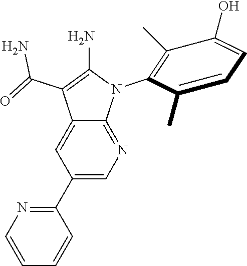 | 296 |
| 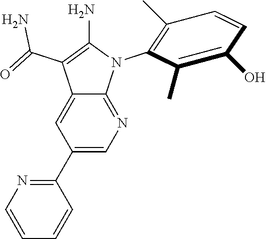 | 297 |
| 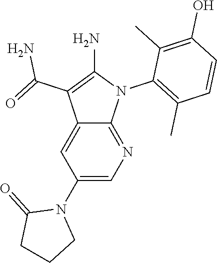 [SMILES: Cc1ccc(O)c(C)c1-n1c(N)c(C(N)=O)c2cc(N3CCCC3=O)cnc21] | 298 |
| 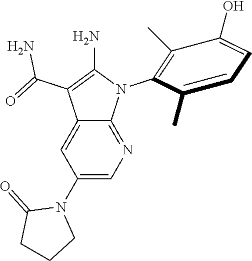 | 299 |
| 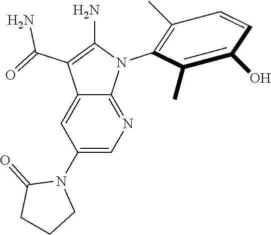 | 300 |
| 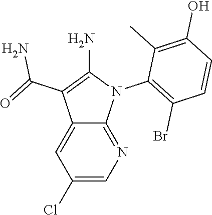 [SMILES: Cc1c(O)ccc(Br)c1-n1c(N)c(C(N)=O)c2cc(Cl)cnc21] | 301 |
| 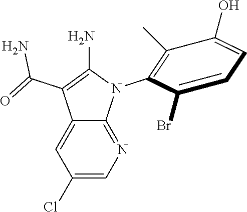 | 302 |
| 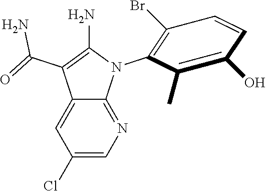 | 303 |
| 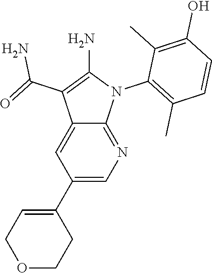 [SMILES: Cc1ccc(O)c(C)c1-n1c(N)c(C(N)=O)c2cc(C3=CCOCC3)cnc21] | 304 |
| 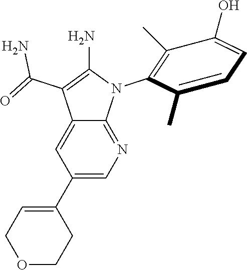 | 305 |
| 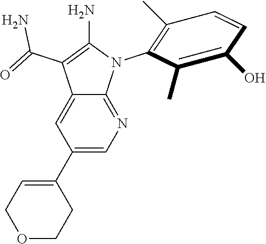 | 306 |
| 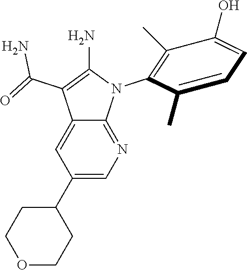 | 307 |
| 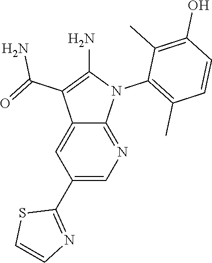 [SMILES: Cc1ccc(O)c(C)c1-n1c(N)c(C(N)=O)c2cc(-c3nccs3)cnc21] | 308 |
| 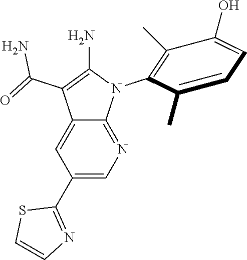 | 309 |
| 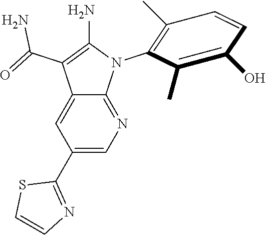 | 310 |
| 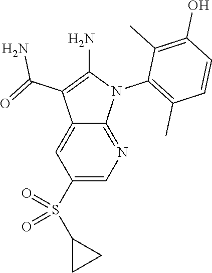 [SMILES: Cc1ccc(O)c(C)c1-n1c(N)c(C(N)=O)c2cc(S(=O)(=O)C3CC3)cnc21] | 311 |
| 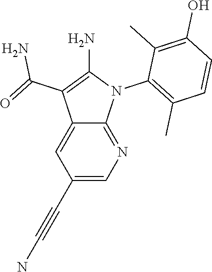 [SMILES: Cc1ccc(O)c(C)c1-n1c(N)c(C(N)=O)c2cc(C#CN)cnc21] | 312 |
| 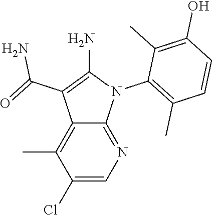 [SMILES: Cc1ccc(O)c(C)c1-n1c(N)c(C(N)=O)c2c(C)c(Cl)cnc21] | 313 |
| 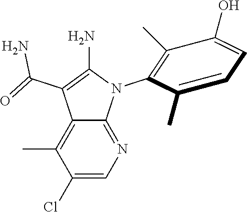 | 314 |
| 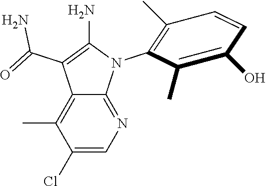 | 315 |
| 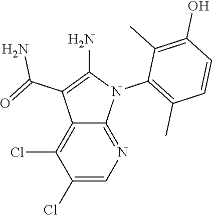 [SMILES: Cc1ccc(O)c(C)c1-n1c(N)c(C(N)=O)c2c(Cl)c(Cl)cnc21] | 316 |
| 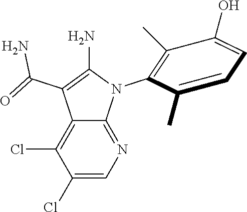 | 317 |
| 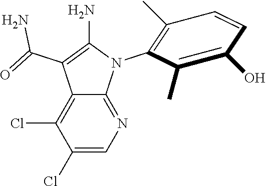 | 318 |
| 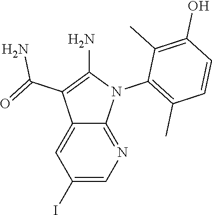 [SMILES: Cc1ccc(O)c(C)c1-n1c(N)c(C(N)=O)c2cc(I)cnc21] | 319 |
| 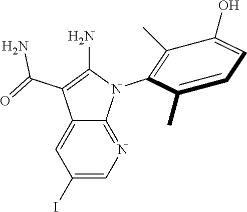 | 320 |
| 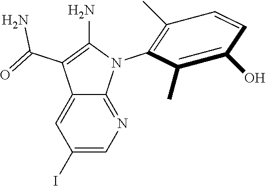 | 321 |
| 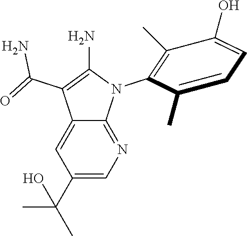 | 322 |
| 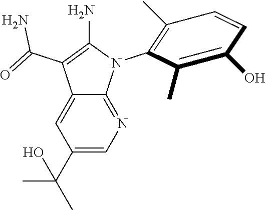 | 323 |
| 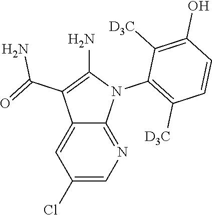 [SMILES: [2H]C([2H])([2H])c1ccc(O)c(C([2H])([2H])[2H])c1-n1c(N)c(C(N)=O)c2cc(Cl)cnc21] | 324 |
| 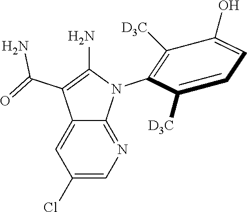 | 325 |
| 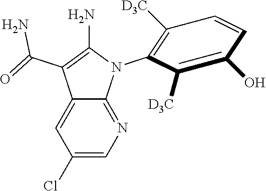 | 326 |
| 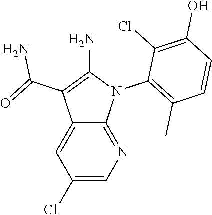 [SMILES: Cc1ccc(O)c(Cl)c1-n1c(N)c(C(N)=O)c2cc(Cl)cnc21] | 327 |
| 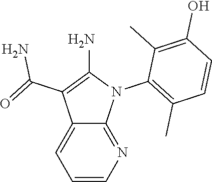 [SMILES: Cc1ccc(O)c(C)c1-n1c(N)c(C(N)=O)c2cccnc21] | 328 |

An Myt1 inhibitor may be isotopically enriched (e.g., enriched for deuterium).

Methods disclosed herein may be used for the treatment of a disease or condition which depend on the activity of membrane-associated tyrosine and threonine-specific cdc2-inhibitory kinase (Myt1) (Gene name PKMYT1) (e.g., a cancer amplified for CCNE1). Methods disclosed herein may include the step of administering to the subject in need thereof a therapeutically effective amount of a membrane-associated tyrosine and threonine-specific cdc2-inhibitory kinase (Myt1) inhibitor. Without wishing to be bound by theory, it is believed that CCNE1 amplification may result in the overexpression of CCNE1 gene products, e.g., CCNE1 transcript and/or CCNE1 protein.

Methods disclosed herein may include administration of a therapeutically effective amount of a second therapeutic. The second therapeutic may be, e.g., WEE1 inhibitor, FEN1 inhibitor, TOP1 inhibitor, RRM1 inhibitor, RRM2 inhibitor, AURKB inhibitor, TOP2A inhibitor, ATR inhibitor, TTK inhibitor, SOD1 inhibitor, SOD2 inhibitor, BUB1 inhibitor, CDC7 inhibitor, SAE1 inhibitor, PLK1 inhibitor, UBA2 inhibitor, DUT inhibitor, HDAC3 inhibitor, CHEK1 inhibitor, AURKA inhibitor, MEN1 inhibitor, DOT1L inhibitor, CREBBP inhibitor, EZH2 inhibitor, PLK4 inhibitor, HASPIN inhibitor, METTL3 inhibitor, nucleoside analog, platinum-based DNA damaging agent, or a combination thereof.

The disease or condition may have the symptom of cell hyperproliferation. For example, the disease or condition may be a cancer (e.g., a cancer amplified for CCNE1).

Cancers which have a high incidence of CCNE1 overexpression include e.g., uterine cancer, ovarian cancer, bladder cancer, pancreatic cancer, mesothelioma, kidney cancer, bladder cancer, gastric cancer, ovarian cancer, breast cancer, stomach cancer, esophageal cancer, lung cancer, and endometrial cancer. Preferably, the cancer is uterine cancer, colorectal cancer, breast cancer, lung cancer, or esophageal cancer.

Compounds disclosed herein may be administered by a route selected from the group consisting of oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, intratumoral, and topical administration.

In some embodiments, the Myt1 inhibitor is administered before the second agent (e.g., within 1 week, within 6 days, within 5 days, within 4 days, within 3 days, within 2 days, within 1 day, or within 12 hours). In some embodiments, the Myt1 inhibitor is administered after the second agent (e.g., within 1 week, within 6 days, within 5 days, within 4 days, within 3 days, within 2 days, within 1 day, or within 12 hours). In some embodiments, the Myt1 inhibitor is co-administered with the second agent. In some embodiments, the Myt1 inhibitor is administered intermittently (e.g., 1 day/week, 2 days/week, or 3 days/week). In some embodiments, the second agent is administered on a continuous daily basis.

### AURKA Inhibitors

AURKA inhibitors may be compounds that upon contacting AURKA, whether in vitro, in cell culture, or in an animal, reduce the activity of AURKA, such that the measured AURKA IC₅₀ is 10 μM or less (e.g., 5 μM or less or 1 μM or less). For certain AURKA inhibitors, AURKA IC₅₀ may be 100 nM or less (e.g., 10 nM or less, or 1 nM or less) and could be as low as 100 μM or 10 μM. Preferably, AURKA IC₅₀ is 0.1 nM to 1 M (e.g., 0.1 nM to 750 nM, 0.1 nM to 500 nM, or 0.1 nM to 250 nM). Examples of AURKA inhibitors are: MK0547, barasertib (AZD1152), PHA739358, AT9283, AMG900, SNS-314, TAK-901, CYC116, GSK1070916, PF03814735, and pharmaceutically acceptable salts thereof. Exemplary AURKA inhibitors are also disclosed in U.S. Pat. Nos. 6,977,259; 6,919,338; 7,105,669; 7,214,518; 7,235,559; 7,402,585; 7,709,479; 8,026,246; 8,138,338; 8,377,983; 9,567,329; 9,637,474; 20060178382; US20090029992; and US20190352297; the AURKA inhibitors disclosed therein are incorporated herein by reference in their entirety.

### AURKB Inhibitors

AURKB inhibitors may be compounds that upon contacting AURKB, whether in vitro, in cell culture, or in an animal, reduce the activity of AURKB, such that the measured AURKB IC₅₀ is 10 μM or less (e.g., 5 μM or less or 1 μM or less). For certain AURKB inhibitors, AURKB IC₅₀ may be 100 nM or less (e.g., 10 nM or less, or 1 nM or less) and could be as low as 100 μM or 10 μM. Preferably, AURKB IC₅₀ is 0.1 nM to 1 μM (e.g., 0.1 nM to 750 nM, 0.1 nM to 500 nM, or 0.1 nM to 250 nM). Examples of AURKB inhibitors are: MLN8237, MK0547, MLN8054, PHA739358, AT9283, AMG900, MK5108, SNS314, TAK901, CYC116, ENMD2076, and pharmaceutically acceptable salts thereof. Exemplary AURKB inhibitors are also disclosed in U.S. Pat. Nos. 7,560,551; 7,977,477; 8,110,573; and 20,110,293745; the AURKB inhibitors disclosed therein are incorporated herein by reference in their entirety.

### BUB1 Inhibitors

BUB1 inhibitors may be compounds that upon contacting BUB1, whether in vitro, in cell culture, or in an animal, reduce the activity of BUB1, such that the measured BUB1 IC₅₀ is 10 μM or less (e.g., 5 M or less or 1 μM or less). For certain BUB1 inhibitors, BUB1 IC₅₀ may be 100 nM or less (e.g., 10 nM or less, or 1 nM or less) and could be as low as 100 pM or 10 pM. Preferably, BUB1 IC₅₀ is 0.1 nM to 1 μM (e.g., 0.1 nM to 750 nM, 0.1 nM to 500 nM, or 0.1 nM to 250 nM). Examples of BUB1 inhibitors are: BAY-320, BAY-419, BAY 1816032 and pharmaceutically acceptable salts thereof. Exemplary BUB1 inhibitors are also disclosed in U.S. Pat. Nos. 9,265,763; 9,416,125; 9,745,285; 10,266,548; 10,428,044; US 20150141372; US20160145267; US20160046604; US20160046610; US20170275269; US20170305882; the BUB1 inhibitors disclosed therein are incorporated herein by reference in their entirety.

### CDC7 Inhibitors

CDC7 inhibitors may be compounds that upon contacting CDC7, whether in vitro, in cell culture, or in an animal, reduce the activity of CDC7, such that the measured CDC7 IC₅₀ is 10 μM or less (e.g., 5 μM or less or 1 μM or less). For certain CDC7 inhibitors, CDC7 IC₅₀ may be 100 nM or less (e.g., 10 nM or less, or 1 nM or less) and could be as low as 100 μM or 10 μM. Preferably, CDC7 IC₅₀ is 0.1 nM to 1 μM (e.g., 0.1 nM to 750 nM, 0.1 nM to 500 nM, or 0.1 nM to 250 nM). Examples of CDC7 inhibitors are: SRA141, TAK931, and pharmaceutically acceptable salts thereof. Exemplary CDC7 inhibitors are also disclosed in U.S. Pat. Nos. 7,279,575; 8,314,121; 8,383,624; 8,658,662; 8,691,828; 9,156,824; 9,180,105; 9,974,795; 10,745,510; US 20050043346; US20050256121; US20070293491; US20190336502; and US 20200093828; the CDC7 inhibitors disclosed therein are incorporated herein by reference in their entirety.

### CHEK1 Inhibitors

CHEK1 inhibitors may be compounds that upon contacting CHEK1, whether in vitro, in cell culture, or in an animal, reduce the activity of CHEK1, such that the measured CHEK1 IC₅₀ is 10 μM or less (e.g., 5 μM or less or 1 μM or less). For certain CHEK1 inhibitors, CHEK1 IC₅₀ may be 100 nM or less (e.g., 10 nM or less, or 1 nM or less) and could be as low as 100 μM or 10 μM. Preferably, CHEK1 IC₅₀ is 0.1 nM to 1 μM (e.g., 0.1 nM to 750 nM, 0.1 nM to 500 nM, or 0.1 nM to 250 nM). Examples of CHEK1 inhibitors are: SRA737 and pharmaceutically acceptable salts thereof. Exemplary CHEK1 inhibitors are also disclosed in U.S. Pat. Nos. 7,067,506; 8,093,244; 8,410,279; 8,530,468; 8,618,121; 8,916,591; 9,067,920; 9,440,976; 10,189,818; 10,822,327; US20090182001; US20090233896; US20090258852; US20090270416; US20090275570; US20150368244; US20180369202; and US20200397796; the CHEK1 inhibitors disclosed therein are incorporated herein by reference in their entirety.

### CREBBP Inhibitors

CREBBP inhibitors may be compounds that upon contacting CREBBP, whether in vitro, in cell culture, or in an animal, reduce the activity of CREBBP, such that the measured CREBBP IC₅₀ is 10 μM or less (e.g., 5 μM or less or 1 μM or less). For certain CREBBP inhibitors, CREBBP IC₅₀ may be 100 nM or less (e.g., 10 nM or less, or 1 nM or less) and could be as low as 100 μM or 10 μM. Preferably, CREBBP IC₅₀ is 0.1 nM to 1 μM (e.g., 0.1 nM to 750 nM, 0.1 nM to 500 nM, or 0.1 nM to 250 nM). Examples of CREBBP inhibitors are: CPI4, CCS1477, E7386, NEO1132, NEO2734, PRI724, C82, BC001, C646, EML425, CBP30, and pharmaceutically acceptable salts thereof. Exemplary CREBBP inhibitors are also disclosed in U.S. Pat. Nos. 9,763,922; 10,206,931; 10,696,655; 10,870,648; US20190270797; US20190298729; and US20190308978; the CREBBP inhibitors disclosed therein are incorporated herein by reference in their entirety.

### DOT1L Inhibitors

DOT1L inhibitors may be compounds that upon contacting DOT1L, whether in vitro, in cell culture, or in an animal, reduce the activity of DOT1L, such that the measured DOT1L IC₅₀ is 10 μM or less (e.g., 5 μM or less or 1 μM or less). For certain DOT1L inhibitors, DOT1L IC₅₀ may be 100 nM or less (e.g., 10 nM or less, or 1 nM or less) and could be as low as 100 μM or 10 μM. Preferably, DOT1L IC₅₀ is 0.1 nM to 1 μM (e.g., 0.1 nM to 750 nM, 0.1 nM to 500 nM, or 0.1 nM to 250 nM). Examples of DOT1L inhibitors are: pinometostat (EPZ5676) and pharmaceutically acceptable salts thereof. Exemplary DOT1L inhibitors are also disclosed in U.S. Pat. Nos. 8,722,877; 9,458,165; 10,112,968; US20140100184; US20150342979; and US20170335402; the DOT1L inhibitors disclosed therein are incorporated herein by reference in their entirety.

### DUT Inhibitors

DUT inhibitors may be compounds that upon contacting DUT, whether in vitro, in cell culture, or in an animal, reduce the activity of DUT, such that the measured DUT IC₅₀ is 10 μM or less (e.g., 5 μM or less or 1 μM or less). For certain DUT inhibitors, DUT IC₅₀ may be 100 nM or less (e.g., 10 nM or less, or 1 nM or less) and could be as low as 100 μM or 10 μM. Preferably, DUT IC₅₀ is 0.1 nM to 1 μM (e.g., 0.1 nM to 750 nM, 0.1 nM to 500 nM, or 0.1 nM to 250 nM). Examples of DUT inhibitors are: TAS114 and pharmaceutically acceptable salts thereof. Exemplary DUT inhibitors are also disclosed in U.S. Pat. Nos. 7,601,702; 8,530,490; 9,790,214; 9,809,571; 10,544,105; 10,562,860; 10,570,100; 10,577,321; 10,829,457; 10,858,344; US20110212467; US20190270756; US20190330158; US20190330210; and US 20200039966; the DUT inhibitors disclosed therein are incorporated herein by reference in their entirety.

### EZH2 Inhibitors

EZH2 inhibitors may be compounds that upon contacting EZH2, whether in vitro, in cell culture, or in an animal, reduce the activity of EZH2, such that the measured EZH2 IC₅₀ is 10 PM or less (e.g., 5 μM or less or 1 μM or less). For certain EZH2 inhibitors, EZH2 IC₅₀ may be 100 nM or less (e.g., 10 nM or less, or 1 nM or less) and could be as low as 100 PM or 10 μM. Preferably, EZH2 IC₅₀ is 0.1 nM to 1 μM (e.g., 0.1 nM to 750 nM, 0.1 nM to 500 nM, or 0.1 nM to 250 nM). Examples of EZH2 inhibitors are: EPZ-6438, GSK126, and pharmaceutically acceptable salts thereof. Exemplary EZH2 inhibitors are also disclosed in U.S. Pat. Nos. 8,691,507; 9,394,283; 9,889,138; 10,166,238; 10,040,782; 10,457,640; 10,633,371; 10,647,700; 10,786,511; 20,190,328743; US20190345139; and US20210052595; the EZH2 inhibitors disclosed therein are incorporated herein by reference in their entirety.

### HASPIN Inhibitors

HASPIN inhibitors may be compounds that upon contacting HASPIN, whether in vitro, in cell culture, or in an animal, reduce the activity of HASPIN, such that the measured HASPIN IC₅₀ is 10 μM or less (e.g., 5 μM or less or 1 μM or less). For certain HASPIN inhibitors, HASPIN IC₅₀ may be 100 nM or less (e.g., 10 nM or less, or 1 nM or less) and could be as low as 100 PM or 10 pM. Preferably, HASPIN IC₅₀ is 0.1 nM to 1 M (e.g., 0.1 nM to 750 nM, 0.1 nM to 500 nM, or 0.1 nM to 250 nM). Examples of HASPIN inhibitors are: SEL 120 and pharmaceutically acceptable salts thereof. Exemplary HASPIN inhibitors are also disclosed in US20130102627 and US 20130231360; the HASPIN inhibitors disclosed therein are incorporated herein by reference in their entirety.

### HDAC3 Inhibitors

HDAC3 inhibitors may be compounds that upon contacting HDAC3, whether in vitro, in cell culture, or in an animal, reduce the activity of HDAC3, such that the measured HDAC3 IC₅₀ is 10 μM or less (e.g., 5 μM or less or 1 μM or less). For certain HDAC3 inhibitors, HDAC3 IC₅₀ may be 100 nM or less (e.g., 10 nM or less, or 1 nM or less) and could be as low as 100 pM or 10 pM. Preferably, HDAC3 IC₅₀ is 0.1 nM to 1 μM (e.g., 0.1 nM to 750 nM, 0.1 nM to 500 nM, or 0.1 nM to 250 nM). Examples of HDAC3 inhibitors are: RGFP966 and pharmaceutically acceptable salts thereof. Exemplary HDAC3 inhibitors are also disclosed in U.S. Pat. Nos. 8,716,344; 9,096,549; 10,029,988; 10,059,723; and 20,190,216754; the HDAC3 inhibitors disclosed therein are incorporated herein by reference in their entirety.

### FEN1 Inhibitors

FEN1 inhibitors may be compounds that upon contacting FEN1, whether in vitro, in cell culture, or in an animal, reduce the activity of FEN1, such that the measured FEN1 IC₅₀ is 10 μM or less (e.g., 5 μM or less or 1 μM or less). For certain FEN1 inhibitors, FEN1 IC₅₀ may be 100 nM or less (e.g., 10 nM or less, or 1 nM or less) and could be as low as 100 pM or 10 pM. Preferably, FEN1 IC₅₀ is 0.1 nM to 1 μM (e.g., 0.1 nM to 750 nM, 0.1 nM to 500 nM, or 0.1 nM to 250 nM). Examples of FEN1 inhibitors are: C₈ (PMID: 32719125), SC13, FEN1-IN-3, and pharmaceutically acceptable salts thereof. Exemplary FEN1 inhibitors are also disclosed in US20200237763 and U.S. Pat. No. 7,927,790; the FEN1 inhibitors disclosed therein are incorporated herein by reference in their entirety.

### MEN1 Inhibitors

MEN1 inhibitors may be compounds that upon contacting MEN1, whether in vitro, in cell culture, or in an animal, reduce the activity of MEN1, such that the measured MEN1 IC₅₀ is 10 μM or less (e.g., 5 μM or less or 1 μM or less). For certain MEN1 inhibitors, MEN1 IC₅₀ may be 100 nM or less (e.g., 10 nM or less, or 1 nM or less) and could be as low as 100 PM or 10 pM. Preferably, MEN1 IC₅₀ is 0.1 nM to 1 μM (e.g., 0.1 nM to 750 nM, 0.1 nM to 500 nM, or 0.1 nM to 250 nM). Examples of MEN1 inhibitors are: MI3454, SNDX5613, VTP50469, KO539, and pharmaceutically acceptable salts thereof. Exemplary MEN1 inhibitors are also disclosed in U.S. Pat. Nos. 8,242,078, 9,212,180; 10,077,271; 10,526,341; 10,611,778; 10,745,409; 10,752,639; 10,781,218; 10,899,738; US20170119769; US20190010167; US 20190211036; US20200022953; US20200216471; and US20200223853; the MEN1 inhibitors disclosed therein are incorporated herein by reference in their entirety.

### METTL3 Inhibitors

METTL3 inhibitors may be compounds that upon contacting METTL3, whether in vitro, in cell culture, or in an animal, reduce the activity of METTL3, such that the measured METTL3 IC₅₀ is 10 μM or less (e.g., 5 μM or less or 1 μM or less). For certain METTL3 inhibitors, METTL3 IC₅₀ may be 100 nM or less (e.g., 10 nM or less, or 1 nM or less) and could be as low as 100 μM or 10 pM. Preferably, METTL3 IC₅₀ is 0.1 nM to 1 μM (e.g., 0.1 nM to 750 nM, 0.1 nM to 500 nM, or 0.1 nM to 250 nM). Examples of METTL3 inhibitors are: UZH1a, sTC-15, and pharmaceutically acceptable salts thereof. Exemplary METTL3 inhibitors are also disclosed in US20160264934 and WO 2020201773; the METTL3 inhibitors disclosed therein are incorporated herein by reference in their entirety.

### Nucleoside Analogs

Nucleoside analogs may be compounds that can act as antimetabolites by interfering with nucleotide production, or by acting as chain terminators in DNA lengthening by polymerase enzymes, either in cell culture, or in an animal. For certain nucleoside analogs, biological activity make occur at 10 μM or less (e.g., 5 μM or less or 1 μM or less), and could be as low as 100 pM or 10 pM. Preferably, nucleoside analog activity will occur at 1 nM to 1 μM (e.g., 1 nM to 750 nM, 1 nM to 500 nM, or 1 nM to 250 nM). Examples of nucleoside analogs are cytarabine, gemcitabine, mercaptopurine, azacytidine, cladribine, decitabine, fluorouracil, floxuridine, fludarabine or nelarabine.

### PLK1 Inhibitors

PLK1 inhibitors may be compounds that upon contacting PLK1, whether in vitro, in cell culture, or in an animal, reduce the activity of PLK1, such that the measured PLK1 IC₅₀ is 10 μM or less (e.g., 5 μM or less or 1 μM or less). For certain PLK1 inhibitors, PLK1 IC₅₀ may be 100 nM or less (e.g., 10 nM or less, or 1 nM or less) and could be as low as 100 μM or 10 μM. Preferably, PLK1 IC₅₀ is 0.1 nM to 1 μM (e.g., 0.1 nM to 750 nM, 0.1 nM to 500 nM, or 0.1 nM to 250 nM). Examples of PLK1 inhibitors are: BI2536, BI6727, TAK960, NMSP937, GSK461364, and pharmaceutically acceptable salts thereof. Exemplary PLK1 inhibitors are also disclosed in U.S. Pat. Nos. 7,504,513; 7,517,873; 7,851,495; 7,977,336; 8,101,628; 8,129,387; 8,278,299; 9,175,038; 9,175,357; US20070185133; US20080015192; US20100278833; US20150368209; US20170283445; and US20200247796; the PLK1 inhibitors disclosed therein are incorporated herein by reference in their entirety.

### PLK4 Inhibitors

PLK4 inhibitors may be compounds that upon contacting PLK4, whether in vitro, in cell culture, or in an animal, reduce the activity of PLK4, such that the measured PLK4 IC₅₀ is 10 μM or less (e.g., 5 μM or less or 1 μM or less). For certain PLK4 inhibitors, PLK4 IC₅₀ may be 100 nM or less (e.g., 10 nM or less, or 1 nM or less) and could be as low as 100 pM or 10 pM. Preferably, PLK4 IC₅₀ is 0.1 nM to 1 μM (e.g., 0.1 nM to 750 nM, 0.1 nM to 500 nM, or 0.1 nM to 250 nM). Examples of PLK4 inhibitors are: centrinone, CFI-400945, and pharmaceutically acceptable salts thereof. Exemplary PLK4 inhibitors are also disclosed in U.S. Pat. No. 10,752,612; US20190070190; and US20200383990; the PLK4 inhibitors disclosed therein are incorporated herein by reference in their entirety.

### RRM1 and RRM2 Inhibitors

RRM1 inhibitors may be compounds that upon contacting RRM1, whether in vitro, in cell culture, or in an animal, reduces the activity of RRM1, such that the measured RRM1 IC₅₀ is 10 PM or less (e.g., 5 μM or less or 1 μM or less). For certain RRM1 inhibitors, RRM1 IC₅₀ may be 100 nM or less (e.g., 10 nM or less, or 1 nM or less) and could be as low as 100 PM or 10 pM. Preferably, RRM1 IC₅₀ is 0.1 nM to 1 M (e.g., 0.1 nM to 750 nM, 0.1 nM to 500 nM, or 0.1 nM to 250 nM).

RRM2 inhibitors may be compounds that upon contacting RRM2, whether in vitro, in cell culture, or in an animal, reduce the activity of RRM2, such that the measured RRM2 IC₅₀ is 10 μM or less (e.g., 5 μM or less or 1 μM or less). For certain RRM2 inhibitors, RRM2 IC₅₀ may be 100 nM or less (e.g., 10 nM or less, or 1 nM or less) and could be as low as 100 μM or 10 μM. Preferably, RRM2 IC₅₀ is 0.1 nM to 1 μM (e.g., 0.1 nM to 750 nM, 0.1 nM to 500 nM, or 0.1 nM to 250 nM). Examples of RRM2 inhibitors are: motexafin gadolinium, hydroxyurea, fludarabine, cladribine, tezacitabine, triapine, and pharmaceutically acceptable salts thereof. Exemplary RRM2 inhibitors are also disclosed in U.S. Pat. Nos. 4,188,378; 4,357,324; and US 2019/0161461; the RRM2 inhibitors disclosed therein are incorporated herein by reference in their entirety.

### SAE1 Inhibitors

SAE1 inhibitors may be compounds that upon contacting SAE1, whether in vitro, in cell culture, or in an animal, reduce the activity of SAE1, such that the measured SAE1 IC₅₀ is 10 μM or less (e.g., 5 μM or less or 1 μM or less). For certain SAE1 inhibitors, SAE1 IC₅₀ may be 100 nM or less (e.g., 10 nM or less, or 1 nM or less) and could be as low as 100 μM or 10 μM. Preferably, SAE1 IC₅₀ is 0.1 nM to 1 μM (e.g., 0.1 nM to 750 nM, 0.1 nM to 500 nM, or 0.1 nM to 250 nM). Examples of SAE1 inhibitors are: ML792 and pharmaceutically acceptable salts thereof. Exemplary SAE1 inhibitors are also disclosed in U.S. Pat. Nos. 7,951,810; 8,008,307; 8,207,177; 9,683,003; and 9,695,154; the SAE1 inhibitors disclosed therein are incorporated herein by reference in their entirety.

### SOD1 Inhibitors

SOD1 inhibitors may be compounds that upon contacting SOD1, whether in vitro, in cell culture, or in an animal, reduce the activity of SOD1, such that the measured SOD1 IC₅₀ is 10 μM or less (e.g., 5 μM or less or 1 μM or less). For certain SOD1 inhibitors, SOD1 IC₅₀ may be 100 nM or less (e.g., 10 nM or less, or 1 nM or less) and could be as low as 100 μM or 10 μM. Preferably, SOD1 IC₅₀ is 0.1 nM to 1 μM (e.g., 0.1 nM to 750 nM, 0.1 nM to 500 nM, or 0.1 nM to 250 nM). Examples of SOD1 inhibitors are: LCS1, ATN-224, pyrimethamine, and pharmaceutically acceptable salts thereof.

SOD2 Inhibitors SOD2 inhibitors may be compounds that upon contacting SOD2, whether in vitro, in cell culture, or in an animal, reduce the activity of SOD2, such that the measured SOD2 IC₅₀ is 10 μM or less (e.g., 5 μM or less or 1 μM or less). For certain SOD2 inhibitors, SOD2 IC₅₀ may be 100 nM or less (e.g., 10 nM or less, or 1 nM or less) and could be as low as 100 μM or 10 μM. Preferably, SOD2 IC₅₀ is 0.1 nM to 1 μM (e.g., 0.1 nM to 750 nM, 0.1 nM to 500 nM, or 0.1 nM to 250 nM). Examples of SOD2 inhibitors are: LCS1, ATN-224, pyrimethamine, and pharmaceutically acceptable salts thereof.

### TOP1 Inhibitors

TOP1 inhibitors may be compounds that upon contacting TOP1, whether in vitro, in cell culture, or in an animal, reduce the activity of TOP1, such that the measured TOP1 IC₅₀ is 10 μM or less (e.g., 5 μM or less or 1 μM or less). For certain TOP1 inhibitors, TOP1 IC₅₀ may be 100 nM or less (e.g., 10 nM or less, or 1 nM or less) and could be as low as 100 μM or 10 μM. Preferably, TOP1 IC₅₀ is 0.1 nM to 1 μM (e.g., 0.1 nM to 750 nM, 0.1 nM to 500 nM, or 0.1 nM to 250 nM). Examples of TOP1 inhibitors are: irinotecan, topotecan, camptothecin, Iamellarin D, and pharmaceutically acceptable salts thereof. Exemplary TOP1 inhibitors are also disclosed in U.S. Pat. Nos. 4,604,463; 4,894,456; and 5,004,758; the TOP1 inhibitors disclosed therein are incorporated herein by reference in their entirety.

### TOP2 Inhibitors

TOP2 inhibitors may be compounds that upon contacting TOP2, whether in vitro, in cell culture, or in an animal, reduce the activity of TOP2, such that the measured TOP2 IC₅₀ is 10 μM or less (e.g., 5 μM or less or 1 μM or less). For certain TOP2 inhibitors, TOP2 IC₅₀ may be 100 nM or less (e.g., 10 nM or less, or 1 nM or less) and could be as low as 100 pM or 10 pM. Preferably, TOP2 IC₅₀ is 0.1 nM to 1 μM (e.g., 0.1 nM to 750 nM, 0.1 nM to 500 nM, or 0.1 nM to 250 nM). Examples of TOP2 inhibitors are: etoposide, teniposide, doxorubicin, daunorubicin, mitoxantrone, amsacrine, ellipticine, and pharmaceutically acceptable salts thereof. Exemplary TOP2 inhibitors are also disclosed in U.S. Pat. Nos. 3,590,028; 3,933,827; 3,989,598; 4,258,191; 4,464,529; and 4,965,348; the TOP2 inhibitors disclosed therein are incorporated herein by reference in their entirety.

### TTK Inhibitors

TTK inhibitors may be compounds that upon contacting TTK, whether in vitro, in cell culture, or in an animal, reduce the activity of TTK, such that the measured TTK IC₅₀ is 10 μM or less (e.g., 5 μM or less or 1 μM or less). For certain TTK inhibitors, TTK IC₅₀ may be 100 nM or less (e.g., 10 nM or less, or 1 nM or less) and could be as low as 100 pM or 10 pM. Preferably, TTK IC₅₀ is 0.1 nM to 1 μM (e.g., 0.1 nM to 750 nM, 0.1 nM to 500 nM, or 0.1 nM to 250 nM). Examples of TTK inhibitors are: BAY1217389 and pharmaceutically acceptable salts thereof. Exemplary TTK inhibitors are also disclosed in U.S. Pat. Nos. 8,551,980; 8,729,082; 9,199,999; 9,212,184; 9,284,317; 9,340,528; 9,388,140; 9,388,177; 9,468,642; 9,512,126; 9,512,130; 9,555,022; 9,586,958; 9,663,510; 9,670,202; US 2017/0217946; US 2017/0305912; US 2017/0334899; US 2017/0342064; U.S. Pat. No. 9,676,766; Wengner et al., Mol. Cancer Ther., 15:583-592, 2016; Zaman et al., Mol. Cancer Ther., 16:2609-2617, 2017; Mason et al., Proc. Nat'l Acad. Sci. U.S.A., 21:3127-3132, 2017; and Riggs et al., J. Med. Chem., 62:4401-4410, 2019; the TTK inhibitors disclosed therein are incorporated herein by reference in their entirety.

### UBA2 Inhibitors

UBA2 inhibitors may be compounds that upon contacting UBA2, whether in vitro, in cell culture, or in an animal, reduce the activity of UBA2, such that the measured UBA2 IC₅₀ is 10 μM or less (e.g., 5 μM or less or 1 μM or less). For certain UBA2 inhibitors, UBA2 IC₅₀ may be 100 nM or less (e.g., 10 nM or less, or 1 nM or less) and could be as low as 100 μM or 10 μM. Preferably, UBA2 IC₅₀ is 0.1 nM to 1 μM (e.g., 0.1 nM to 750 nM, 0.1 nM to 500 nM, or 0.1 nM to 250 nM). Examples of UBA2 inhibitors are: TAK981 and pharmaceutically acceptable salts thereof. Exemplary UBA2 inhibitors are also disclosed in U.S. Pat. No. 9,045,483; the UBA2 inhibitors disclosed therein are incorporated herein by reference in their entirety.

### WEE1 Inhibitors

WEE1 inhibitors may be compounds that upon contacting WEE1, whether in vitro, in cell culture, or in an animal, reduce the activity of WEE1, such that the measured WEE1 IC₅₀ is 10 μM or less (e.g., 5 μM or less or 1 μM or less). For certain WEE1 inhibitors, WEE1 IC₅₀ may be 100 nM or less (e.g., 10 nM or less, or 1 nM or less) and could be as low as 100 μM or 10 μM. Preferably, WEE1 IC₅₀ is 0.1 nM to 1 μM (e.g., 0.1 nM to 750 nM, 0.1 nM to 500 nM, or 0.1 nM to 250 nM). Examples of WEE1 inhibitors are: adavosertib (AZD1775), Debio-0123, ZN-c3 and pharmaceutically acceptable salts thereof. Exemplary WEE1 inhibitors are also disclosed in U.S. Pat. Nos. 8,791,125; 9,850,247, WO 2020210320; WO 2019028008; WO 2019173082; and WO 2020210377; the WEE1 inhibitors disclosed therein are incorporated herein by reference in their entirety.

### Platinum-Based DNA-Damaging Agent

Platinum-based DNA-damaging agents are coordination compounds of Pt(II) or Pt(IV), typically known in the art as platins. Platinum-based DNA-damaging agents include at least two coordination sites at the platinum center that are occupied by nitrogenous spectator ligand(s). The nitrogenous spectator ligands are monodentate or bidentate ligands, in which the donor atom is an sp3- or sp2-hybridized nitrogen atom within the ligand. Non-limiting examples of nitrogenous spectator ligands are ammonia, 1,2-cyclohexanediamine, a picoline, phenanthrin, or 1,6-hexanediamine. Non-limiting examples of platinum-based DNA-damaging agents include cisplatin, carboplatin, oxaliplatin, nedaplatin, triplatin tetranitrate, phenanthriplatin, picoplatin, and satraplatin.

### Isomers and Compositions Thereof

The invention includes (where possible) individual diastereomers, enantiomers, epimers, and atropisomers of the compounds disclosed herein, and mixtures of diastereomers and/or enantiomers thereof including racemic mixtures. Although the specific stereochemistries disclosed herein are preferred, other stereoisomers, including diastereomers, enantiomers, epimers, atropisomers, and mixtures of these may also have utility in treating diseases. Inactive or less active diastereoisomers and enantiomers may be useful, e.g., for scientific studies relating to the receptor and the mechanism of activation.

It is understood that certain molecules can exist in multiple tautomeric forms. This invention includes all tautomers even though only one tautomer may be indicated in the examples.

The invention also includes pharmaceutically acceptable salts of the compounds, and pharmaceutical compositions including the compounds and a pharmaceutically acceptable carrier. The compounds are especially useful, e.g., in certain kinds of cancer and for slowing the progression of cancer once it has developed in a patient.

The compounds disclosed herein may be used in pharmaceutical compositions including (a) the compound(s) or pharmaceutically acceptable salts thereof, and (b) a pharmaceutically acceptable carrier. The compounds may be used in pharmaceutical compositions that include one or more other active pharmaceutical ingredients. The compounds may also be used in pharmaceutical compositions in which the compound disclosed herein or a pharmaceutically acceptable salt thereof is the only active ingredient.

### Optical Isomers-Diastereomers-Geometric Isomers-Tautomers

Compounds disclosed herein may contain, e.g., one or more stereogenic centers and can occur as racemates, racemic mixtures, single enantiomers, individual diastereomers, and mixtures of diastereomers and/or enantiomers. The invention includes all such isomeric forms of the compounds disclosed herein. It is intended that all possible stereoisomers (e.g., enantiomers and/or diastereomers) in mixtures and as pure or partially purified compounds are included within the scope of this invention (i.e., all possible combinations of the stereogenic centers as pure compounds or in mixtures).

Some of the compounds described herein may contain bonds with hindered rotation such that two separate rotomers, or atropisomers, may be separated and found to have different biological activity which may be advantageous. It is intended that all of the possible atropisomers are included within the scope of this invention.

Some of the compounds described herein may contain olefinic double bonds, and unless specified otherwise, are meant to include both E and Z geometric isomers.

Some of the compounds described herein may exist with different points of attachment of hydrogen, referred to as tautomers. An example is a ketone and its enol form, known as keto-enol tautomers. The individual tautomers as well as mixtures thereof are encompassed by the invention.

Compounds disclosed herein having one or more asymmetric centers may be separated into diastereoisomers, enantiomers, and the like by methods well known in the art.

Alternatively, enantiomers and other compounds with chiral centers may be synthesized by stereospecific synthesis using optically pure starting materials and/or reagents of known configuration.

### Metabolites—Prodrugs

The invention includes therapeutically active metabolites, where the metabolites themselves fall within the scope of the claims. The invention also includes prodrugs, which are compounds that are converted to the claimed compounds as they are being administered to a patient or after they have been administered to a patient. The claimed chemical structures of this application in some cases may themselves be prodrugs.

### Isotopically Enriched Derivatives

The invention includes molecules which have been isotopically enriched at one or more position within the molecule. Thus, compounds enriched for deuterium fall within the scope of the claims.

### Methods of Preparing ATR Inhibitors

ATR inhibitors may be prepared using reactions and techniques known in the art. For example, certain ATR inhibitors may be prepared using techniques and methods disclosed in, e.g., International Application Nos. PCT/US2019/051539 and PCT/US2018/034729; U.S. Pat. Nos. 9,663,535, 9,549,932, 8,552,004, and 8,841,308; and U.S. Patent Application Publication No. 2019/0055240, each of which is incorporated by reference herein.

### Pharmaceutical Compositions

The compounds used in the methods described herein are preferably formulated into pharmaceutical compositions for administration to human subjects in a biologically compatible form suitable for administration in vivo. Pharmaceutical compositions typically include a compound as described herein and a pharmaceutically acceptable excipient. Certain pharmaceutical compositions may include one or more additional pharmaceutically active agents described herein.

The compounds described herein can also be used in the form of the free base, in the form of salts, zwitterions, solvates, or as prodrugs, or pharmaceutical compositions thereof. All forms are within the scope of the invention. The compounds, salts, zwitterions, solvates, prodrugs, or pharmaceutical compositions thereof, may be administered to a patient in a variety of forms depending on the selected route of administration, as will be understood by those skilled in the art. The compounds used in the methods described herein may be administered, for example, by oral, parenteral, buccal, sublingual, nasal, rectal, patch, pump, or transdermal administration, and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

For human use, a compound of the invention can be administered alone or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of a compound of the invention into preparations which can be used pharmaceutically.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, e.g., preservatives.

The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21st Ed., Gennaro, Ed., Lippincott Williams & Wilkins (2005), a well-known reference text in this field, and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include lubricating agents, e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6th Edition, Rowe et al., Eds., Pharmaceutical Press (2009).

These pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21st Ed., Gennaro, Ed., Lippincott Williams & Wilkins (2005), and Encyclopedia of Pharmaceutical Technology, eds. J. Swarbrick and J. C. Boylan, 1988-1999, Marcel Dekker, New York. Proper formulation is dependent upon the route of administration chosen. The formulation and preparation of such compositions is well-known to those skilled in the art of pharmaceutical formulation. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., 40 mesh.

### Dosages

The dosage of the compound used in the methods described herein, or pharmaceutically acceptable salts or prodrugs thereof, or pharmaceutical compositions thereof, can vary depending on many factors, e.g., the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds used in the methods described herein may be administered initially in a suitable dosage that may be adjusted as required, depending on the clinical response. In general, a suitable daily dose of a compound of the invention will be that amount of the compound that is the lowest dose effective to produce a therapeutic effect. Such an effective dose will generally depend upon the factors described above.

A compound of the invention may be administered to the patient in a single dose or in multiple doses. When multiple doses are administered, the doses may be separated from one another by, for example, 1-24 hours, 1-7 days, 1-4 weeks, or 1-12 months. The compound may be administered according to a schedule or the compound may be administered without a predetermined schedule. An active compound may be administered, for example, 1, 2, 3, 4, 5, 6, 7, 8, 9, 10, 11, or 12 times per day, every 2nd, 3rd, 4th, 5th, or 6th day, 1, 2, 3, 4, 5, 6, or 7 times per week, 1, 2, 3, 4, 5, or 6 times per month, or 1, 2, 3, 4, 5, 6, 7, 8, 9, 10, 11, or 12 times per year. It is to be understood that, for any particular subject, specific dosage regimes should be adjusted over time according to the individual need and the professional judgment of the person administering or supervising the administration of the compositions.

While the attending physician ultimately will decide the appropriate amount and dosage regimen, an effective amount of a compound of the invention may be, for example, a total daily dosage of, e.g., between 0.05 mg and 3000 mg of any of the compounds described herein. Alternatively, the dosage amount can be calculated using the body weight of the patient. Such dose ranges may include, for example, between 0.05-1000 mg (e.g., 0.25-800 mg). In some embodiments, 0.05, 0.1, 0.25, 0.5, 1, 2, 3, 4, 5, 10, 15, 20, 25, 30, 35, 40, 45, 50, 100, 150, 200, 250, 300, 350, 400, 450, 500, 550, 600, 650, 700, 750, 800, 850, 900, 950, or 1000 mg of the compound is administered.

### Formulations

A compound identified as capable of treating any of the conditions described herein, using any of the methods described herein, may be administered to patients or animals with a pharmaceutically-acceptable diluent, carrier, or excipient, in unit dosage form. The chemical compounds for use in such therapies may be produced and isolated by any standard technique known to those in the field of medicinal chemistry. Conventional pharmaceutical practice may be employed to provide suitable formulations or compositions to administer the identified compound to subjects in need thereof. Administration may begin before the patient is symptomatic.

Exemplary routes of administration of the compounds (e.g., a compound of the invention), or pharmaceutical compositions thereof, used in the present invention include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration. The compounds desirably are administered with a pharmaceutically acceptable carrier. Pharmaceutical formulations of the compounds described herein formulated for treatment of the disorders described herein are also part of the present invention. Oral administration is a preferred route of administration in the methods of the invention.

### Formulations for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution- or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

### Formulations for Parenteral Administration

The compounds described herein for use in the methods of the invention can be administered in a pharmaceutically acceptable parenteral (e.g., intravenous or intramuscular) formulation as described herein. The pharmaceutical formulation may also be administered parenterally (intravenous, intramuscular, subcutaneous or the like) in dosage forms or formulations containing conventional, non-toxic pharmaceutically acceptable carriers and adjuvants. In particular, formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. For example, to prepare such a composition, the compounds of the invention may be dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl, or n-propyl p-hydroxybenzoate. Additional information regarding parenteral formulations can be found, for example, in the United States Pharmacopeia-National Formulary (USP—NF), herein incorporated by reference.

The parenteral formulation can be any of the five general types of preparations identified by the USP—NF as suitable for parenteral administration:
(1) “Drug Injection:” a liquid preparation that is a drug substance (e.g., a compound of the (1) invention), or a solution thereof;
(2) “Drug for Injection:” the drug substance (e.g., a compound of the invention) as a dry solid that will be combined with the appropriate sterile vehicle for parenteral administration as a drug injection;
(3) “Drug Injectable Emulsion:” a liquid preparation of the drug substance (e.g., a compound of the invention) that is dissolved or dispersed in a suitable emulsion medium;
(4) “Drug Injectable Suspension:” a liquid preparation of the drug substance (e.g., a compound of the invention) suspended in a suitable liquid medium; and
(5) “Drug for Injectable Suspension:” the drug substance (e.g., a compound of the invention) as a dry solid that will be combined with the appropriate sterile vehicle for parenteral administration as a drug injectable suspension.

Exemplary formulations for parenteral administration include solutions of the compound prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 21st Ed., Gennaro, Ed., Lippincott Williams & Wilkins (2005) and in The United States Pharmacopeia: The National Formulary (USP 36 NF31), published in 2013.

Formulations for parenteral administration may, for example, contain excipients, sterile water, or saline, polyalkylene glycols, e.g., polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The parenteral formulation can be formulated for prompt release or for sustained/extended release of the compound. Exemplary formulations for parenteral release of the compound include: aqueous solutions, powders for reconstitution, cosolvent solutions, oil/water emulsions, suspensions, oil-based solutions, liposomes, microspheres, and polymeric gels.

The following examples are meant to illustrate the invention. They are not meant to limit the invention in any way.

### EXAMPLES

### Example 1. Design of SNV Panel Version 1

An SNV panel based on Anchored Multiplex PCR (AMP™) technology was designed to quantify genetic alterations in the ATM gene. Gene-specific primers were designed to amplify genomic regions of interesting utilizing AMP chemistry.

Gene-specific primers were designed for up to 30 single nucleotide variants (SNVs) that appear in 20-80% of the global population (as defined in the gnomAD database v.2.1.1) for ATM target.

Sample ploidy and chromosomal arm loss were assessed using gene-specific primers designed for 1000 randomly distributed SNVs throughout the genome.

### Example 2. Evaluation of SNV Panel Version 1 Performance Against Whole Genome Sequencing

The SNV panel designed according to the process outlined in Example 1 permits quantification of loss of heterozygosity and copy number loss for ATM. Furthermore, the design of the SNV Panel exhibits full exonic coverage, and also provides quantification of genome-wide SNVs for purity and ploidy calls. The performance of the SNV Panel was evaluated against whole genome sequencing (WGS) for 3 samples from subjects having various cancers. In particular, the three subjects were a female, age 46, with papillary serous carcinoma; a female, age 62, with pancreatic ductal adenocarcinoma; and a female, age 72, with metastatic breast carcinoma, ER-, PR—, HER2-.

For preparation of each SNV Panel sample, DNA obtained from the sample was amplified using the SNV Panel primers using VariantPlex® cycling, optimized for large panels of >3500 primer pairs:
i. PCR 1/2 cycle number 10/15
ii. Extension time: 15 min
iii. Anneal/extend temperature 60° C./65° C.
iv. Expanded testing of primer amounts in PCR
   Amplified DNA libraries were sequenced and normalized to 12 million reads per library.

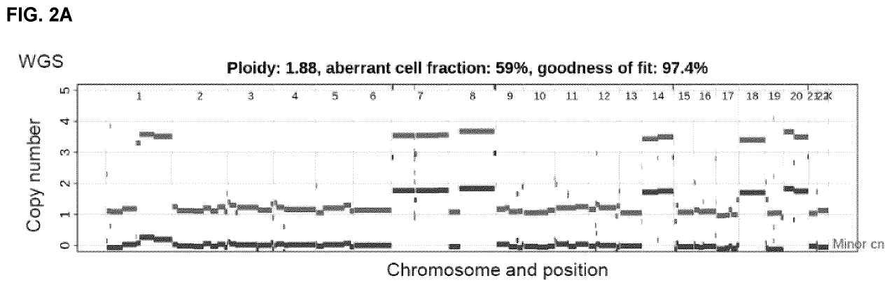

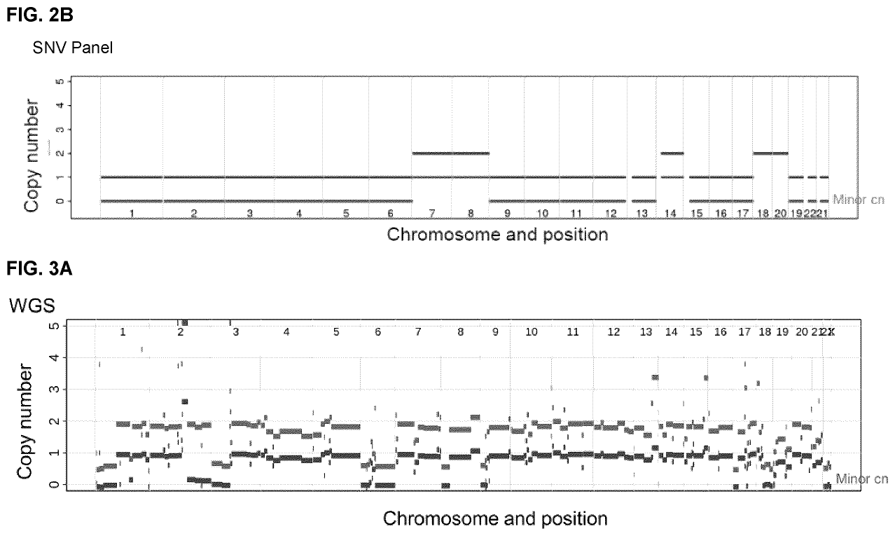

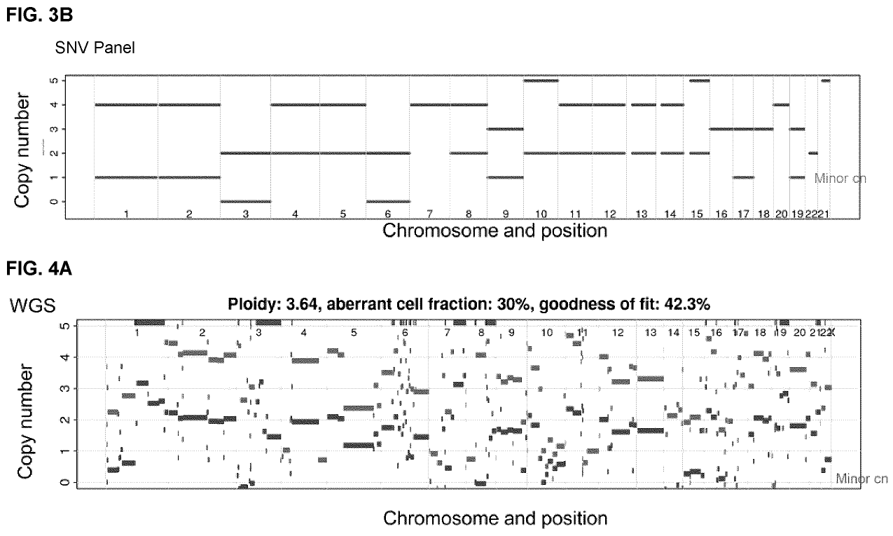

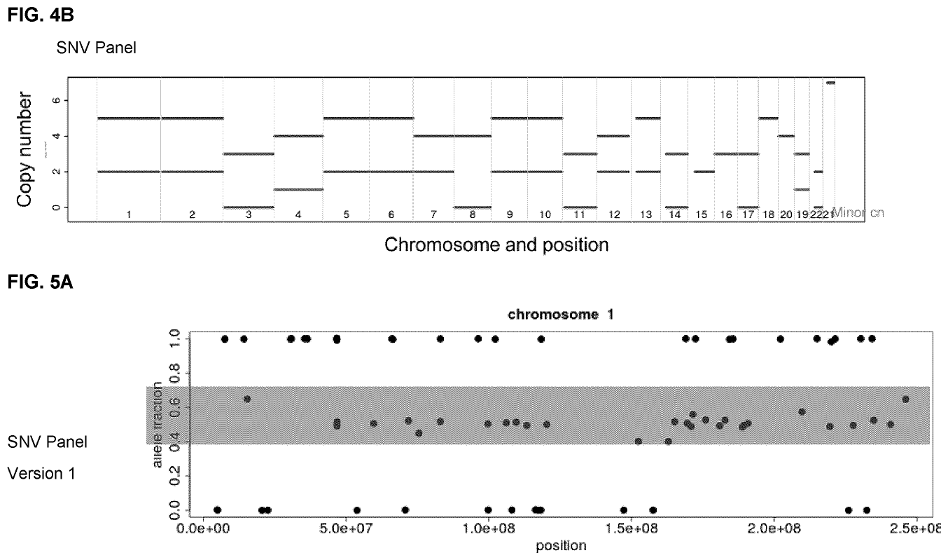

Quantification and comparison of copy number calls across all chromosomes as determined by WGS and the SNV Panel for the 3 exemplary samples are shown in FIGS. 2A, 2B, 3A, 3B, 4A, and 4B. In particular, the ability for the SNV Panel to accurately identify LOH was evaluated, with WGS serving as the basis for comparison. SNV Panel and WGS exhibited good agreement in calling LOH in the 3 exemplary samples (see Tables 3-5), particularly in tumors with simpler karyotypes and high cancer cell fractions in the sample.

TABLE 3
Concordance of LOH calls per gene
WGS

| | | LOH | Heterozygosity |
|---|---|---|---|
| SNV Panel | LOH | 35 | 0 |
| Version 1 | Heterozygosity | 0 | 5 |

TABLE 4
Concordance of LOH calls per gene
WGS

| | | LOH | Heterozygosity |
|---|---|---|---|
| SNV Panel | LOH | 8 | 1 |
| Version 1 | Heterozygosity | 4 | 27 |

TABLE 5
Concordance of LOH calls per gene
WGS

| | | LOH | Heterozygosity |
|---|---|---|---|
| SNV Panel | LOH | 17 | 5 |
| Version 1 | Heterozygosity | 1 | 17 |

### Example 3. Design of SNV Panel Version 2

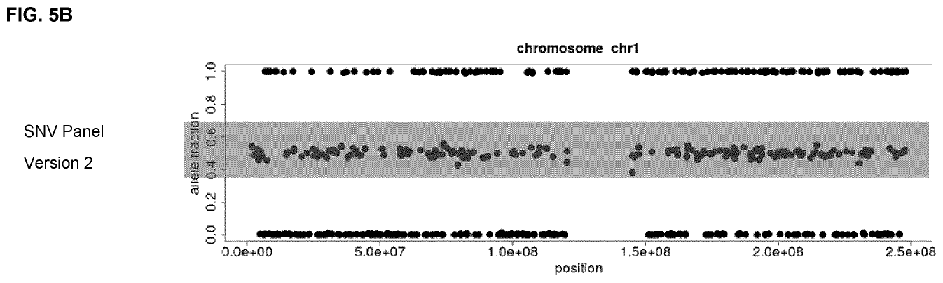

SNV Panel Version 2 was designed with increased SNV coverage. This panel has 5× increased density of heterozygous SNVs compared to SNV Panel Version 1 (See FIGS. 5A and 5B), and thus can provide more accurate identification of LOH, as evaluated against WGS.

The SNVs were selected for SNV Panel Version 2 according to the following selection criteria based on the population and sequencing characteristics.

The population characteristics were:
(1) 33%<allele frequency<66%, and
(2) 0<inbreeding coefficient<0.2.

The 5′-flanking sequence (50 base pairs) characteristics were:
(1) GC percentage between 25% and 75%,
(2) unique, and
(3) not containing other high frequency SNVs.

Additional primers were added for an additional four thousand (4000) population single nucleotide variants (SNVs) to provide additional coverage. In order to improve compatibility with low-quality FFPE input (multiple clinical sources), primer pairs in close proximity to each target are favored. In order to include SNVs which are useful for the detection of copy neutral loss of heterozygosity (LOH) structural variations, SNVs that are commonly heterozygous across sub-populations according to the current gnomAD release (v3) are selected. In order to select SNVs that are likely to yield the highest quality and quantity of NGS reads with AMP chemistry, SNVs in amplicons that are adjacent to repetitive or high GC regions of the genome were avoided, and SNVs that are less prone to noise introduced during PCR or sequencing (e.g., are not adjacent to polynucleotide tracts) are favored. Finally, in order to select SNVs that allow for spatial granularity in genomic calls, SNVs which are as evenly distributed throughout the genome as possible are selected.

Genomic DNA (>50 ng) was extracted from FFPE samples of multiple solid tumor types (n=43). Next-generation sequencing was performed on anchored multiplex PCR libraries, constructed using probes that incorporate unique molecular identifiers and span 26 genes and 5,000 genome-wide common germline SNVs. Unmatched non-tumor samples (n=24) were used to generate a reference baseline dataset. The FACETS algorithm, optimized to account for differential DNA fragmentation across samples, was used to assess copy number imbalance in heterozygous SNVs and to quantify tumor purity. Allele fractions at each heterozygous SNV were used to estimate allelic imbalances across chromosomal regions. A reference dataset was derived from matched FFPE tumor samples by whole genome sequencing (WGS) and analysis of sequence data using 3 complementary algorithms. Allele-specific copy number analysis and tumor purity estimation from SNV Panel Version 2 and WGS data were compared.

Copy number was evaluable in 605 genes from 24 matched tumor samples that passed quality control filters. Median sequencing depth across samples by SNV Panel Version 2 and WGS were 1346× and 18.6×, respectively. LOH detection by SNV Panel Version 2 was reproducible (100%) across 170 genes from 7 samples analyzed in duplicate. A strong correlation was observed between sample purity estimates by WGS and SNV Panel Version 2 (Pearson's r=0.81, p<0.001). Compared with WGS-derived calls, the sensitivity and specificity of LOH detection by SNV Panel Version 2 were 95% and 90%, respectively, rising to 97% and 91% in regions with LOH agreement by all 3 WGS algorithms, and to 99% and 97% in diploid regions with no subclonal alterations.

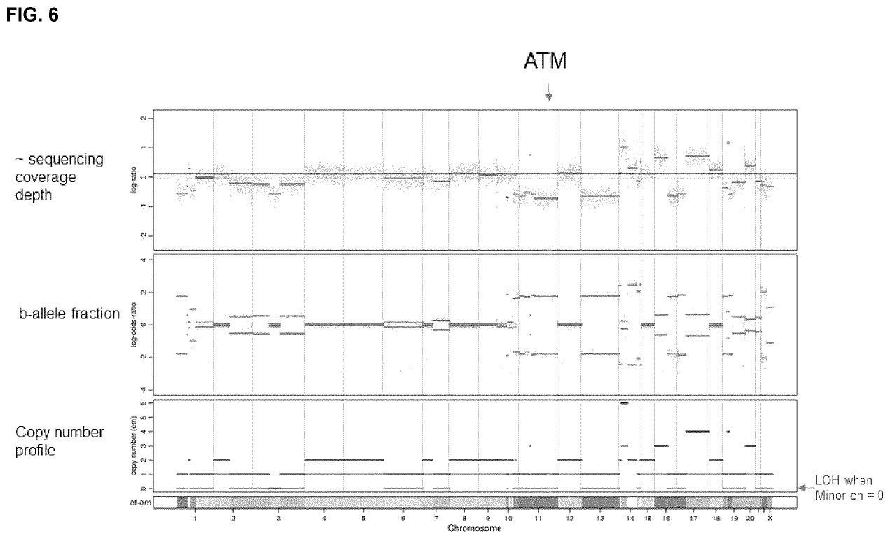

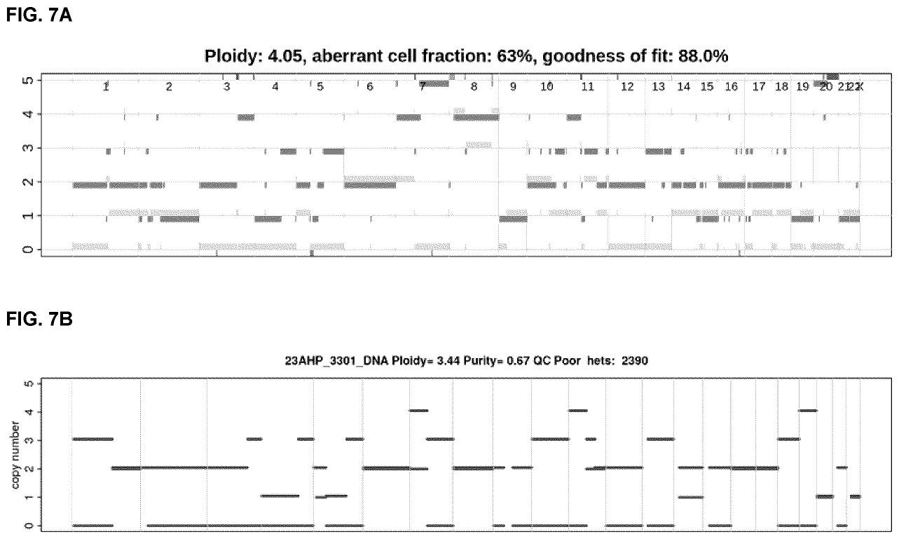

FIG. 6 demonstrates an example of copy number profile assembled using the SNV Panel Version 2 for a cancer cell having a biallelic ATM loss of function.

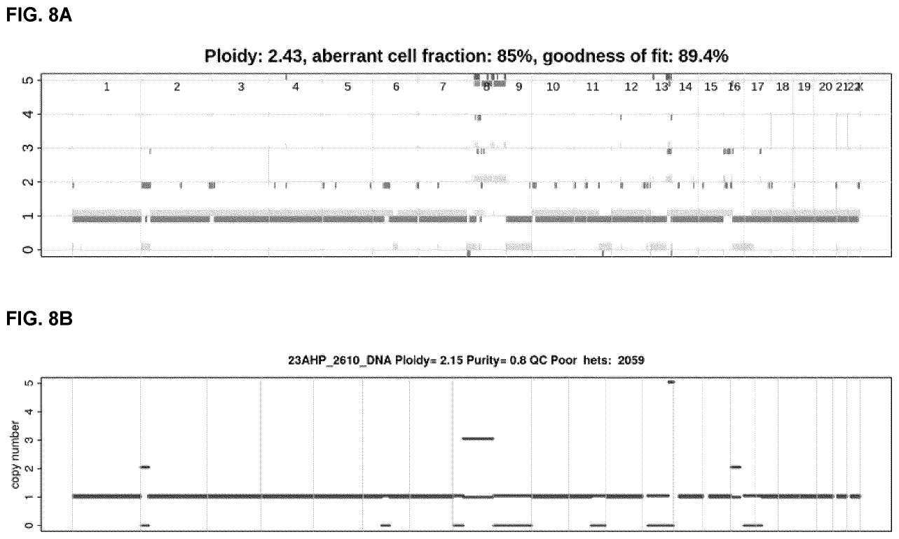

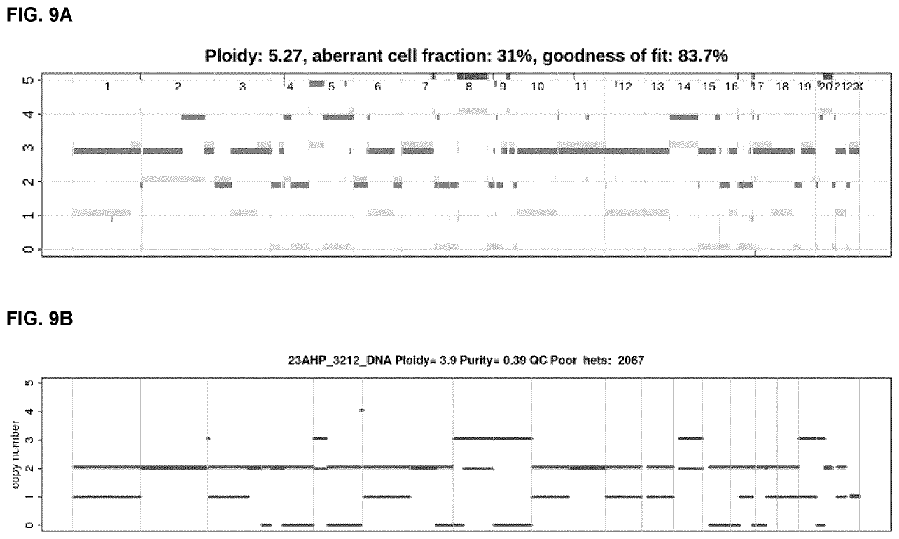

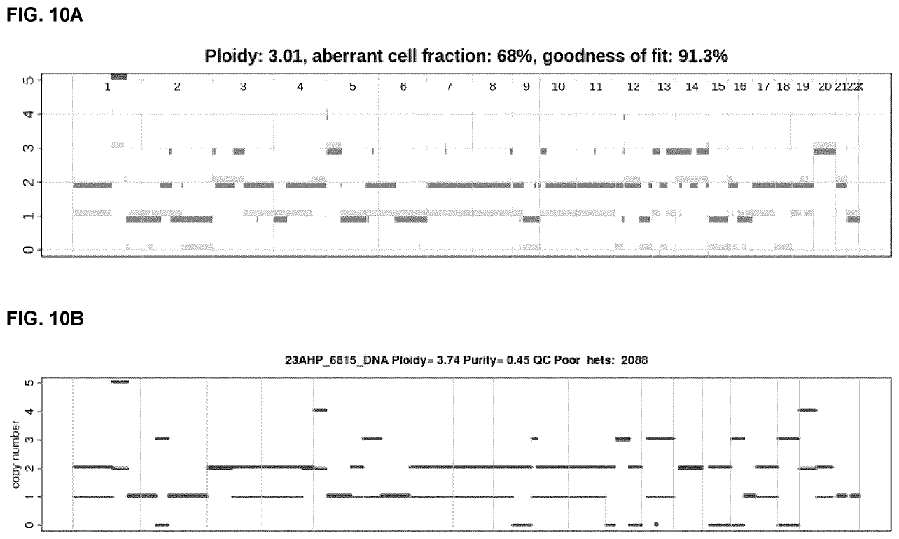

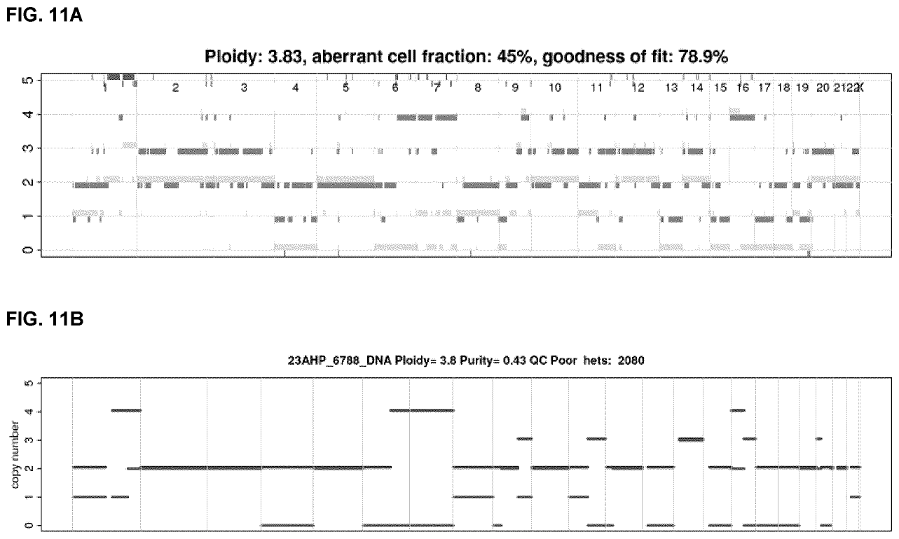

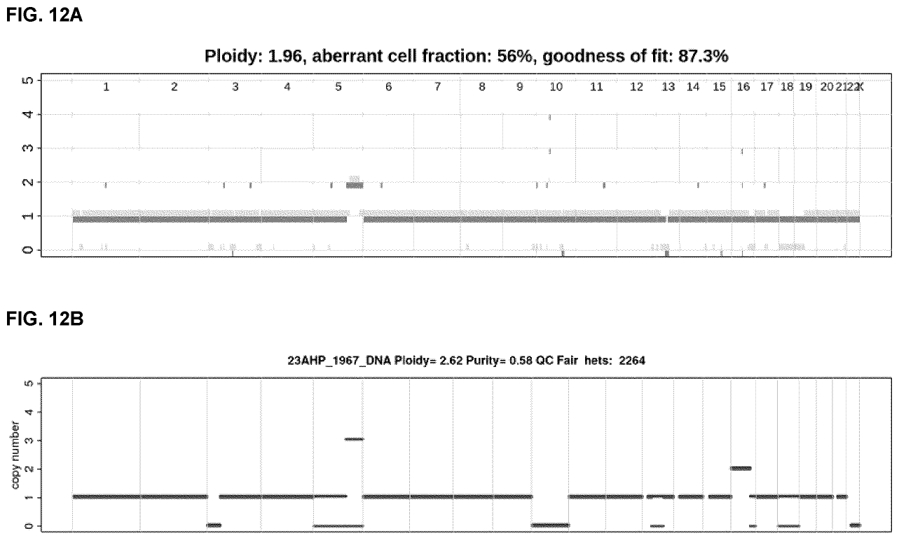

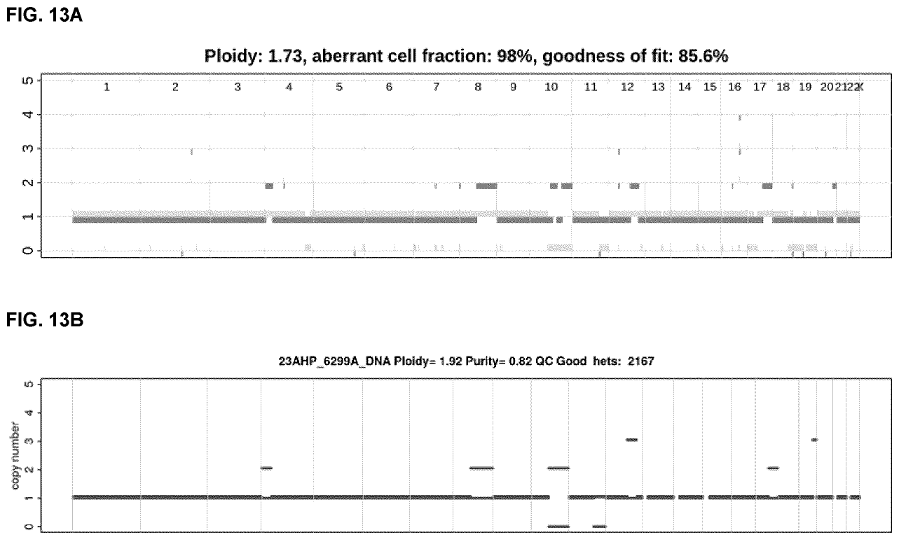

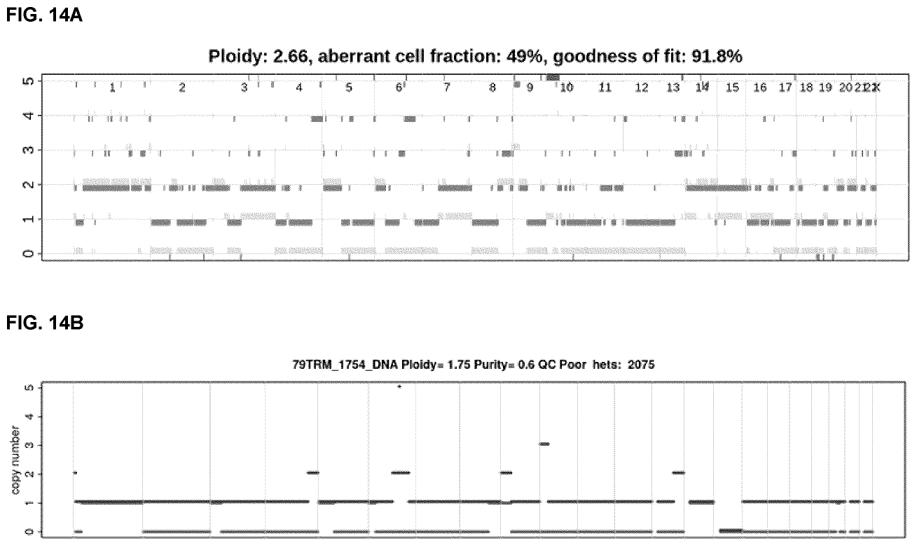

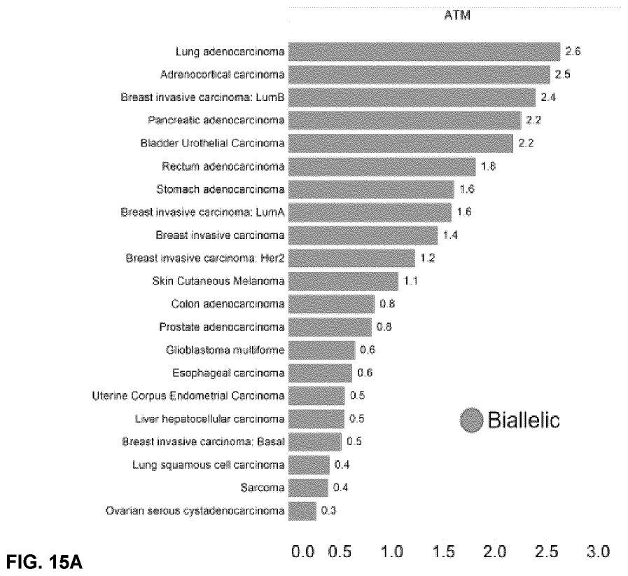

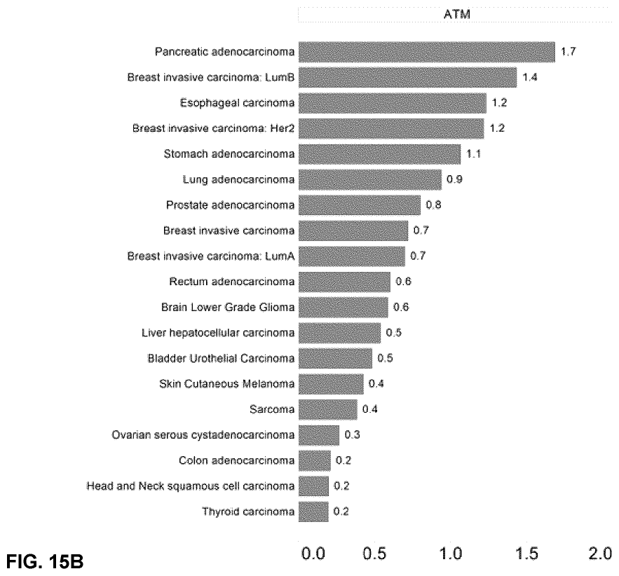

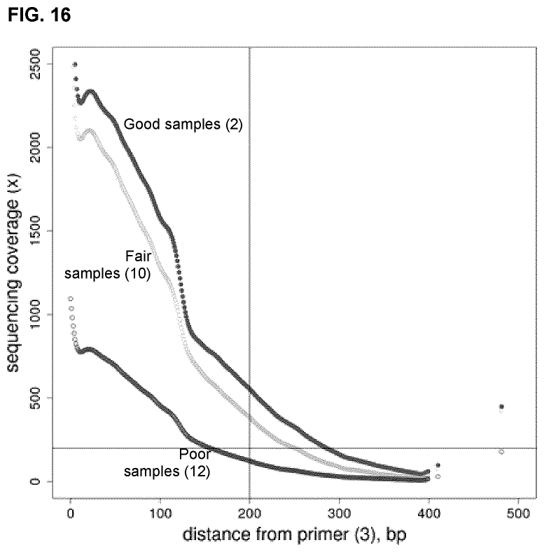

Quantification and comparison of copy number calls across all chromosomes as determined by WGS and the SNV Panel Version 2 for the 8 exemplary samples are shown in FIGS. 7A-14B. In particular, the ability for the SNV Panel Version 2 to accurately identify LOH was evaluated, with WGS serving as the basis for comparison. SNV Panel and WGS exhibited good agreement in calling LOH in the 8 exemplary samples (see Tables 6-13).

TABLE 6
Concordance of LOH calls per gene
WGS

| | | LOH | Heterozygosity |
|---|---|---|---|
| SNV Panel | LOH | 10 | 0 |
| Version 2 | Heterozygosity | 1 | 15 |

TABLE 7
Concordance of LOH calls per gene
WGS

| | | LOH | Heterozygosity |
|---|---|---|---|
| SNV Panel | LOH | 7 | 0 |
| Version 2 | Heterozygosity | 0 | 19 |

TABLE 8
Concordance of LOH calls per gene
WGS

| | | LOH | Heterozygosity |
|---|---|---|---|
| SNV Panel | LOH | 6 | 3 |
| Version 2 | Heterozygosity | 0 | 17 |

TABLE 9
Concordance of LOH calls per gene
WGS

| | | LOH | Heterozygosity |
|---|---|---|---|
| SNV Panel | LOH | 5 | 0 |
| Version 2 | Heterozygosity | 0 | 21 |

TABLE 10
Concordance of LOH calls per gene
WGS

| | | LOH | Heterozygosity |
|---|---|---|---|
| SNV Panel | LOH | 10 | 2 |
| Version 2 | Heterozygosity | 3 | 11 |

TABLE 11
Concordance of LOH calls per gene
WGS

| | | LOH | Heterozygosity |
|---|---|---|---|
| SNV Panel | LOH | 4 | 2 |
| Version 2 | Heterozygosity | 2 | 18 |

TABLE 12
Concordance of LOH calls per gene
WGS

| | | LOH | Heterozygosity |
|---|---|---|---|
| SNV Panel | LOH | 2 | 0 |
| Version 2 | Heterozygosity | 0 | 24 |

TABLE 13
Concordance of LOH calls per gene
WGS

| | | LOH | Heterozygosity |
|---|---|---|---|
| [symbol] | LOH | 19 | 4 |
| | Heterozygosity | 0 | 3 |

[symbol]

indicates data missing or illegible when filed

### Example 4. Biallelic ATM Loss of Function Mutation in Select Cancers

The frequency of biallelic ATM loss in tumors was estimated based on genomic data obtained by The Cancer Genome Atlas (Nature Genetics, 45:1113-1120, 2013). This dataset carried out molecular characterization of tumor samples from over 10,000 patients divided across 33 cancer indications. Data indicating the presence of somatic mutations, copy number variation, or loss of heterozygosity were downloaded from the Genomic Data Commons Data Portal (portal.gdc.cancer.gov). The identification of pathogenic germline mutations in TCGA samples was obtained from the published literature (Nature Commun. 8:857, 2017). Samples with co-occurrence of loss of heterozygosity and either germline or somatic pathogenic mutations were classified as biallelic loss. The mutation data was summarized across tumor indications and demonstrated that ATM biallelic loss in the TCGA cohort is more frequent in cancers such as bladder urothelial carcinoma, pancreatic adenocarcinoma, and lung adenocarcinoma. The frequencies of biallelic ATM mutations across various cancers are shown in FIGS. 7A and 7B. FIG. 7A is a chart showing the frequency of biallelic ATM mutations across cancers. FIG. 7B is a chart showing the frequency of biallelic germline ATM mutations across cancers.

### Example 5. CCNE1 Gene Overexpression Detection Using SNV Panel Version 2 and WGS

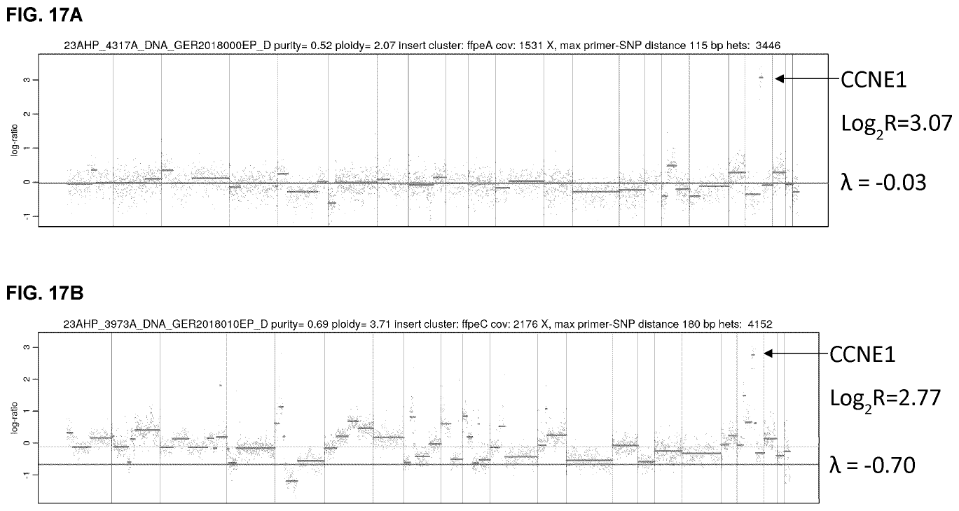

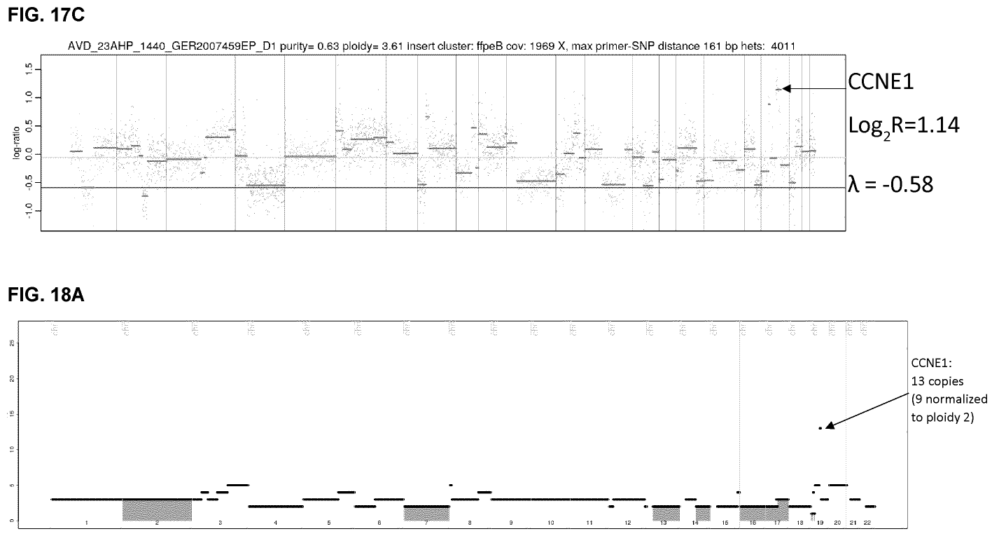

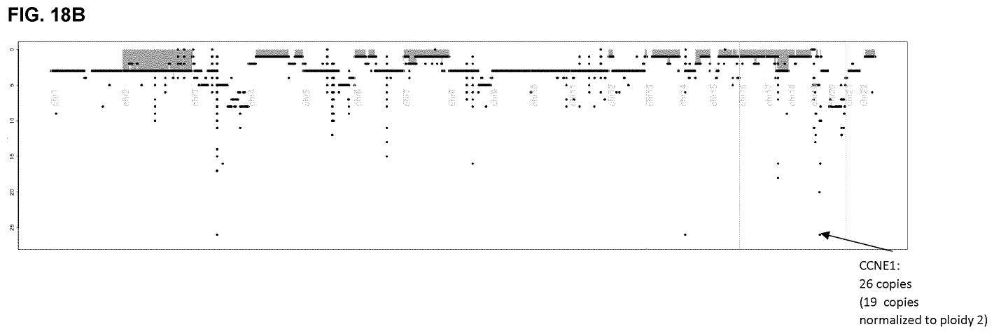

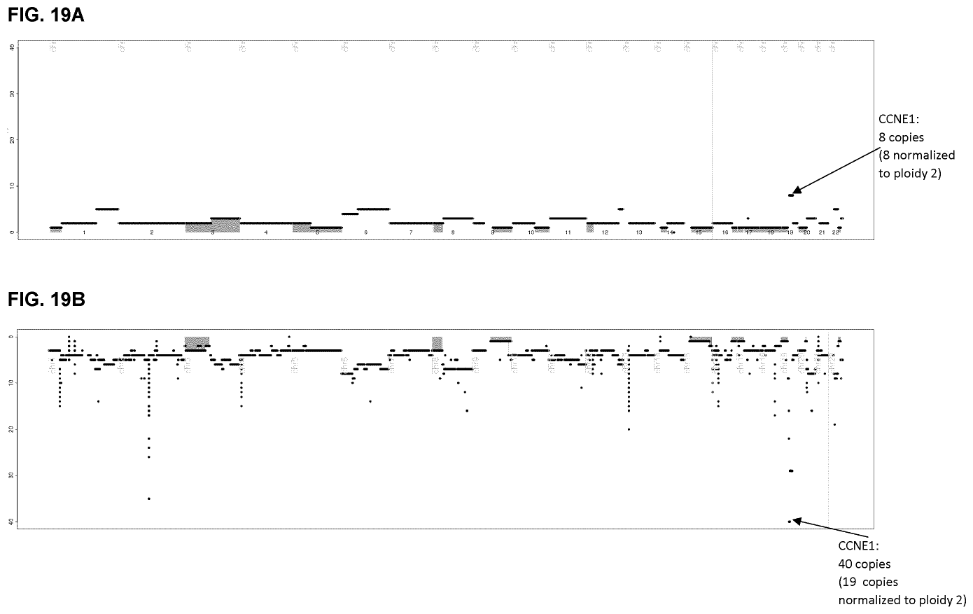

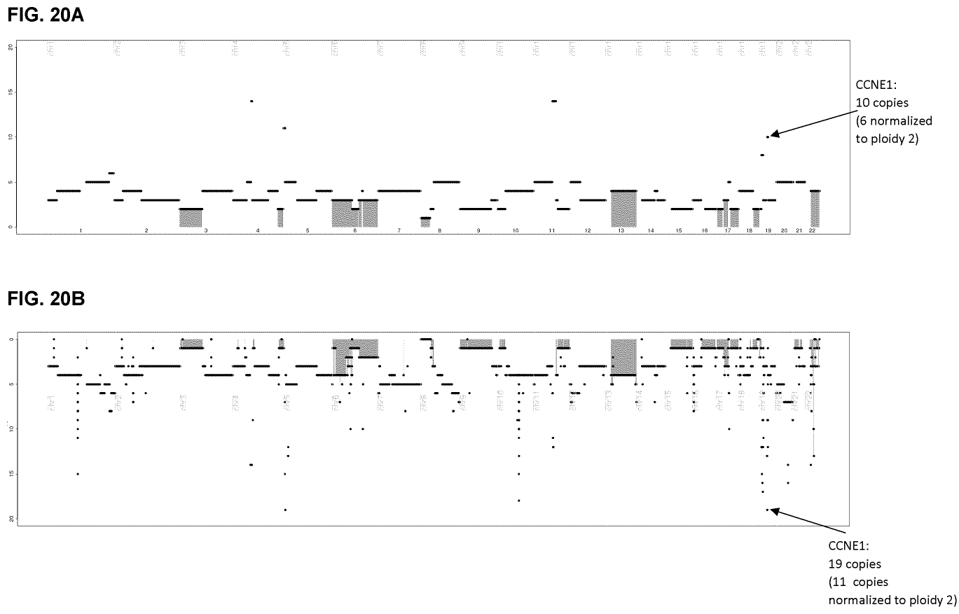

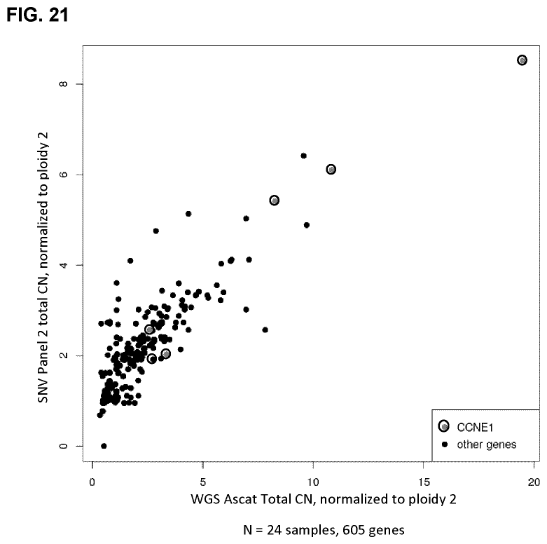

The techniques described in Example 3 were applied to detect gene overexpression. The results are shown in FIGS. 17A-21.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A method of treating a cancer having a biallelic ATM loss of function mutation in a subject, the method comprising administering to the subject in need thereof an effective amount of an ATR inhibitor.

2. A method of treating a cancer in a subject, the method comprising administering to the subject in need thereof an effective amount of an ATR inhibitor, wherein the cancer has been previously identified as having a biallelic ATM loss of function mutation.

3. A method of inducing a cell death in a cancer cell having a biallelic ATM loss of function mutation, the method comprising contacting the cell with an ATR inhibitor.

4. The method of claim 3, wherein the cell is in a subject.

5. The method of any one of claims 1 to 4, wherein the method further comprises identifying the cancer as having a biallelic ATM loss of function mutation prior to the administering step or contacting step.

6. The method of claim 5, wherein the identifying step comprises:
from read counts for a plurality of single nucleotide variants (SNVs) comprising homozygous and heterozygous SNVs obtained from sequencing a sample comprising the cancer cell and from reference read counts, determining an integer total copy number of a locus segment within an ATM gene region in a cancer cell from the subject or in the cancer cell and/or two integer allele-specific copy numbers of the locus segment,
wherein the cancer is identified as having a biallelic ATM loss of function mutation,
if at least one of the integer total copy number and the integer allele-specific copy numbers is 0, provided that the remaining ATM allele, if present, comprises an inactivating mutation, or
if none of the integer allele-specific copy numbers is 0 and ATM alleles are present, each of the ATM alleles independently having an inactivating mutation.

7. The method of claim 6, wherein the determining step comprises:
from the read counts and the reference read counts, determining total copy number log-ratios, allelic copy number log-odds ratios, and target coverage values for the SNVs;
segmenting the total copy number log-ratios and the allelic copy number log-odds ratios;
estimating sample purity and sample ploidy for the cancer cell from the total copy number log-ratios and the target coverage values; and
from the target coverage values, the sample purity, the sample ploidy, the total copy number log-ratios, and the allelic copy number log-odds ratios, generating an integer total copy number of a segment comprising a plurality of SNVs within an ATM gene region in the cancer cell and two integer allele-specific copy numbers of the segment.

8. The method of claim 7, wherein the method further comprises adjusting the ratios for location shift.

9. The method of any one of claims 6 to 8, wherein the plurality of SNVs comprises consistently covered SNVs.

10. The method of claim 9, wherein each of the consistently covered SNVs has the mean coverage of at least 200× reads across panel of normal samples.

11. The method of any one of claims 6 to 10, wherein the plurality of SNVs comprises frequent SNVs, the frequent SNVs having an allele frequency of 33% to 66% in humans.

12. The method of claim 11, wherein the plurality of SNVs comprises SNVs proximal to the frequent SNVs.

13. The method of any one of claims 6 to 11, wherein the plurality of SNVs comprises SNVs, each of the SNVs having a 5′-flanking sequence of at least 20 contiguous nucleobases comprising 25-75% GC content, wherein the 5′-flanking sequence is unique and does not comprise other SNVs.

14. The method of any one of claims 6 to 13, wherein the plurality of SNVs comprises at least 20 heterozygous SNVs.

15. The method of any one of claims 6 to 14, wherein the reference read counts are from a panel of normal samples.

16. The method of any one of claims 6 to 15, wherein the ATM gene region comprises ATM and flanking regions up to 10 kilobases each.

17. The method of any one of claims 6 to 15, wherein the ATM gene region comprises ATM and flanking regions up to 5 kilobases each.

18. The method of any one of claims 6 to 15, wherein the ATM gene region comprises ATM and flanking regions up to 2 kilobases each.

19. The method of any one of claims 6 to 18, wherein the ATM gene region is an ATM exome region.

20. The method of any one of claims 6 to 18, wherein the ATM gene region is an ATM transcriptome region.

21. The method of any one of claims 6 to 18, wherein the ATM gene region is an ATM genome region.

22. The method of any one of claims 1 to 21, wherein the biallelic ATM loss of function mutation comprises at least one somatic ATM loss of function mutation.

23. The method of any one of claims 1 to 22, wherein the biallelic ATM loss of function mutation comprises at least one germline ATM loss of function mutation.

24. The method of any one of claims 1 to 23, wherein the cancer is lung adenocarcinoma.

25. The method of any one of claims 1 to 23, wherein the cancer is adrenocortical carcinoma.

26. The method of any one of claims 1 to 23, wherein the cancer is breast invasive carcinoma.

27. The method of claim 26, wherein the cancer is LumB positive breast invasive carcinoma.

28. The method of claim 26, wherein the cancer is Her2 positive breast invasive carcinoma.

29. The method of claim 26, wherein the cancer is basal-like breast invasive carcinoma.

30. The method of any one of claims 1 to 23, wherein the cancer is pancreatic adenocarcinoma.

31. The method of any one of claims 1 to 23, wherein the cancer is bladder urothelial carcinoma.

32. The method of any one of claims 1 to 23, wherein the cancer is rectum adenocarcinoma.

33. The method of any one of claims 1 to 23, wherein the cancer is stomach adenocarcinoma.

34. The method of any one of claims 1 to 23, wherein the cancer is skin cutaneous melanoma.

35. The method of any one of claims 1 to 23, wherein the cancer is colon adenocarcinoma.

36. The method of any one of claims 1 to 23, wherein the cancer is prostate adenocarcinoma.

37. The method of any one of claims 1 to 23, wherein the cancer is glioblastoma multiforme.

38. The method of any one of claims 1 to 23, wherein the cancer is esophageal carcinoma.

39. The method of any one of claims 1 to 23, wherein the cancer is uterine corpus endometrial carcinoma.

40. The method of any one of claims 1 to 23, wherein the cancer is liver hepatocellular carcinoma.

41. The method of any one of claims 1 to 23, wherein the cancer is uterine corpus endometrial carcinoma.

42. The method of any one of claims 1 to 23, wherein the cancer is lung squamous cell carcinoma.

43. The method of any one of claims 1 to 23, wherein the cancer is a sarcoma.

44. The method of any one of claims 1 to 23, wherein the cancer is ovarian serous cystadenocarcinoma.

45. The method of any one of claims 1 to 44, wherein the ATR inhibitor is a compound of formula (I): 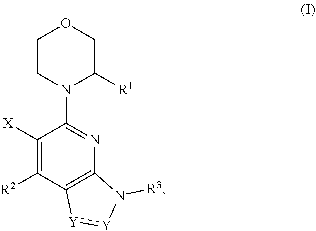
or a pharmaceutically acceptable salt thereof,
wherein [symbol]
is a double bond, and each Y is independently N or CR⁴; or [symbol] is a single bond, and each Y is independently NR^{Y}, carbonyl, or C(R^{Y})₂, wherein each R^{Y} is independently H or optionally substituted C₁₋₆ alkyl;
R¹ is optionally substituted C₁₋₆ alkyl or H;
R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
each R^{5A} is independently optionally substituted C₁₋₆ alkyl, optionally substituted C₃₋₈ cycloalkyl, or optionally substituted C₆₋₁₀ aryl;
R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
X is hydrogen or halogen.

46. The method of claim 45, wherein the ATR inhibitor is a compound of formula (II): 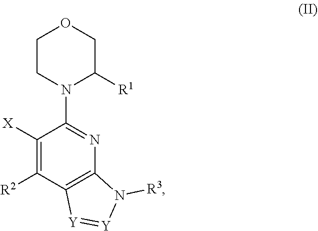
or a pharmaceutically acceptable salt thereof,
wherein
each Y is independently N or CR⁴;
R¹ is optionally substituted C₁₋₆ alkyl or H;
R² is optionally substituted C₂₋₉ heterocyclyl, optionally substituted C₁₋₆ alkyl, optionally substituted C₃₋₈ cycloalkyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁵)₂, —OR⁵, —CON(R⁶)₂, —SO₂N(R⁶)₂, —SO₂R^{5A}, or -Q-R^{5B};
R³ is optionally substituted C₁₋₉ heteroaryl or optionally substituted C₁₋₉ heteroaryl C₁₋₆ alkyl;
each R⁴ is independently hydrogen, halogen, optionally substituted C₁₋₆ alkyl, optionally substituted C₂₋₆ alkenyl, or optionally substituted C₂₋₆ alkynyl;
each R⁵ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, or —SO₂R^{5A}; or both R⁵, together with the atom to which they are attached, combine to form an optionally substituted C₂₋₉ heterocyclyl;
each R^{5A} is independently optionally substituted C₁₋₆ alkyl, optionally substituted C₃₋₈ cycloalkyl, or optionally substituted C₆₋₁₀ aryl;
R^{5B} is hydroxyl, optionally substituted C₁₋₆ alkyl, optionally substituted C₆₋₁₀ aryl, optionally substituted C₁₋₉ heteroaryl, —N(R⁵)₂, —CON(R⁶)₂, —SO₂N(R⁶)₂, —SO₂R^{5A}, or optionally substituted alkoxy;
each R⁶ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or both R⁶, together with the atom to which they are attached, combine to form an optionally substituted C₂₋₉ heterocyclyl;
Q is optionally substituted C₂₋₉ heterocyclylene, optionally substituted C₃₋₈ cycloalkylene, optionally substituted C₁₋₉ heteroarylene, or optionally substituted C₆₋₁₀ arylene; and
X is hydrogen or halogen.

47. The method of claim 45, wherein the ATR inhibitor is selected from the group consisting of compounds 43, 57, 62, 87, 93, 94, 95, 99, 100, 106, 107, 108, 109, 111, 112, 113, 114, 115, 116, 118, 119, 120, 121, 122, 123, 135, 147, 148, and pharmaceutically acceptable salts thereof from Table 1.

48. The method of claim 47, wherein the ATR inhibitor is compound 43 or a pharmaceutically acceptable salt thereof from Table 1.

49. The method of claim 47, wherein the ATR inhibitor is compound 121 or a pharmaceutically acceptable salt thereof from Table 1.

50. The method of claim 47, wherein the ATR inhibitor is compound 122 or a pharmaceutically acceptable salt thereof from Table 1.

51. The method of any one of claims 1 to 44, wherein the ATR inhibitor is 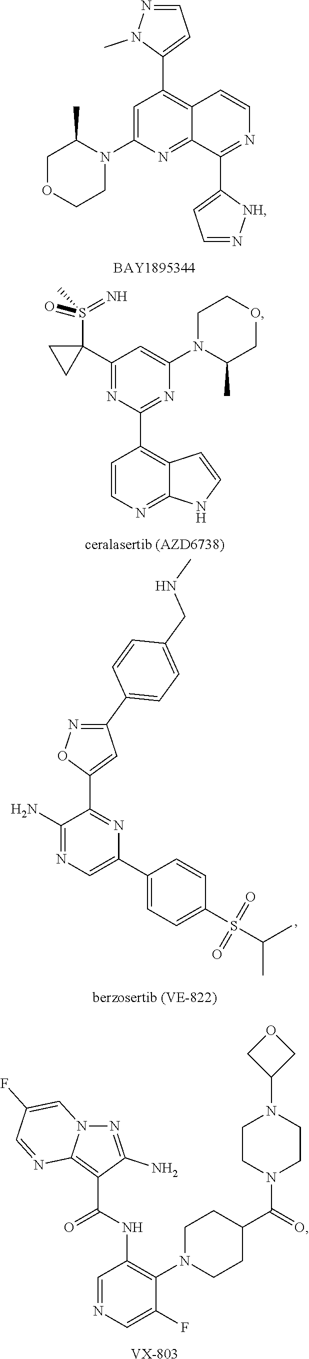
or a pharmaceutically acceptable salt thereof.

52. The method of any one of claims 1 to 51, wherein the method further comprises administering to the subject or contacting the cell with a PARP inhibitor.

53. The method of claim 52, wherein the PARP inhibitor is AZD5305, olaparib, rucaparib, veliparib (ABT-888), niraparib (ZL-2306), iniparib (BSI-201), talazoparib (BMN 673), 2X-121, CEP-9722, KU-0059436 (AZD2281), PF-01367338, or a pharmaceutically acceptable salt thereof, or a combination thereof.

54. The method of claim 52 or 53, wherein the ATR inhibitor is administered before the PARP inhibitor.

55. The method of claim 52 or 53, wherein the ATR inhibitor is administered after the PARP inhibitor.

56. The method of claim 52 or 53, wherein the ATR inhibitor is co-administered with the PARP inhibitor.

57. A method of identifying a cell from a subject as having a biallelic mutation in a target gene, the method comprising:
from read counts for a plurality of consistently covered single nucleotide variants (SNVs) comprising homozygous and heterozygous consistently covered SNVs obtained from sequencing a sample comprising the cell and from reference read counts, determining an integer total copy number of a locus segment within a target gene region in the cell from the subject and/or two integer allele-specific copy numbers of the locus segment, the target gene region comprising the mutation, wherein the reference read counts are from a panel of normal samples,
wherein the cell is identified as having a biallelic mutation for a target gene,
if at least one of the integer total copy number and the integer allele-specific copy numbers is 0, provided that the remaining target gene allele, if present, comprises the mutation, or
if none of the integer allele-specific copy numbers is 0 and target gene alleles are present, each of the target gene alleles independently having the mutation.

58. The method of claim 57, wherein the determining step comprises:
from the read counts and the reference read counts, determining total copy number log-ratios, allelic copy number log-odds ratios, and target coverage values for the SNVs;
segmenting the total copy number log-ratios and the allelic copy number log-odds ratios;
estimating sample purity and sample ploidy for the cell from the total copy number log-ratios and the target coverage values; and
from the target coverage values, the sample purity, the sample ploidy, the total copy number log-ratios, and the allelic copy number log-odds ratios, generating an integer total copy number of a segment comprising a plurality of SNVs within a target gene region in the cell and two integer allele-specific copy numbers of the segment.

59. A method of identifying a cell from a subject as amplified for a target gene, the method comprising:
from read counts for a plurality of consistently covered single nucleotide variants (SNVs) comprising homozygous and heterozygous consistently covered SNVs obtained from sequencing a sample comprising the cell and from reference read counts, determining a total copy number of a locus segment within a target gene region in the cell from the subject and a sample ploidy, the target gene region comprising the mutation, wherein the reference read counts are from a panel of normal samples,
wherein the cell is identified as amplified for a target gene,
if the total copy number is at least double the sample ploidy, or
if the total copy number is greater than the sample ploidy by at least two.

60. The method of claim 59, wherein the determining step comprises:
from the read counts and the reference read counts, determining total copy number log-ratios, and target coverage values for the SNVs;
segmenting the total copy number log-ratios;
estimating sample purity and the sample ploidy for the cell from the total copy number log-ratios and the target coverage values; and
from the target coverage values, the sample purity, the sample ploidy, and the total copy number log-ratios, generating the total copy number of a locus segment within a target gene region.

61. The method of claim 59 or 60, wherein the total copy number is a normalized total copy number.

62. The method of any one of claims 59 to 61, wherein the cell is identified as amplified for a target gene if the total copy number is at least double the sample ploidy.

63. The method of any one of claims 59 to 61, wherein the cell is identified as amplified for a target gene if the total copy number is at least triple the sample ploidy.

64. The method of claim 59 or 60, wherein the total copy number is a normalized total copy number.

65. The method of claim 59, 60, or 64, wherein the cell is identified as amplified for a target gene if the total copy number is greater than the sample ploidy by at least two.

66. The method of claim 59, 60, or 64, wherein the cell is identified as amplified for a target gene if the total copy number is greater than the sample ploidy by at least four.

67. The method of any one of claims 57 to 66, wherein the method further comprises adjusting the ratios for location shift.

68. The method of any one of claims 57 to 67, wherein the target gene is CCNE1.

69. A method of treating a cancer in a subject, the method comprising:
identifying the cancer as amplified for CCNE1 according to the method of claim 68, and
administering to the subject in need thereof a therapeutically effective amount of a membrane-associated tyrosine and threonine-specific cdc2 inhibitory kinase (Myt1) inhibitor.

70. The method of claim 69, wherein the Myt1 inhibitor is a compound of formula (III): 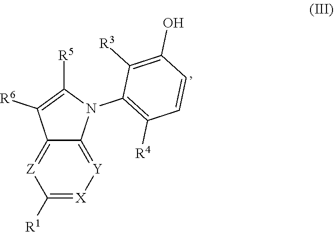
or a pharmaceutically acceptable salt thereof,
wherein
each of X, Y, and Z is independently N or CR²;
R¹ and each R² are independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, cyano, —N(R⁷)₂, —OR⁷, —C(O)N(R⁸)₂, —SO₂N(R⁸)₂, —SO₂R^{7A}, or -Q-R^{7B}; or R¹ combines with one R² that is vicinal to R¹ to form an optionally substituted C₃₋₆ alkylene;
each of R³ and R⁴ is independently optionally substituted C₁₋₆ alkyl or halogen;
R⁵ is H or —N(R⁷)₂;
R⁶ is —C(O)NH(R⁸), —C(O) R^{7A}, or —SO₂R^{7A};
each R⁷ is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or —SO₂R^{7A}; or two R⁷ groups, together with the atom to which both are attached, combine to form an optionally substituted C₂₋₉ heterocyclyl;
each R^{7A} is independently optionally substituted C₁₋₆ alkyl, optionally substituted C₃₋₈ cycloalkyl, or optionally substituted C₆₋₁₀ aryl;
each R^{7B} is independently hydroxyl, optionally substituted C₁₋₆ alkyl, optionally substituted C₆₋₁₀ aryl, optionally substituted C₂₋₉ heterocyclyl, optionally substituted C₁₋₉ heteroaryl, —N(R⁷)₂, —C(O)N(R⁸)₂, —SO₂N(R⁸)₂, —SO₂R^{7A}, or optionally substituted alkoxy;
each R⁸ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or two R⁸, together with the atom to which they are attached, combine to form an optionally substituted C₂₋₉ heterocyclyl;
Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene optionally substituted C₆₋₁₀ arylene, optionally substituted C₂₋₉ heterocyclylene, or optionally substituted C₁₋₉ heteroarylene.

71. The compound of claim 70, wherein the compound is enriched for the atropisomer of formula (IIIA): 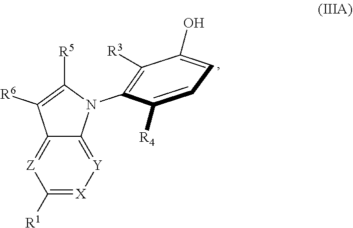
or a pharmaceutically acceptable salt thereof.

72. The method of claim 70 or 71, wherein X is CR².

73. The method of claim 70, wherein the compound is of formula (IV): 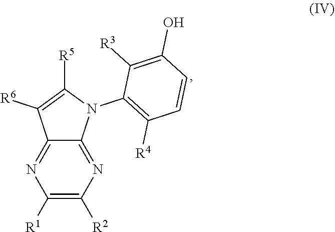
or a pharmaceutically acceptable salt thereof.

74. The method of claim 73, wherein the compound is enriched for the atropisomer of formula (IVA): 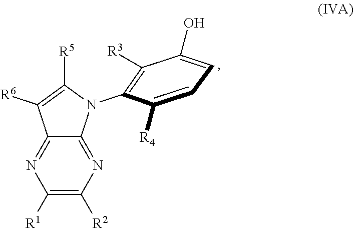
or a pharmaceutically acceptable salt thereof.

75. The method of claim 70, wherein the compound is of formula (V): 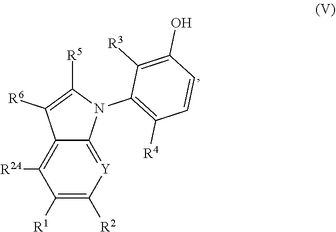
or a pharmaceutically acceptable salt thereof,
wherein R^{2A} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁷)₂, —OR⁷, —C(O)N(R⁸)₂, —SO₂N(R⁸)₂, —SO₂R^{7A}, or -Q-R^{7B}.

76. The method of claim 75, or a pharmaceutically acceptable salt thereof, wherein the compound is enriched for the atropisomer of formula (VA): 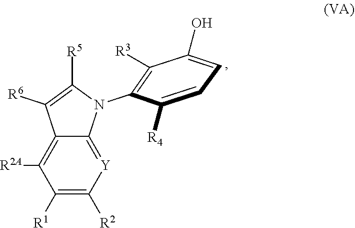
or a pharmaceutically acceptable salt thereof.

77. The method of claim 70, wherein the Myt1 inhibitor is any one of compounds 1-328 and pharmaceutically acceptable salts thereof from Table 2.

78. The method of claim 70, wherein the Myt1 inhibitor is compound 181, or a pharmaceutically acceptable salt thereof, from Table 2.

79. The method of claim 70, wherein the Myt1 inhibitor is compound 182, or a pharmaceutically acceptable salt thereof, from Table 2.

80. The method of any one of claims 57 to 79, wherein the method further comprises adjusting the ratios for location shift.

81. The method of any one of claims 57 to 80, wherein the plurality of SNVs comprises consistently covered SNVs.

82. The method of claim 81, wherein each of the consistently covered SNVs has the mean coverage of at least 200× reads across panel of normal samples.

83. The method of any one of claims 57 to 82, wherein the plurality of SNVs comprises frequent SNVs, the frequent SNVs having an allele frequency of 33% to 66% in humans.

84. The method of claim 83, wherein the plurality of SNVs comprises SNVs proximal to the frequent SNVs.

85. The method of any one of claims 57 to 84, wherein the plurality of SNVs comprises SNVs, each of the SNVs having a 5′-flanking sequence of at least 20 contiguous nucleobases comprising 25-75% GC content, wherein the 5′-flanking sequence is unique and does not comprise other SNVs.

86. The method of any one of claims 57 to 85, wherein the plurality of SNVs comprises at least 20 heterozygous SNVs.

87. The method of any one of claims 57 to 86, wherein the reference read counts are from a panel of normal samples.

88. The method of any one of claims 57 to 87, wherein the target gene region comprises the target gene and flanking regions up to 10 kilobases each.

89. The method of any one of claims 57 to 87, wherein the target gene region comprises the target gene and flanking regions up to 5 kilobases each.

90. The method of any one of claims 57 to 87, wherein the target gene region comprises the target gene and flanking regions up to 2 kilobases each.

91. The method of any one of claims 57 to 90, wherein the target gene region is a target gene exome region.

92. The method of any one of claims 57 to 90, wherein the target gene region is a target gene transcriptome region.

93. The method of any one of claims 57 to 90, wherein the target gene region is a target gene genome region.

94. A method of identifying a target mutation in a cell from a subject as being germline or somatic, the method comprising:
from read counts for a plurality of consistently covered single nucleotide variants (SNVs) comprising homozygous and heterozygous consistently covered SNVs obtained from sequencing a sample comprising the cell and from reference read counts, determining an observed allele fraction of a locus segment within a target gene region in the cell from the subject, the target gene region comprising the target mutation;
determining expected allele fractions for a germline target mutation and for a somatic target mutation;
comparing the observed allele fraction to the expected allele fractions to identify the most probable of the germline and somatic mutations; and
identifying the target mutation as germline or somatic as that which is the most probable for the germline and somatic mutations.

95. The method of claim 94, wherein the cell is in a sample from the subject, and the sample is impure (ϕ<0.9).

96. The method of claim 94 or 95, wherein the comparing step is performed using Bayesian model comparison.

97. The method of any one of claims 94 to 96, wherein each of the consistently covered SNVs has the mean coverage of at least 200× across reference non-cancerous samples.

98. The method of any one of claims 94 to 97, wherein the plurality of SNVs comprises frequent SNVs, the frequent SNVs having an allele frequency of 33% to 66% in humans.

99. The method of claim 98, wherein the plurality of SNVs comprises SNVs disposed at most 300 base pairs away from the frequent SNVs.

100. The method of any one of claims 94 to 99, wherein the plurality of SNVs comprises SNVs, each of the SNVs having a 5′-flanking sequence of at least 20 contiguous nucleobases comprising 25-75% GC content, wherein the 5′-flanking sequence is unique and does not comprise other SNVs.

101. The method of any one of claims 94 to 100, wherein the plurality of SNVs comprises at least 20 heterozygous SNVs.

102. The method of any one of claims 94 to 101, wherein the target gene region comprises the target gene and flanking regions up to 10 kilobases each.

103. The method of any one of claims 94 to 101, wherein the target gene region comprises the target gene and flanking regions up to 5 kilobases each.

104. The method of any one of claims 94 to 101, wherein the target gene region comprises the target gene and flanking regions up to 2 kilobases each.

105. The method of any one of claims 94 to 104, wherein the target gene region is a target exome region.

106. The method of any one of claims 94 to 104, wherein the target gene region is a target transcriptome region.

107. The method of any one of claims 94 to 104, wherein the target gene region is a target genome region.

108. A method of identifying a target mutation in a cell from a subject as being germline or somatic, the method comprising identifying the target mutation in the normal, matched sample from the subject,
wherein
if the target mutation present in the cell from the subject is identified in the normal, matched sample, the target mutation is germline, and
if the target mutation present in the cell from the subject is not identified in the normal, matched sample, the target mutation is somatic.

109. The method of any one of claims 57 to 108, wherein the cell from the subject is a cancer cell from the subject.

110. The method of any one of claims 1 to 109, wherein the mutation is a germline mutation.

111. The method of any one of claims 69 to 79, wherein the method further comprises administering to the subject an effective amount of a WEE1 inhibitor, FEN1 inhibitor, TOP1 inhibitor, RRM1 inhibitor, RRM2 inhibitor, AURKB inhibitor, TOP2A inhibitor, ATR inhibitor, TTK inhibitor, SOD1 inhibitor, SOD2 inhibitor, BUB1 inhibitor, CDC7 inhibitor, SAE1 inhibitor, PLK1 inhibitor, UBA2 inhibitor, DUT inhibitor, HDAC3 inhibitor, CHEK1 inhibitor, AURKA inhibitor, MEN1 inhibitor, DOT1L inhibitor, CREBBP inhibitor, EZH2 inhibitor, PLK4 inhibitor, HASPIN inhibitor, METTL3 inhibitor, nucleoside analog, platinum-based DNA damaging agent, or a combination thereof.
